(19) United States
(12) Patent Application Publication
(10) Pub. No.: US 2026/0021184 A1
(43) Pub. Date: Jan. 22, 2026

(54) Cancer Antigens and Methods

(71) Applicant: Enara Bio Limited, Oxford (GB)

(72) Inventors: Samit Kundu, Oxford (GB); Magdalena Von Essen, Oxford (GB); Rayner Myr Lauterjung Queiroz, Oxford (GB); Fabio Marino, Oxford (GB); Rose Muthoni Gathungu, Oxford (GB); Joana Senra, Oxford (GB); Masood-Ul-Hassan Zaka, Oxford (GB)

(21) Appl. No.: 19/249,526

(22) Filed: Jun. 25, 2025

Related U.S. Application Data
(63) Continuation of application No. PCT/EP2024/087738, filed on Dec. 19, 2024.

(30) Foreign Application Priority Data
Dec. 19, 2023 (EP) 23218360.8
Apr. 30, 2024 (EP) 24173560.4
Nov. 19, 2024 (EP) 24213832.9

(51) Int. Cl. A61K 40/42; A61K 39/00; A61K 40/11; A61K 40/32; C07K 14/725; C07K 16/30

(52) U.S. Cl. CPC A61K 40/428; A61K 39/001102; A61K 40/11; A61K 40/32; C07K 14/7051; C07K 16/30

(57) ABSTRACT

There are disclosed inter alia polypeptides and nucleic acids encoding said polypeptides which are useful in the treatment, prevention and diagnosis of cancer, particularly ovarian cancer, gastrointestinal cancer, pancreatic cancer, prostate cancer, stromal cancer, osteosarcoma, colon cancer, breast cancer, skin cancer, melanoma, esophageal cancer, esophageal adenocarcinoma (EAC) and esophageal squamous cell carcinoma (ESSC), particularly esophageal cancer, or EAC or ESSC.

### CROSS REFERENCE TO RELATED APPLICATIONS

The instant application is a continuation of International Application No. PCT/EP2024/087738 filed on Dec. 19, 2024, which claims the benefit of and priority to European Patent Application 24213832.9 filed on Nov. 19, 2024, European Patent Application No. 24173560.4 filed on Apr. 30, 2024, and European Patent Application No. 23218360.8 filed on Dec. 19, 2023. The entire contents of these applications are incorporated herein by reference in their entirety.

### SUBMISSION OF SEQUENCE LISTING XML

The instant application contains a Sequence Listing which has been submitted electronically in XML format and is hereby incorporated by reference in its entirety. Said XML file, created on Dec. 17, 2024, is named ENA-P3660PCT-Sequence listing and is 55, 694 bytes in size.

### FIELD OF THE INVENTION

The present invention relates to antigenic polypeptides and corresponding polynucleotides for use in the treatment or prevention of cancer, in particular for use in treating or preventing cancer, for example in ovarian cancer, gastrointestinal cancer, pancreatic cancer, prostate cancer, stromal cancer, colon cancer, breast cancer, skin cancer, melanoma, lung cancer, osteosarcoma, esophageal cancer, esophageal adenocarcinoma (EAC) and esophageal squamous cell carcinoma (ESSC), particularly esophageal cancer, or EAC or ESSC. The present invention further relates inter alia to pharmaceutical and immunogenic compositions comprising said nucleic acids and polypeptides, immune cells loaded with and/or stimulated by said polypeptides and polynucleotides, antibodies specific for said polypeptides and cells (autologous or otherwise) genetically engineered with molecules that recognize said polypeptides.

### BACKGROUND OF THE INVENTION

As part of normal immunosurveillance for pathogenic microbes, all cells degrade intracellular proteins to produce peptides that are loaded onto Major Histocompatibility Complex (MHC) Class I molecules that are expressed on the surface of all cells. Most of these peptides, which are derived from the host cell, are recognized as self, and remain invisible to the adaptive immune system. However, peptides that are foreign (non-self), are capable of stimulating the expansion of naïve CD8+ T cells that encode a T cell receptor (TCR) that tightly binds the MHC I-peptide complex. This expanded T cell population can produce effector CD8+ T cells (including Cytotoxic T-Lymphocytes-CTLs) that can eliminate the foreign antigen-tagged cells, as well as memory CD8+ T cells that can be re-amplified when the foreign antigen-tagged cells appear later in the animal's life.

MHC Class II molecules, whose expression is normally limited to professional antigen-presenting cells (APCs) such as dendritic cells (DCs), are usually loaded with peptides which have been internalised from the exogenous environment. Binding of a complementary TCR from a naïve CD4+ T cell to the MHC II-peptide complex, in the presence of various factors, including T-cell adhesion molecules (CD54, CD48) and co-stimulatory molecules (CD40, CD80, CD86), induces the maturation of CD4+ T-cells into effector cells (e.g., T_{H}1, T_{H}2, T_{H}17, T_{FH}, T_{reg} cells). These effector CD4+ T cells can promote B-cell differentiation to antibody-secreting plasma cells as well as facilitate the differentiation of antigen-specific CD8+CTLs, thereby helping induce the adaptive immune response to foreign antigens, that include both short-term effector functions and longer-term immunological memory. DCs can perform the process of cross-presentation of peptide antigens by delivering exogenously-derived antigens (such as a peptide or protein released from a pathogen or a tumor cell) onto their MHC I molecules, contributing to the generation of immunological memory by providing an alternative pathway to stimulating the expansion of naïve CD8+ T-cells.

Immunological memory (specifically antigen-specific B cells/antibodies and antigen-specific CTLs) are critical players in controlling microbial infections, and immunological memory has been exploited to develop numerous vaccines that prevent the diseases caused by important pathogenic microbes. Immunological memory is also known to play a key role in controlling tumor formation, but very few efficacious cancer vaccines have been developed.

Cancer is the second leading cause of morbidity, accounting for nearly 1 in 6 of all deaths globally. Of the 8.8 million deaths caused by cancer in 2015, the cancers which claimed the most lives were from lung (1.69 million), liver (788,000), colorectal (774,000), stomach (754,000), esophageal (439025) and breast (571,000) carcinomas. The economic impact of cancer in 2010 was estimated to be USD1.16 Trillion, and the number of new cases is expected to rise by approximately 70% over the next two decades (World Health Organisation Cancer Facts 2017).

Current therapies for cancer are varied and are highly dependent on the stage of the disease. One treatment for cancer is surgery to remove the tumor and surrounding tissue. Later stage cancer may require treatment comprising lymph node dissection, radiotherapy, or chemotherapy. Immune checkpoint blockade strategies, including the use of antibodies targeting negative immune regulators such PD-1/PD-L1 and CTLA4, have recently revolutionised treatments to a variety of malignancies (Ribas, A., & Wolchok, J. D. (2018) Science, 359:1350-1355.). The extraordinary value of checkpoint blockade therapies, and the well-recognized association of their clinical benefit with patient's adaptive immune responses (specifically T cell based immune responses) to their own cancer antigens has re-invigorated the search for effective cancer vaccines, vaccine modalities, and cancer vaccine antigens.

Historically it has been appreciated that only a small proportion of our genome is transcribed into mRNAs to produce protein and that most of the human genome consists of non-coding RNAs that are not translated into proteins. In fact around less than 1.5% of the human genome codes for proteins. The human genome is conventionally divided into the “coding” genome, which generates the approximately 20,000 annotated human protein coding genes, and the “dark” genome, which does not encode proteins. The dark genome potentially accounts for the around 98.5% of genomic space where repeat elements, enhancers, regulatory sequences, and non-coding RNAs reside. It was thought that the most important genetic regions were protein-encoding sequences, and that the remaining genome remained obscure. More recently, it has been determined that such RNA plays a critical role in diverse cellular and physiological processes including gene regulation, chromatin packaging, cell differentiation and development. Consequently, much of the human transcriptome remains hidden and uncharacterized biologically and continues to comprise the “dark matter,” of the genome. Recent advances in cancer vaccine and immunotherapy have led to the development of multiple therapeutic modalities that harness T cell immunity to recognize and eliminate cancer cells, bringing benefit to cancer patients with unmet need. However, targeted immunotherapies have utilized traditional tumor-associated antigens, which lack cancer specificity and broad expression across patient populations. It is now recognized that the genomic dark matter (genomic regions previously thought to be non-coding) offers the opportunity to identify and characterize novel, cancer-specific antigens, “Dark Antigens” uniquely presented on the surface of cancer cells and primary tumour by MHC receptors. Probing the genomic dark matter requires a complex process of creation of a pan-cancer transcriptome assembly with RNA-sequence reads from assembled cancer genome data bases for specific tumours. Transcript sequences which are subject to differential expression analysis can then be selected for those with enriched expression in tumours compared to a comprehensive panel of healthy tissues. Dark Antigens encoded by tumour-specific transcripts are then identified by translating all possible open reading frames (ORFs) and interrogating these against mass spectrometry-based immunopeptidomics data, from selected cancer samples, this permits the identification of peptides mapping to putative antigen associated ORF sequences which are then progressed to immunopeptidomic validation against transcript expression prevalence, and cancer specificity. The immunogenicity of identified Dark Antigens can be further assessed by characterization of antigen-specific T cell responses from healthy donors. Such Dark Antigens, which are cancer antigens, can form the basis of therapeutic cancer vaccines.

A wide range of vaccine modalities are known. One well-described approach involves directly delivering an antigenic polypeptide to a subject with a view to raising an immune response (including B- and T-cell responses) and stimulating immunological memory. Alternatively, a polynucleotide may be administered to the subject by means of a vector such that the polynucleotide-encoded immunogenic polypeptide is expressed in vivo. The use of viral vectors, for example adenovirus vectors, has been well explored for the delivery of antigens in both prophylactic vaccination and therapeutic treatment strategies against cancer (Wold et al. Current Gene Therapy, 2013, Adenovirus Vectors for Gene Therapy, Vaccination and Cancer Gene Therapy, 13:421-433). Immunogenic peptides, polypeptides, or polynucleotides encoding them, can also be used to load patient-derived antigen presenting cells (APCs), that can then be infused into the subject as a vaccine that elicits a therapeutic or prophylactic immune response. An example of this approach is Provenge™ (sipuleucel-T), which is presently the only FDA-approved anti-cancer vaccine.

Cancer antigens, may also be exploited in the treatment and prevention of cancer by using them to create a variety of non-vaccine therapeutic modalities. These therapies fall into two different classes: 1) antigen-binding biologics, 2) adoptive cell therapies.

Antigen-binding biologics typically comprise TCRs or antibodies or their binding domains capable of binding MHC presented antigenic peptides or epitopes (pMHC) or can consist of multivalent engineered polypeptides that recognize antigen-decorated cancer cells and facilitate their destruction. The antigen-binding components of these multivalent engineered polypeptides may consist of TCR-based biologicals, including, but not limited to TCRs, high-affinity TCRs, or pMHC binding domains thereof and TCR mimetics (e.g. antibody derived binding domains capable of recognizing and binding pMHC) produced by various technologies (including those based on monoclonal antibody technologies). Antigen-binding biologics which are multivalent engineered polypeptides may combine TCR and antibody domains in the same molecule or may combine a TCR or antibody domain with a cytolytic moiety. Cytolytic moieties of these types of multivalent biologics may consist of cytotoxic chemicals, biological toxins, targeting motifs and/or immune stimulating motifs that facilitate targeting and activation of immune cells, any of which facilitate the therapeutic destruction of tumor cells.

Adoptive cell therapies may be based on a patient's own T cells that are removed and stimulated ex vivo with vaccine antigen preparations (cultivated with T cells in the presence or absence of other factors, including cellular and acellular components) (JCI Insight. 2018 Oct. 4; 3(19). pii: 122467. doi: 10.1172/jci.insight.122467). Alternatively, adoptive cell therapies can be based on cells (including patient- or non-patient-derived cells) that have been deliberately engineered to express antigen-binding polypeptides that recognize cancer antigens. These antigen-binding polypeptides fall into the same classes as those described above for antigen-binding biologics. Thus, lymphocytes (autologous or non-autologous), that have been genetically manipulated to express cancer antigen-binding polypeptides can be administered to a patient as adoptive cell therapies to treat their cancer.

Use of HERV (human endogenous retrovirus) associated dark antigens in raising an effective immune response to cancer has shown promising results in promoting tumor regression and a more favourable prognosis in murine models of cancer (Kershaw et al., 2001, Cancer Res. 61:7920-7924; Slansky et al., 2000, Immunity 13:529-538). Further HERV associated dark antigens have been identified in WO2020/260898 (The Francis Crick Institute Ltd. and Enara Bio Ltd.) which discloses ovarian cancer antigens identified by a method involving identifying cancer-specific transcripts that entirely or partially consist of LTR elements. HERV associated dark antigen-centric immunotherapy trials have been contemplated in humans (Sacha et al., 2012, J. Immunol 189:1467-1479), although progress has been restricted, in part, due to a severe limitation of identified tumor-specific ERV antigens. It is desirable to identify further cancer-specific peptide-HLA antigens derived from aberrant epigenetic activity in previously presumed non-coding genomic regions of the tumor cell, genomic ‘dark matter’, which are distinct from the ERV associated antigens. Such novel cancer-associated antigenic sequences can be used in immunotherapy of cancer, for example ovarian cancer, gastrointestinal cancer, pancreatic cancer, prostate cancer, stromal cancer, colon cancer, breast cancer, skin cancer, melanoma, osteosarcoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

### SUMMARY OF THE INVENTION

The inventors have surprisingly discovered certain RNA transcripts which are found at high levels in ovarian cancer cells, pancreatic cancer cells, gastrointestinal cancer cells, stromal cancer cells, non small cell lung cancer (NSCLC) cells, osteosarcoma cells, colon cancer cells, breast cancer cells, skin cancer cells, melanoma cells, head and neck cancer cells, sarcoma cells, rectal cancer cells, lung cancer (e.g. LUSC, LUAD, NSCLC) cells, stomach cancer cells, cervical cancer cells, uterine cancer cells, and particularly esophageal cancer cells, such as in esophageal adenocarcinoma (EAC) and esophageal squamous cell carcinoma (ESSC), but are undetectable or found at very low levels in normal, healthy tissues (see Example 4). Further, the inventors have shown that a subset of the potential polypeptide sequences (i.e., open reading frames (ORFs)) are translated in cancer cells, processed by components of the antigen-processing apparatus, and presented on the surface of cells found in tumor tissue in association with the class I and class II major histocompatibility complex (MHC Class I, and MHC Class II) and class I and class II human leukocyte antigen (HLA Class I, HLA Class II) molecules (see Example 2). The inventors found that these peptides were mapped to ORFs which were encoded by single cancer-specific transcripts. Such transcripts are herein referred to as cancer-specific transcripts (CST). These findings demonstrate that these polypeptides (herein referred to as CST antigens) are also antigenic. Thus, cancer cell presentation of CST antigens is expected to render these cells susceptible to elimination by T cells that bear cognate T cell receptors (TCRs) for the CST antigens, and CST antigen-based vaccination methods/regimens that amplify T cells bearing these cognate TCRs are expected to elicit immune responses against cancer cells (and tumors containing them), for example ovarian cancer, gastrointestinal cancer, pancreatic cancer, prostate cancer, stromal cancer, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, and particularly esophageal cancer, esophageal adenocarcinoma (EAC) and esophageal squamous cell carcinoma (ESSC). The CSTs and the CST antigens that are the subject of the present invention are not canonical sequences which can be readily derived from known tumor genome sequences found in the cancer genome atlas. The CSTs are transcripts resulting from transcription from non-canonical ORFs. Since the CSTs are expressed at high level and since CST antigen polypeptide sequences are not sequences of normal human proteins, it is expected that they will be capable of eliciting strong, specific immune responses and thus suitable for therapeutic use in a cancer immunotherapy setting.

The CST antigens discovered in the highly expressed transcripts that characterize tumor cells, which prior to the present invention were not known to exist and produce protein products in man, can be used in several formats. First, CST antigen polypeptides of the invention can be directly delivered to a subject as a vaccine that elicits a therapeutic or prophylactic immune response to tumor cells. Second, nucleic acids of the invention, which may be codon optimised to enhance the expression of their encoded CST antigens, can be directly administered or else inserted into vectors for delivery in vivo to produce the encoded protein products in a subject as a vaccine that elicits a therapeutic or prophylactic immune response to tumor cells. Third, polynucleotides and/or polypeptides of the invention can be used to load patient-derived antigen presenting cells (APCs), that can then be infused into the subject as a vaccine that elicits a therapeutic or prophylactic immune response to tumor cells. Fourth, polynucleotides and/or polypeptides of the invention can be used for ex vivo stimulation of a subject's T cells, producing a stimulated T cell preparation that can be administered to a subject as a therapy to treat cancer. Fifth, biological molecules such as T cell receptors (TCRs) or TCR mimetics (i.e. antibody derived molecules e.g. antibody binding domains as described herein) that recognize CST antigens complexed to MHC I molecules (e.g. pMHC or peptide MHC) which have optionally been further modified to permit them to kill (or facilitate killing) of cancer cells may be administered to a subject as a therapy to treat cancer. Such biological molecules also include multivalent biologics which may comprise targeting motifs and/or immune stimulating motifs (e.g. antibody or antibody derived molecules capable of targeting/binding to immune responsive cells) that facilitate targeting and activation of immune cells combined with antigen-binding components that may consist of TCR-based biologicals, including, but not limited to TCRs, high-affinity TCRs, and TCR mimetics (for example mimetics comprising or consisting of an antibody or antibody derived molecule, e.g. a binding domain of an antibody capable of recognizing and binding to MHC presented antigen and/or peptide, e.g. pMHC) produced by various technologies, any of which may facilitate the therapeutic destruction of tumor cells. Sixth, chimeric versions of biological molecules, or biological molecules that comprise a heterologous sequence, that recognize CST antigens complexed to MHC cells may be introduced into T cells (autologous or non-autologous), and the resulting cells may be administered to a subject as a therapy to treat cancer. These and other applications are described in greater detail below.

Thus, the invention provides inter alia an isolated polypeptide comprising a sequence selected from:
(a) the sequence of any one of SEQ ID NOs. 1 to 17 and 57, and
(b) a variant of the sequence of (a); and
(c) a fragment, such as an immunogenic fragment, of the sequences of (a) and (b)
(hereinafter referred to as “a polypeptide of the invention”).

The invention also provides a nucleic acid molecule which encodes a polypeptide of the invention (hereinafter referred to as “a nucleic acid of the invention”).

The polypeptides of the invention and the nucleic acids of the invention, as well as related aspects of the invention, are expected to be useful in a range of embodiments in cancer immunotherapy and prophylaxis, particularly immunotherapy and prophylaxis of cancer especially any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

Description of the Sequences

MKRYNRIMHDELT, SEQ ID NO: 1, polypeptide sequence of CST Antigen 1
(EVA001).
VVLPGTLCFMKLLKLNMGSLAPRSALTLETPAKYVR, SEQ ID NO: 2, polypeptide
sequence of CST Antigen 2 (EVA002).
LAANSVSVTSVPLWCLYSISLASSLMHPKTDAALLSPVPSQQTRGCFYPCLDF,
SEQ ID NO: 3, polypeptide sequence of CST Antigen 3 (EVA003).
LARETLLPASSAFPSSPFRTVPHLLLAQCCLKMQLLFANNRNTS, SEQ ID NO: 4,
polypeptide sequence of CST Antigen 4 (EVA004).
LSGMSLSGVMIKLYSPLFMTRYNSPSPLKSQLLVSLSWTSVIGRLLMSIFNLC, SEQ
ID NO: 5, polypeptide sequence of CST Antigen 5 (EVA005).
MLKTNNKKQTTKKKLKKKTEIEL, SEQ ID NO: 6, polypeptide sequence of CST
Antigen 6 (EVA006).
MLIAVSLTILAVLIFWIN, SEQ ID NO: 7, polypeptide sequence of CST Antigen 7
(EVA007).
MQKVPDVLWNRKLRKKKRSFKRSSPVNSVLSKHWTRSASR, SEQ ID NO: 8,
polypeptide sequence of CST Antigen 8 (EVA008).
VEMPAIKWHPDGLIHITLL, SEQ ID NO: 9, polypeptide sequence of CST Antigen 9
(EVA009).
MSQGRASPFTTSRPRPGKGSLWRRRFVREEGEAPTHLGLTRSFAKRLGGVPGAT
KLPPS, SEQ ID NO: 10, polypeptide sequence of CST Antigen 10 (EVA010).
MDSPIPYHLSPTLAPEILNSTM, SEQ ID NO: 11, polypeptide sequence of CST
Antigen 11 (EVA011).
TFLELKLFSTEEDRADSRDHGVSLGPSPQMVHPLAEAPAHSLTSNLLEPAHHCQAH
Y, SEQ ID NO: 12, polypeptide sequence of CST Antigen 12 (EVA012).
VQNSVDSSLSLLWRSSIPYLEMTLFMCLSLVMML, SEQ ID NO: 13, polypeptide
sequence of CST Antigen 13 (EVA013).
MASRKKMIGFVK, SEQ ID NO: 14, polypeptide sequence of CST Antigen 14
(EVA014).
LFKMPQVKHQRRNLRDPLPNRSLIKQRPPRKWQSPTLASFQLGSRLLTTPPCPTRK,
SEQ ID NO: 15, polypeptide sequence of CST Antigen 15 (EVA015).
MPGRPPPTVGPLISPVAPRLPFTFGPSKLKSTFGAEIRGPFRFTSTPGPFSSPETCG
PLALTCKSDPGVEMPNWGSFILGSLMLPSDASKLRSGAPTDTLGHLMSPETARSP
CRLGPLSVTSGAPTLSFVVASIPGTFTPSPPTCILPPTPLRLSPGACTFMLGTDASR
SPFKLGPFKFRSRSGPETFGIERFTEGKSTPGPFRETSGPSSLTSGPLKSPSREGS
WAWTSAPPPSIFTPGMPILGME, SEQ ID NO: 16, polypeptide sequence of CST
Antigen 16 (EVA016).
VYNIKMKGFVLHCEVQAEVGIRF, SEQ ID NO: 17, polypeptide sequence of CST
Antigen 17 (EVA017).
KRYNRIMHDEL, SEQ ID NO: 18, peptide sequence derived from CST Antigen 1
(EVA001).
KLLKLNMGSL, SEQ ID NO: 19, peptide sequence derived from CST Antigen 2
(EVA002).
HPKTDAALL, SEQ ID NO: 20, peptide sequence derived from CST Antigen 3
(EVA003).
SSLMHPKTDAAL, SEQ ID NO: 21, peptide sequence derived from CST Antigen 3
(EVA003).
FPSSPFRTV, SEQ ID NO: 22, peptide sequence derived from CST Antigen 4
(EVA004).
FRTVPHLLL, SEQ ID NO: 23, peptide sequence derived from CST Antigen 4
(EVA004).
SPSPLKSQL, SEQ ID NO: 24, peptide sequence derived from CST Antigen 5
(EVA005).
LKKKTEIEL, SEQ ID NO: 25, peptide sequence derived from CST Antigen 6
(EVA006).
AVSLTILAV, SEQ ID NO: 26, peptide sequence derived from CST Antigen 7
(EVA007).
KRSSPVNSV, SEQ ID NO: 27, peptide sequence derived from CST Antigen 8
(EVA008).
KRSSPVNSVLSK, SEQ ID NO: 28, peptide sequence derived from CST Antigen 8
(EVA008).
GLIHITLL, SEQ ID NO: 29, peptide sequence derived from CST Antigen 9
(EVA009).
HPDGLIHITL, SEQ ID NO: 30, peptide sequence derived from CST Antigen 9
(EVA009).
RPRPGKGSL, SEQ ID NO: 31, peptide sequence derived from CST Antigen 10
(EVA010).
SPTLAPEIL, SEQ ID NO: 32, peptide sequence derived from CST Antigen 11
(EVA011).
SRDHGVSL, SEQ ID NO: 33, peptide sequence derived from CST Antigen 12
(EVA012).
VSLGPSPQMV, SEQ ID NO: 34, peptide sequence derived from CST Antigen 12
(EVA012).
VDSSLSLL, SEQ ID NO: 35, peptide sequence derived from CST Antigen 13
(EVA013).
MASRKKMI, SEQ ID NO: 36, peptide sequence derived from CST Antigen 14
(EVA014).
SLIKQRPPRK, SEQ ID NO: 37, peptide sequence derived from CST Antigen 15
(EVA015).
RSLIKQRPPR, SEQ ID NO: 38, peptide sequence derived from CST Antigen 15
(EVA015).
SLIKQRPPR, SEQ ID NO: 39, peptide sequence derived from CST Antigen 15
(EVA015).
DPLPNRSLI, SEQ ID NO: 40, peptide sequence derived from CST Antigen 15
(EVA015).
RSLIKQRPPRK, SEQ ID NO: 41, peptide sequence derived from CST Antigen 15
(EVA015).
ILPPTPLRL, SEQ ID NO: 42, peptide sequence derived from CST Antigen 16
(EVA016).
GAPTLSFVVA, SEQ ID NO: 43, peptide sequence derived from CST Antigen 16
(EVA016).
GSFILGSLM, SEQ ID NO: 44, peptide sequence derived from CST Antigen 16
(EVA016).
LMLPSDASKLR, SEQ ID NO: 45, peptide sequence derived from CST Antigen 16
(EVA016).
LRSGAPTDTL, SEQ ID NO: 46, peptide sequence derived from CST Antigen 16
(EVA016).
RFTEGKSTPGPFR, SEQ ID NO: 47, peptide sequence derived from CST Antigen
16 (EVA016).
ILPPTPLRL, SEQ ID NO: 48, peptide sequence derived from CST Antigen 16
(EVA016).
KLKSTFGAEI, SEQ ID NO: 49, peptide sequence derived from CST Antigen 16
(EVA016).
SFILGSLML, SEQ ID NO: 50, peptide sequence derived from CST Antigen 16
(EVA016).
GAPTLSFVVA, SEQ ID NO: 51, peptide sequence derived from CST Antigen 16
(EVA016).
GSFILGSLM, SEQ ID NO: 52, peptide sequence derived from CST Antigen 16
(EVA016).
TSGPLKSPSR, SEQ ID NO: 53, peptide sequence derived from CST Antigen 16
(EVA016).
VQAEVGIRF, SEQ ID NO: 54, peptide sequence derived from CST Antigen 17
(EVA017).
DPLPNRSLI, SEQ ID NO: 55, peptide sequence derived from CST Antigen 15
(EVA015).
KRSSPVNSVLSK, SEQ ID NO: 56, peptide sequence derived from CST Antigen 8
(EVA008).
MDPPGHQGLQVQEATLESLVFQGPLGLQAHQVKQSCLRVL, SEQ ID NO: 57,
polypeptide sequence of CST Antigen 18 (EVA018).
GPLGLQAHQV, SEQ ID NO: 58, peptide sequence derived from CST Antigen 18
(EVA018).
AKFVAAWTLKAAA, SEQ ID NO: 59, PADRE epitope sequence,
PKYVKQNTLKLAT, SEQ ID NO: 60, HA307-319 epitope sequence,
QYIKANSKFIGITE, SEQ ID NO: 61 TT830-843 epitope sequence,
FNNFTVSFWLRVPKVSASHLE, SEQ ID NO: 62, VP2 epitope sequence,
ISQAVHAAHAEINEAGR, SEQ ID NO: 63, ovalbumin epitope sequence,

### DESCRIPTION OF THE FIGURES

### List of Abbreviations of Cancer Types and Cells Used in Figures

ACC—Adrenocortical carcinoma
BLCA—Bladder Urothelial Carcinoma
BRCA—Breast invasive carcinom
CESC—Cervical squamous cell carcinoma and endocervical adenocarcinoma
CHOL—Cholangiocarcinoma (bile duct)
COAD—Colon adenocarcinom
DLBC—Lymphoid neoplasm diffuse large B-cell lymphoma
ESCA—Esophageal carcinoma
GBM—Glioblastoma multiforme
H&N—Head and Neck
HNSC—Head and Neck squamous cell carcinoma LAML-Acute Myeloid Leukemia
KICH—Kidney Chromophobe
KIRC—Kidney renal clear cell carcinoma
KIRP—Kidney renal papillary cell carcinoma
LAML—Acute Myeloid Leukemia
LGG—Low-grade glioma
LIHC—Liver hepatocellular carcinoma
LCA—Should it be BLCA-Bladder Urothelial Carcinoma
LUAD—Lung adenocarcinoma
LUSC—Lung squamous cell carcinoma
MESO—Mesothelioma
OV—Ovarian serous cystadenocarcinoma
PAAD—Pancreatic adenocarcinoma
PCPG—Pheochromocytoma and Paraganglioma
PRAD—Prostate adenocarcinoma
READ—Rectum adenocarcinoma
SARC—Sarcoma
SKCM—Skin Cutaneous Melanoma
STAD—Stomach adenocarcinoma
TGCT—Testicular Germ Cell Tumors
THCA—Thyroid carcinoma
THYM—Thymoma
UCS/UCSC—Uterine Carcinosarcoma
UVM—Uveal Melanoma
SkMus=Skin Muscle
Liv=Liver
Pro=Prostate
Lun=Lung
Spl=Spleen
Kid=Kidney
Bra=Brain
Hea=Head and Neck
Flo-1=human esophageal adenocarcinoma
OE19=human esophageal adenocarcinoma
OE33=human esophageal adenocarcinoma
OACM5.1C=human esophageal adenocarcinoma
SK-GT4=human distal oesophageal carcinoma
ESO51=human esophageal adenocarcinoma
ESO62=Adenocarcinoma of the gastroesophageal junction
KYAE-1=oesophageal adenocarcinoma
EBC-1=lung squamous cell carcinoma cell line
SKL-U1=adenocarcinoma
HCT116=colon carcinoma
DMS53=colon carcinoma
CL-40=
RPMI7951=melanoma
HARA=Epstein-Barr virus (EBV) transformed lymphoblastoid cell line

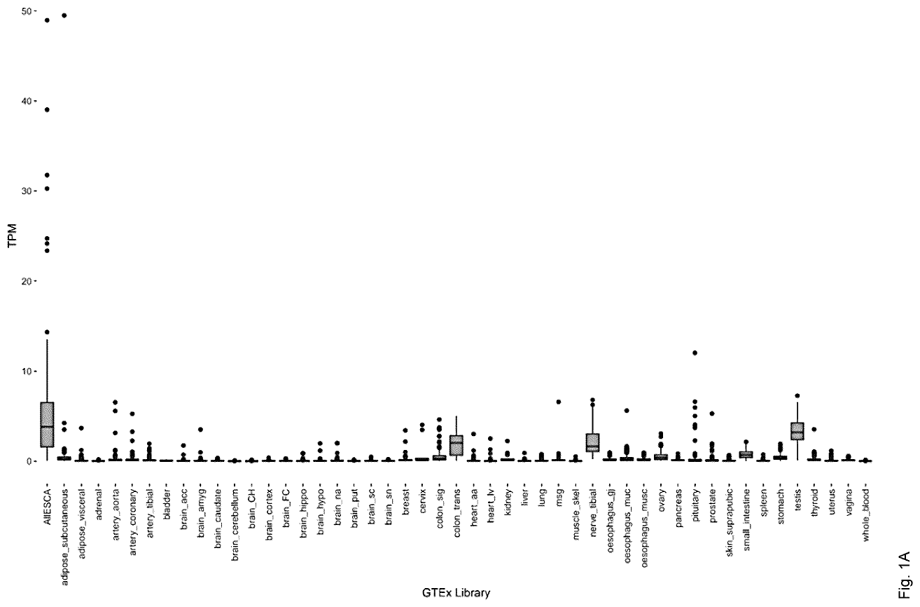

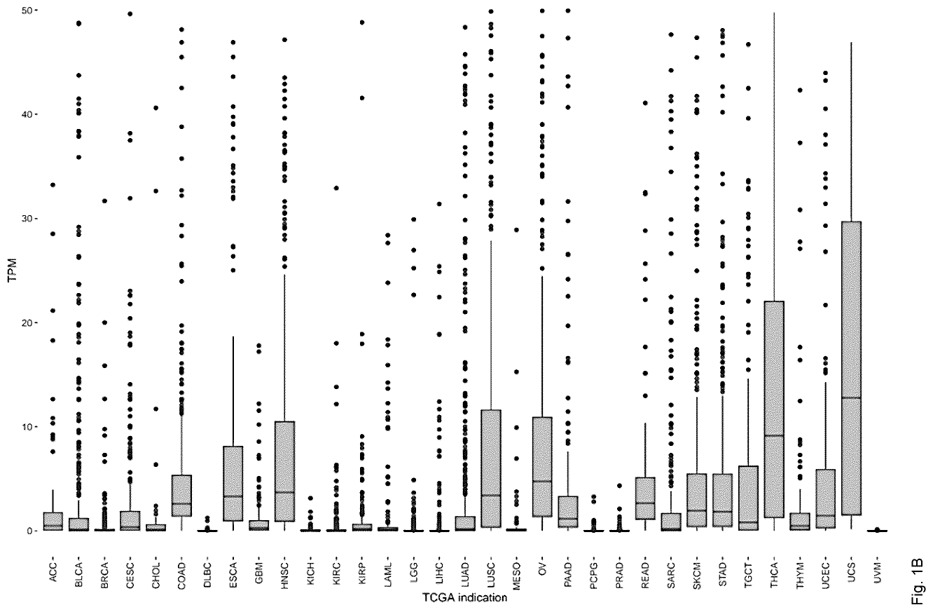

FIG. 1A-FIG. 17B and FIG. 26A-FIG. 26B. The identification of cancer specific transcripts specific to the lung squamous cancer type through the use of de novo assembly—Transcripts per million (TPM) were estimated for all transcripts and expression across healthy tissue samples from the GTEx (A panels) was compared with expression within several cancer types from TGCA (B panels).

FIG. 1A-FIG. 1B: EVA001 (SEQ ID NO.1). Cancer specificity: ESCA, HNSC, UCSC. Expression in normal: Low expression (1-2 TPMs) in tibial_nerve and colon.

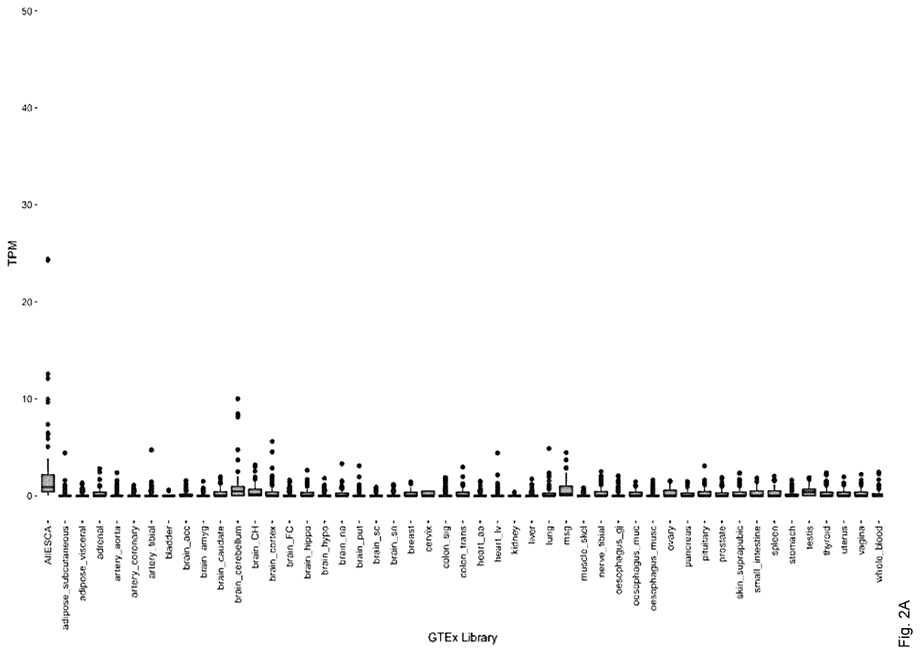

FIG. 2A-FIG. 2B. EVA002 (SEQ ID NO.2). Cancer specificity: ESCA, DLBC, PRAAD, COAD. Expression in normal: low expression (<1 TPM) in multiple organs.

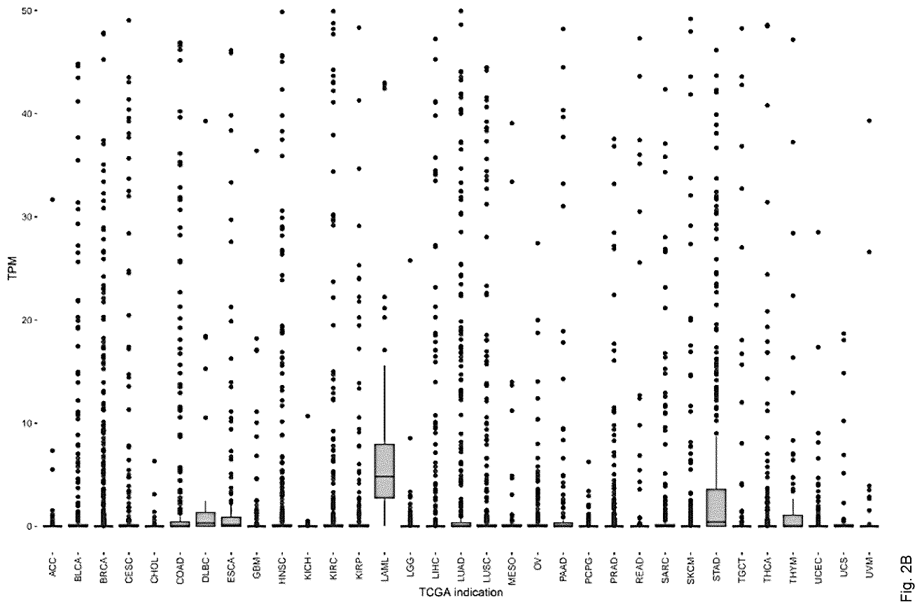

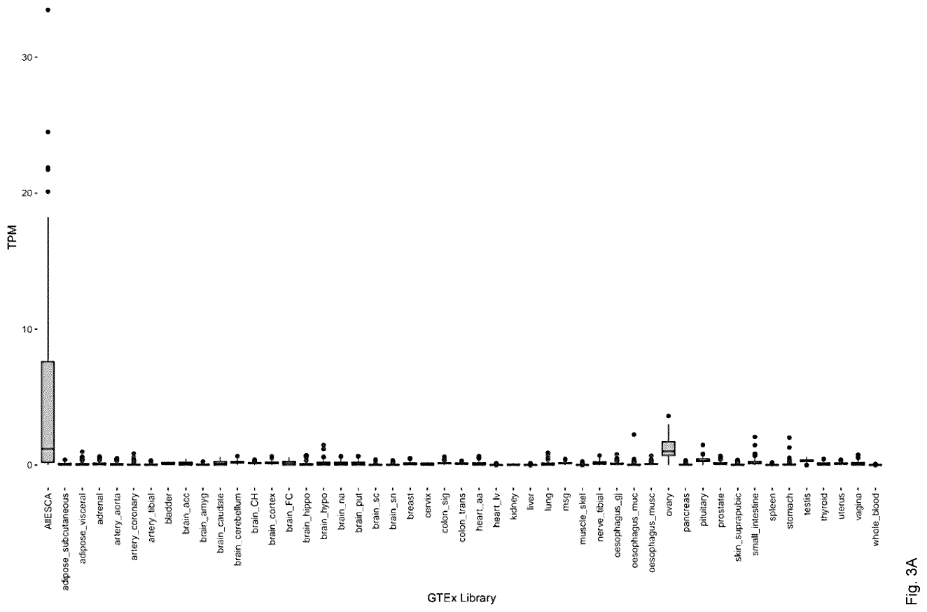

FIG. 3A-FIG. 3B. EVA003 (SEQ ID NO.3). Cancer specificity: ESCA. Expression in normal: Low ovary (2-3 TPM).

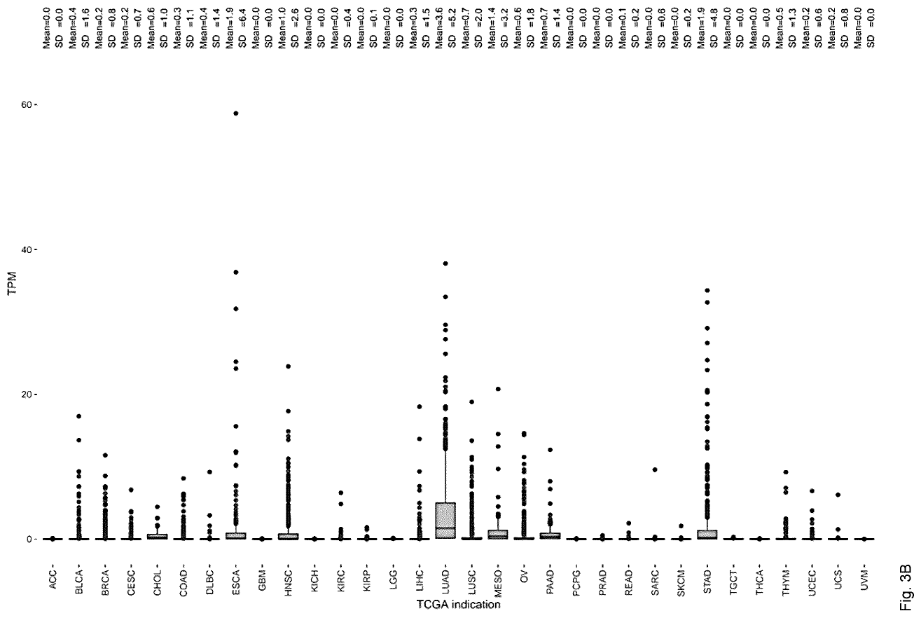

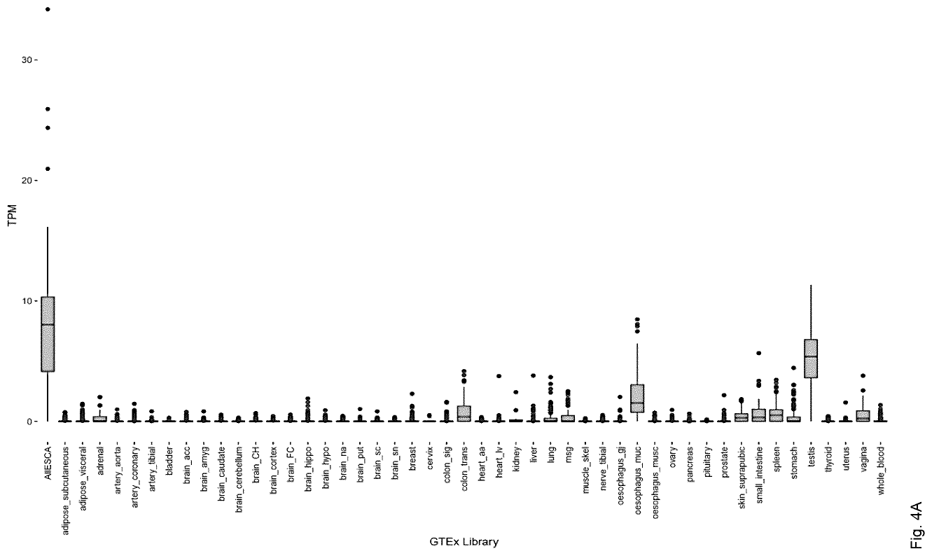

FIG. 4A-FIG. 4B. EVA004 (SEQ ID NO.4). Cancer specificity: ESCA. Expression in normal: esophagus_muc (2 TPM), colon (1 TPM), small intestine (1 TPM).

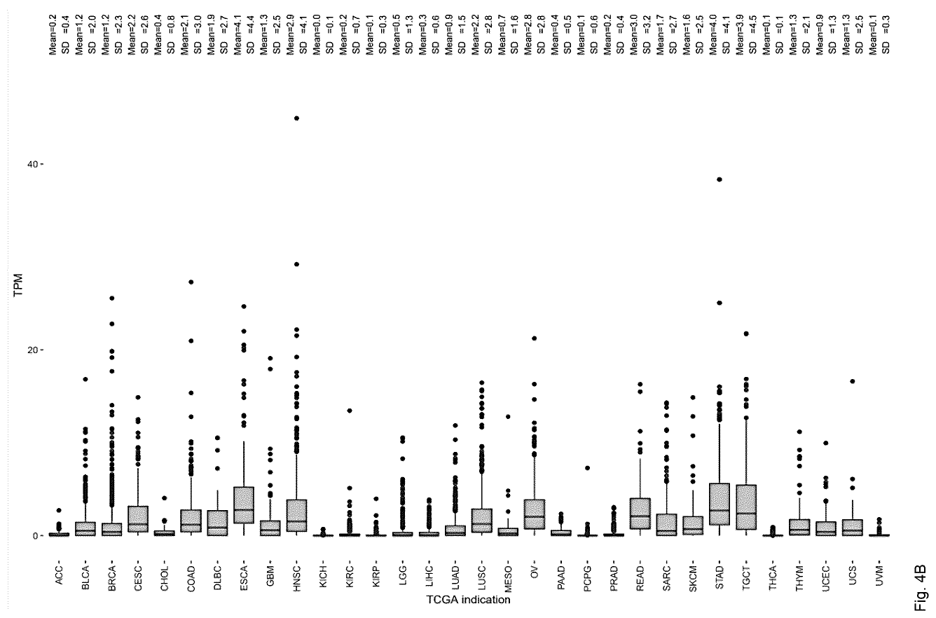

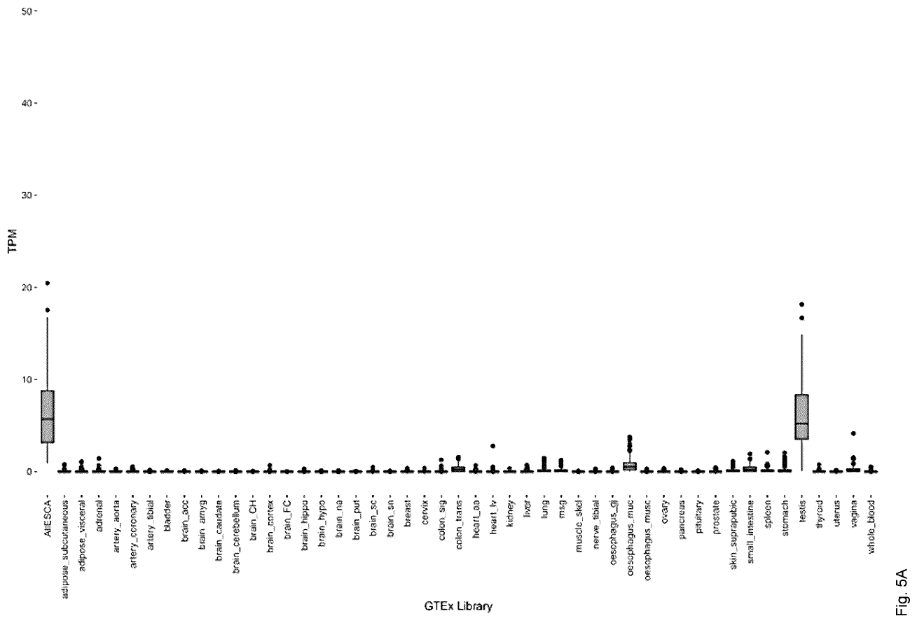

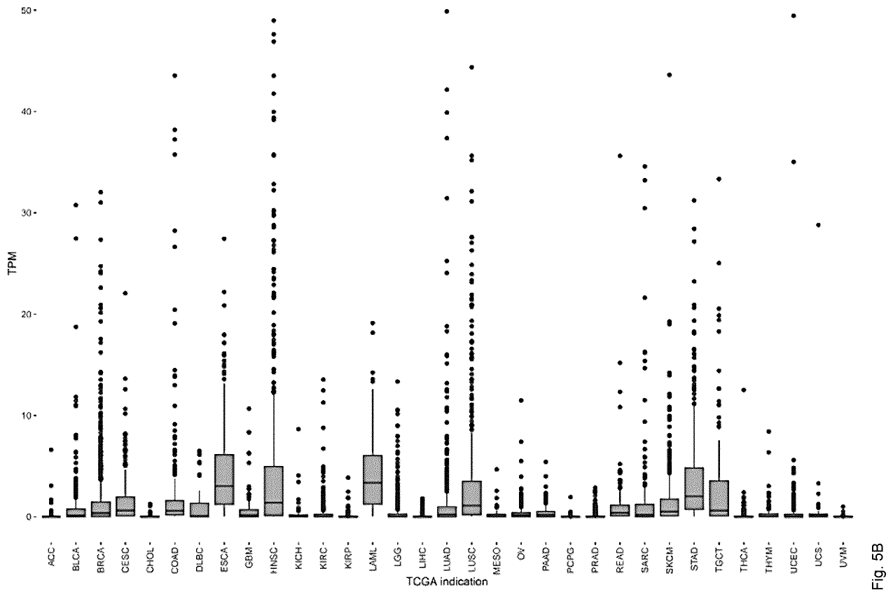

FIG. 5A-FIG. 5B. EVA005 (SEQ ID NO.5). Cancer specificity: H&N, ESCA, LALM, STAD. Expression in normal: esophageal_mun (<1 TPM), testis (˜1 TPM).

FIG. 6A-FIG. 6B. EVA006 (SEQ ID NO.6). Cancer specificity: BRCA, CHOL, ESCA, LUAD, LUSC, OV, PAAD, STAD. Expression in normal: msg, artery_coronary and lung (˜1 TPM).

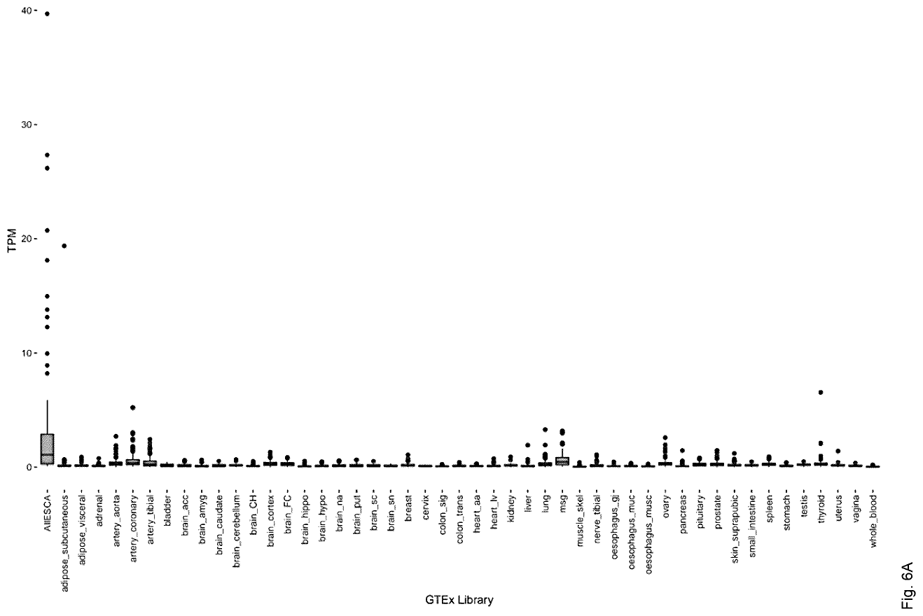

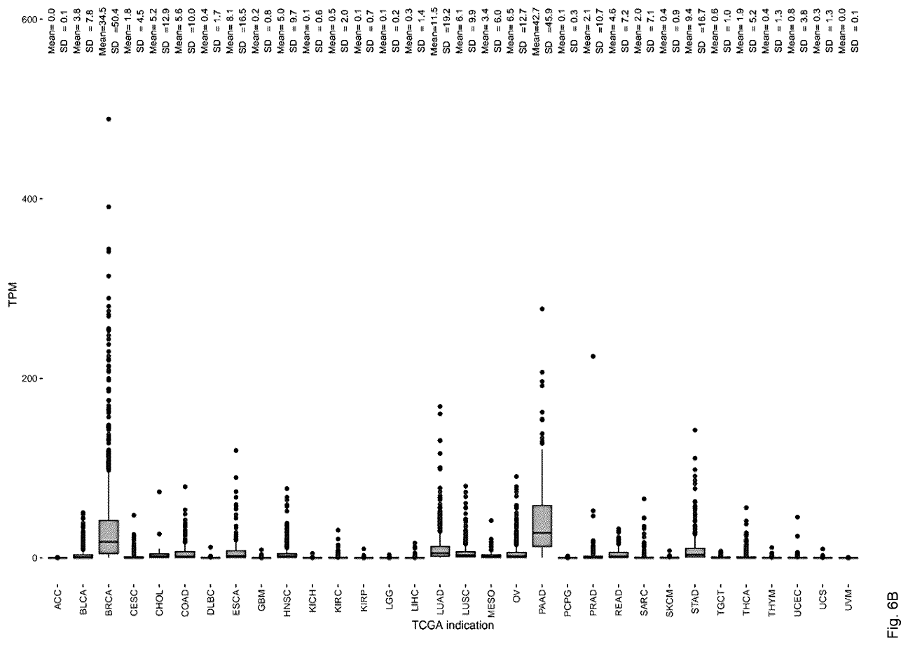

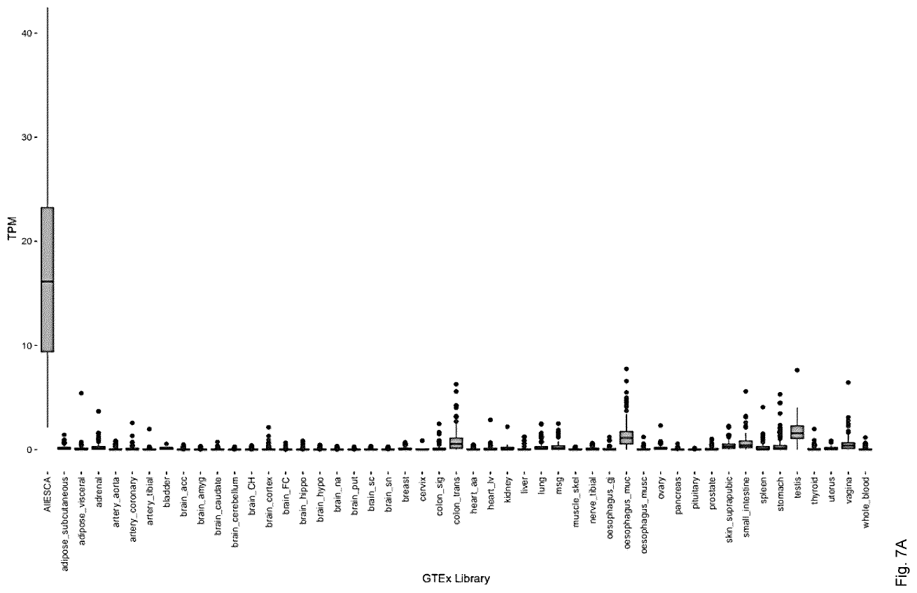

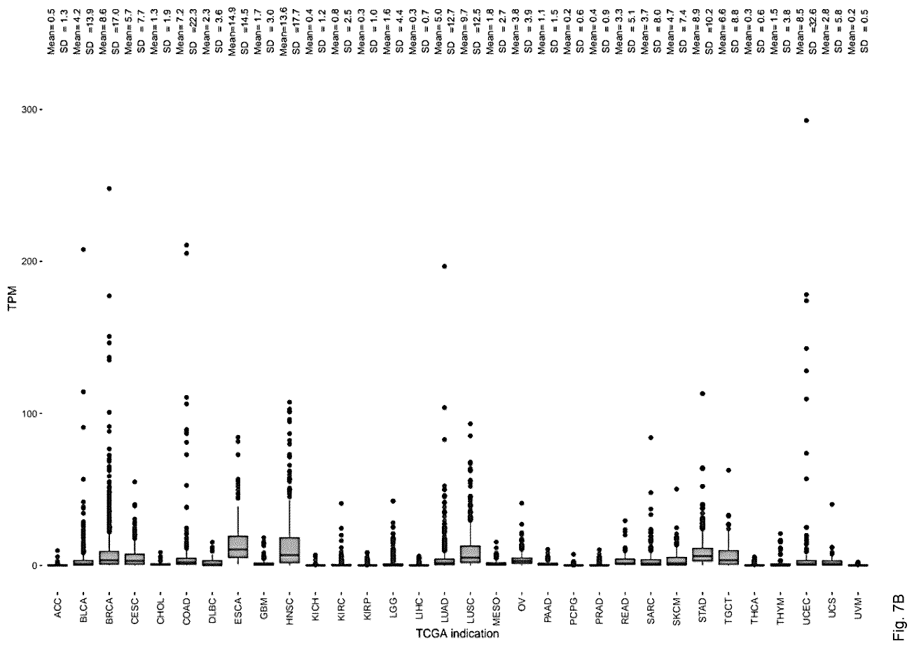

FIG. 7A-FIG. 7B. EVA007 (SEQ ID NO.7). Cancer specificity: ESCA, HNSC, LUSC. Expression in normal: esophagus_muc, colon (2-3 TPMs).

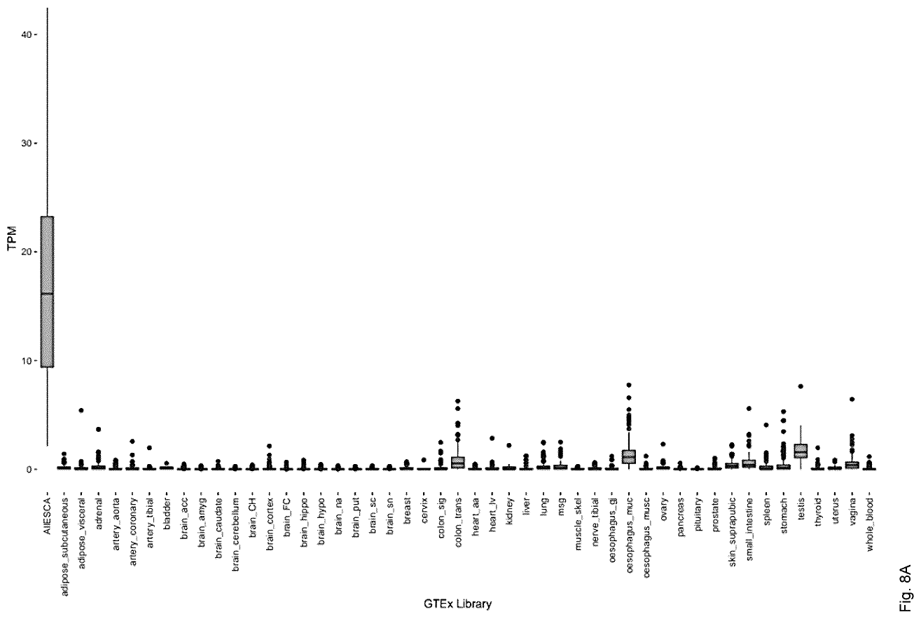

FIG. 8A-FIG. 8B. EVA008 (SEQ ID NO.8). Cancer specificity: ESCA, HNSC, LUSC. Expression in normal: esophagus_muc, colon (2-3 TPMs).

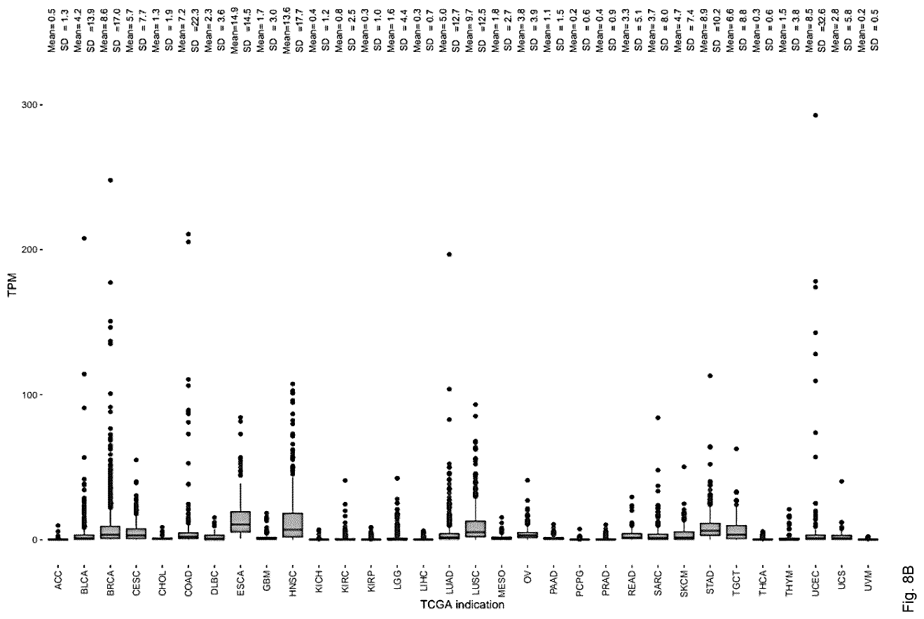

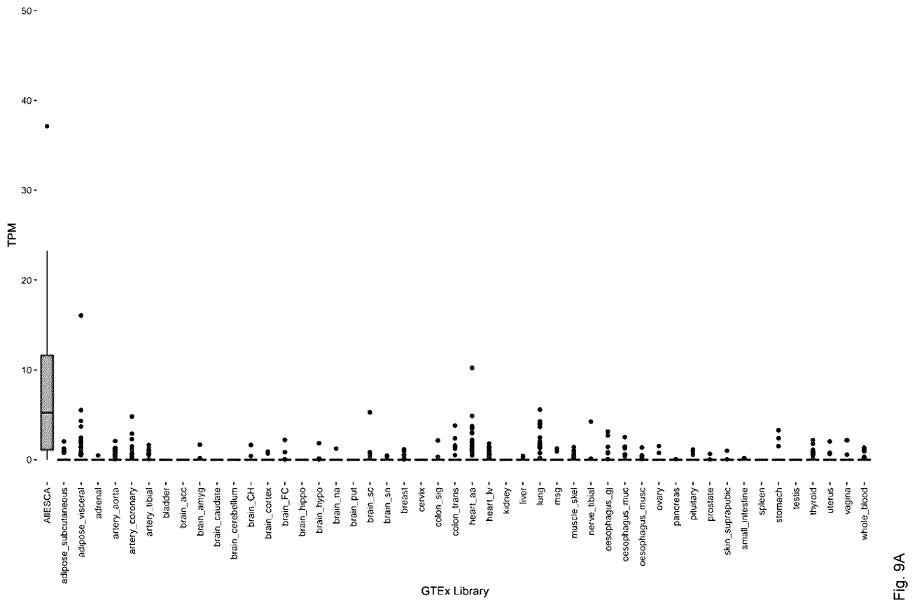

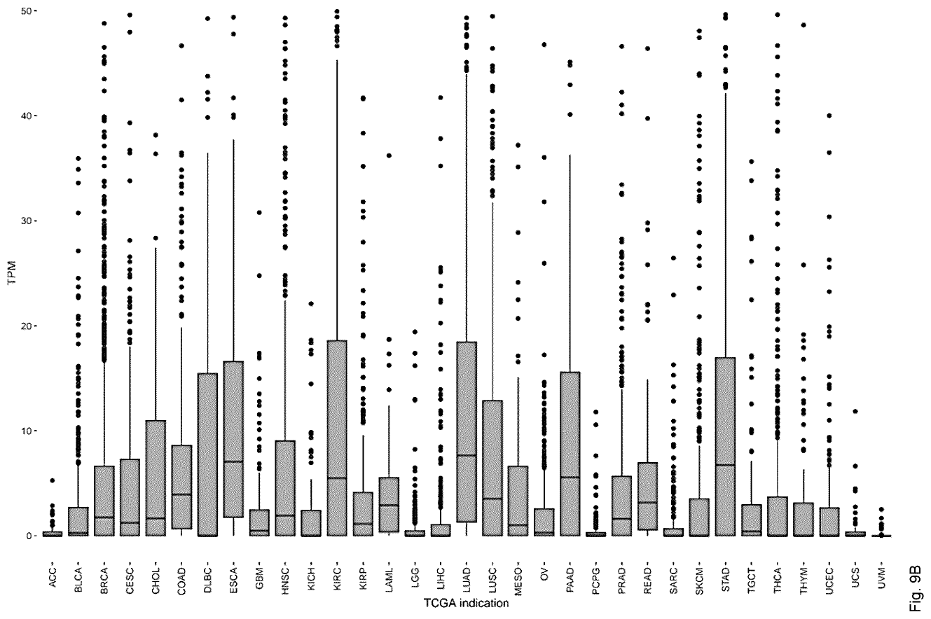

FIG. 9A-FIG. 9B. EVA009 (SEQ ID NO.9). Cancer specificity: LUAD, KIRC, ESCA. Expression in normal: Clean, no significant level of expression in normal tissues.

FIG. 10A-FIG. 10B. EVA010 (SEQ ID NO.10). HNSC, LUAD, LUSC. Expression in normal: lung, adipose, visceral, spleen, (˜1 TPM).

FIG. 11A-FIG. 11B. EVA011 (SEQ ID NO.11). Cancer specificity: STAD, MESO, LUSC and ESCA. Expression in normal: Nerve_tibial (3 TPM), stomach (2-3 TPM).

FIG. 12A-FIG. 12B. EVA012 (SEQ ID NO.12). Cancer specificity: COAD, LUAD, STAD. Expression in normal: Clean, no significant level of expression in normal tissues.

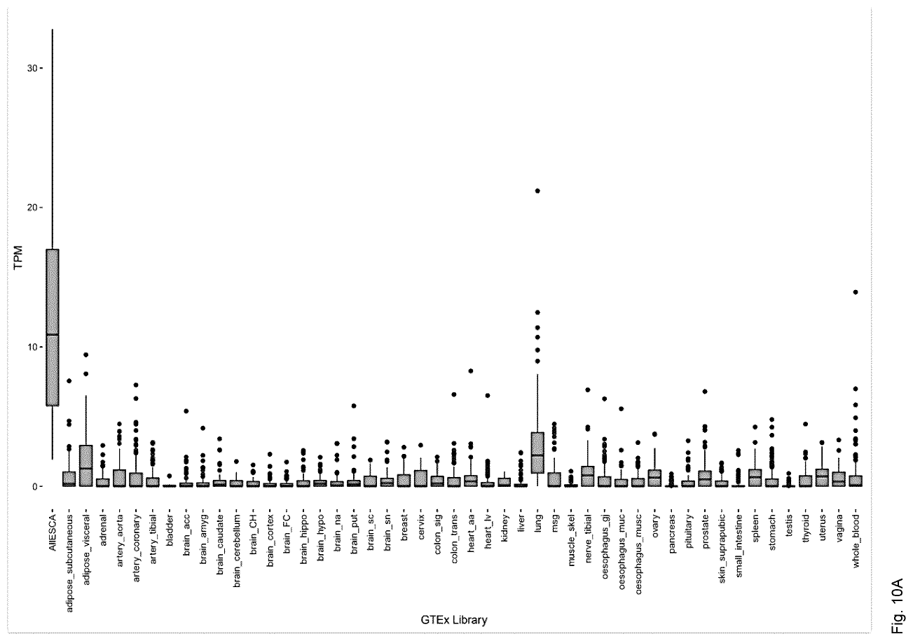

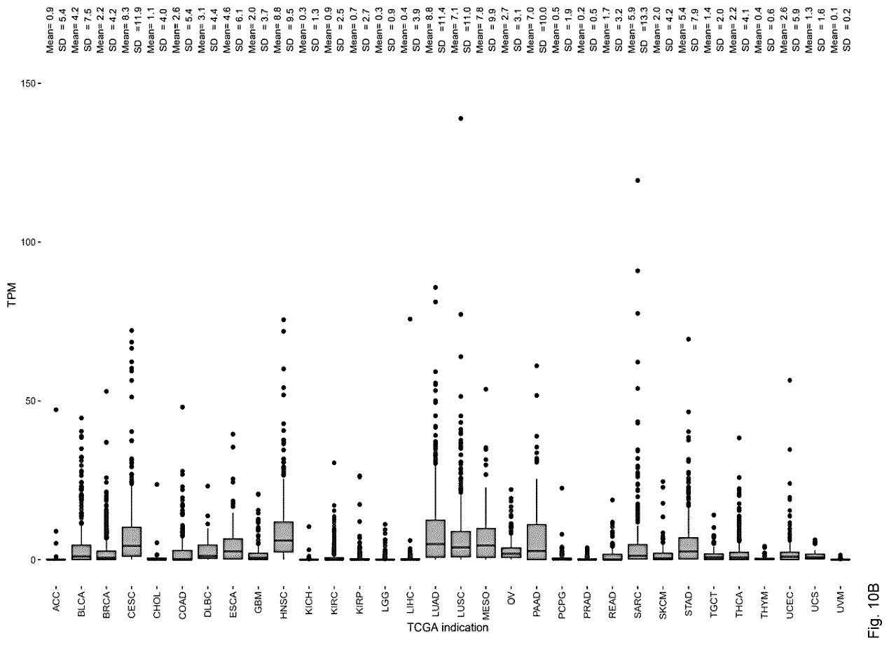

FIG. 13A-FIG. 13B. EVA013 (SEQ ID NO.13). Cancer specificity: H&N, LUSC, ESCA. Expression in normal: Esophageal_muc (˜1 TPM).

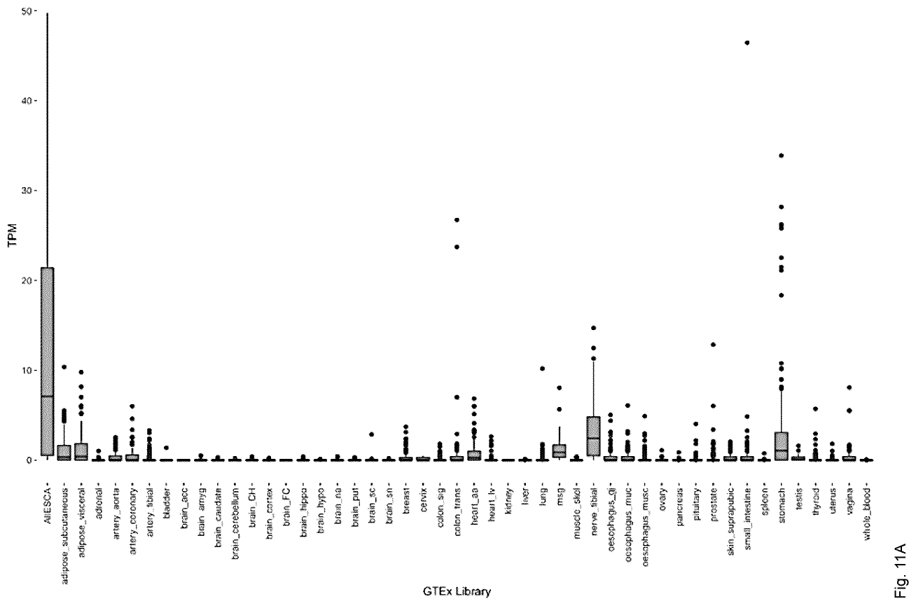

FIG. 14A-FIG. 14B. EVA014 (SEQ ID NO.14). Cancer specificity: H&N, LUSC, ESCA. Expression in normal: Esophageal_muc (˜1 TPM).

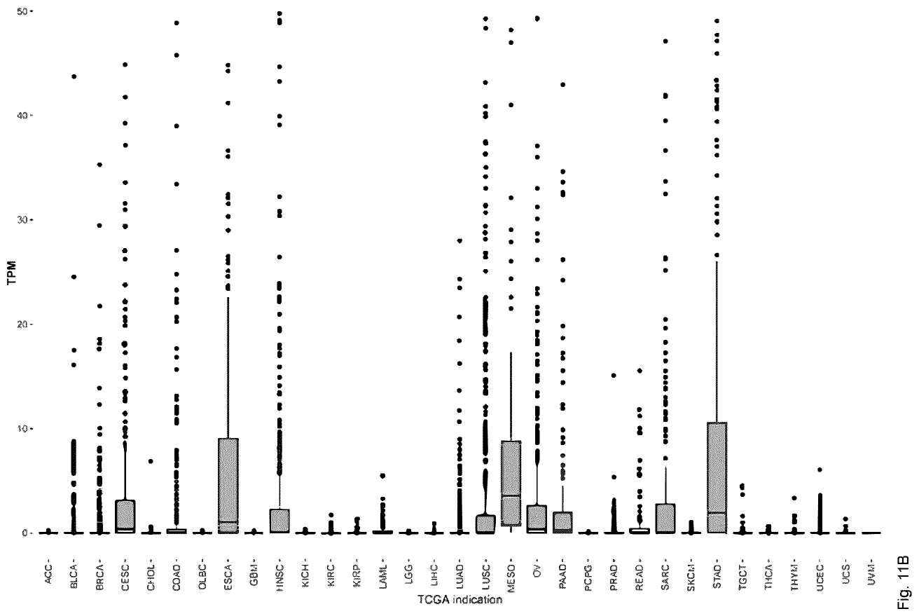

FIG. 15A-FIG. 15B. EVA015 (SEQ ID NO.15). Cancer specificity: LCA, CESC, COAD, ESCA, HNSC, LUSC, OV, READ, SARC, STAD, TGCT. Expression in normal: Esophagus, testis, colon_trans (3-4 TPM).

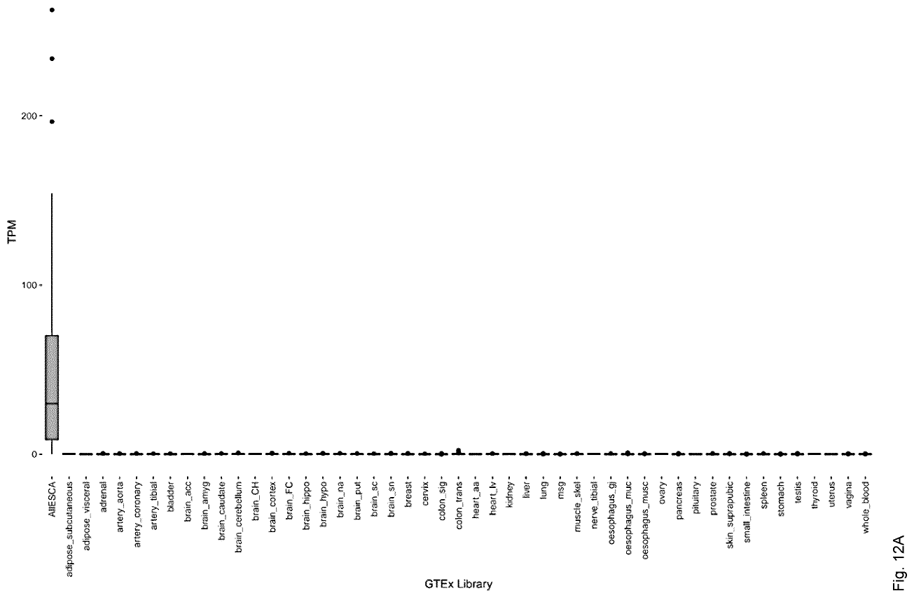

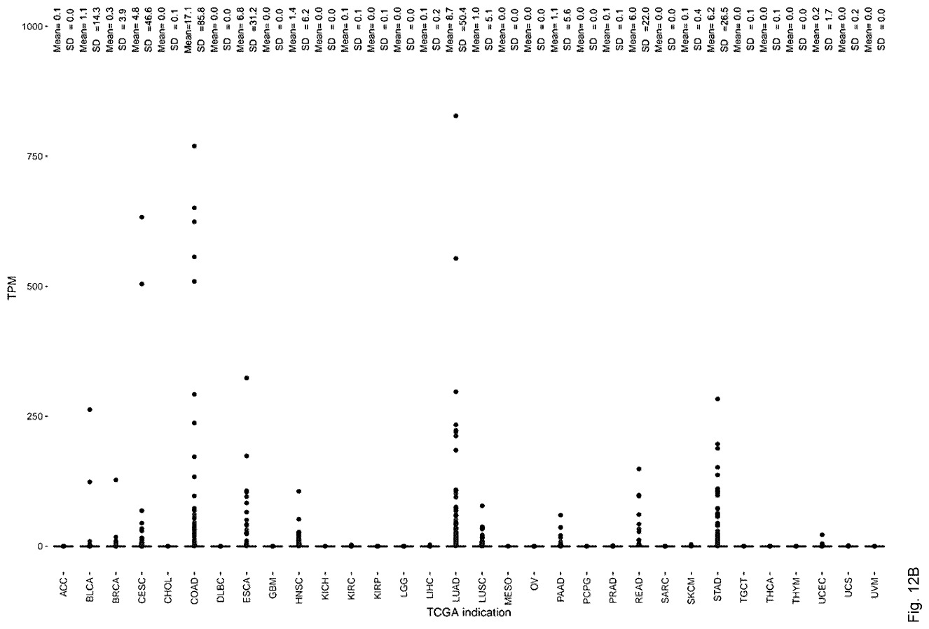

FIG. 16A-FIG. 16B. EVA016 (SEQ ID NO.16). Cancer specificity: ESCA. Expression in normal: Clean, no significant level of expression in normal tissues.

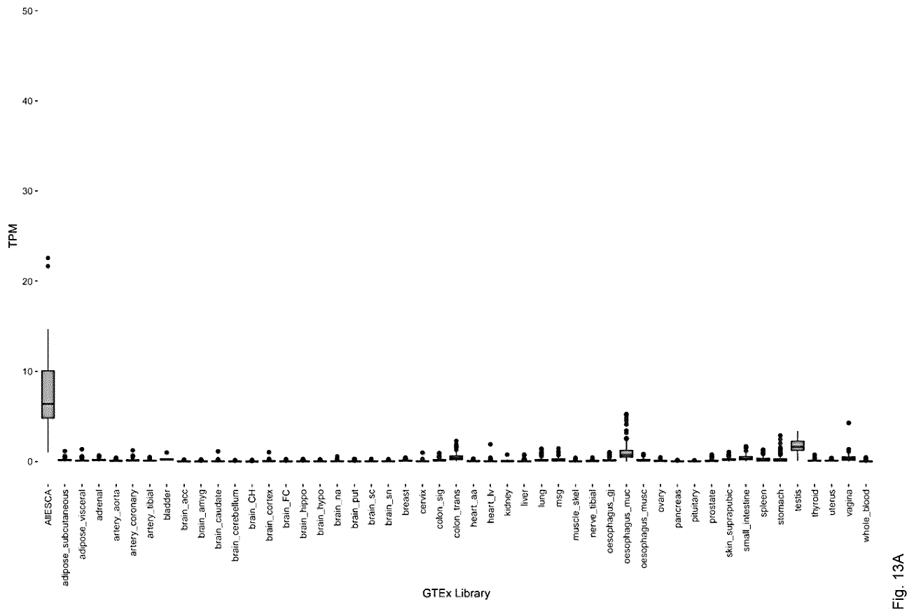

FIG. 17A-FIG. 17B. EVA017 (SEQ ID NO.17). HNSC, CESC, LUSC. Expression in normal: esophagus_muc, spleen, thyroid (˜1 TPM).

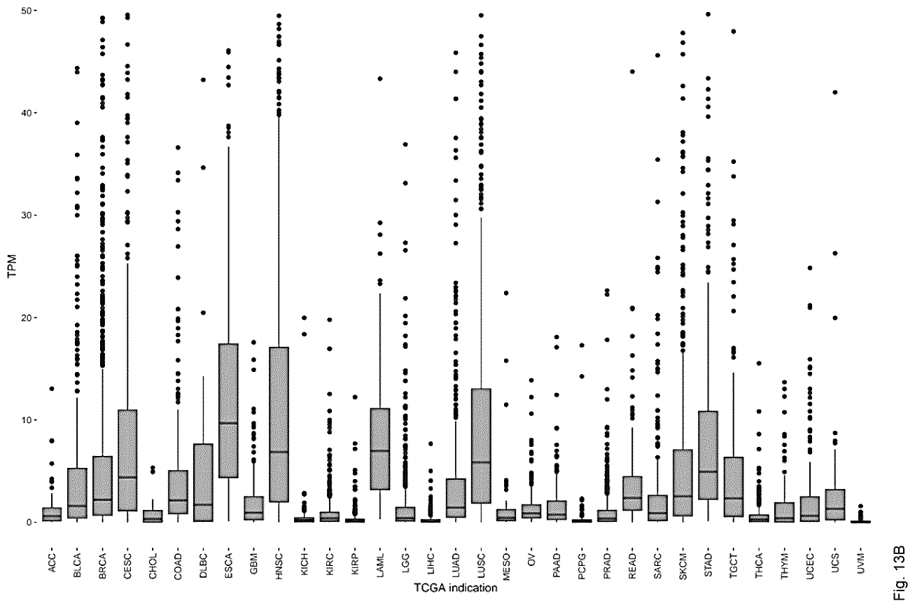

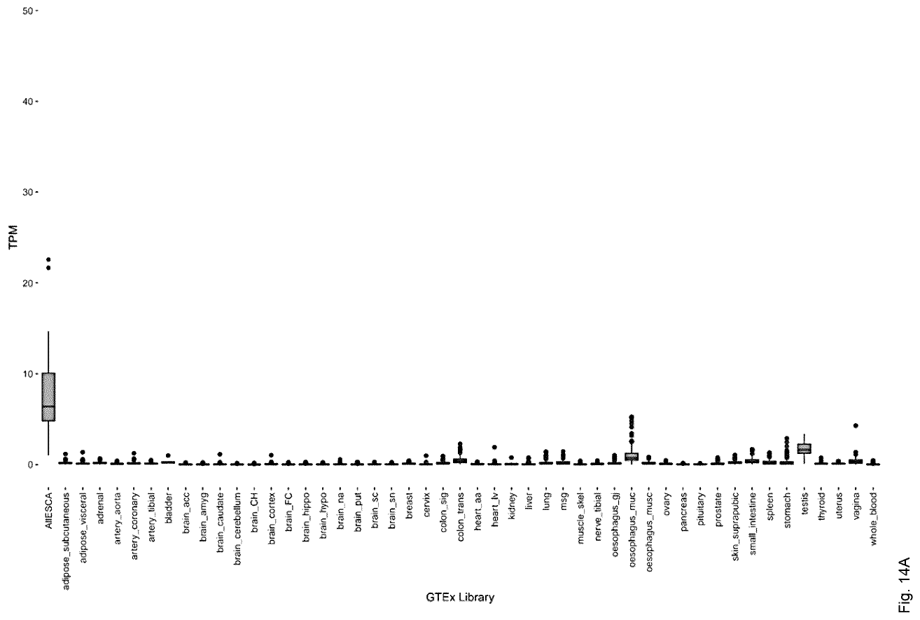

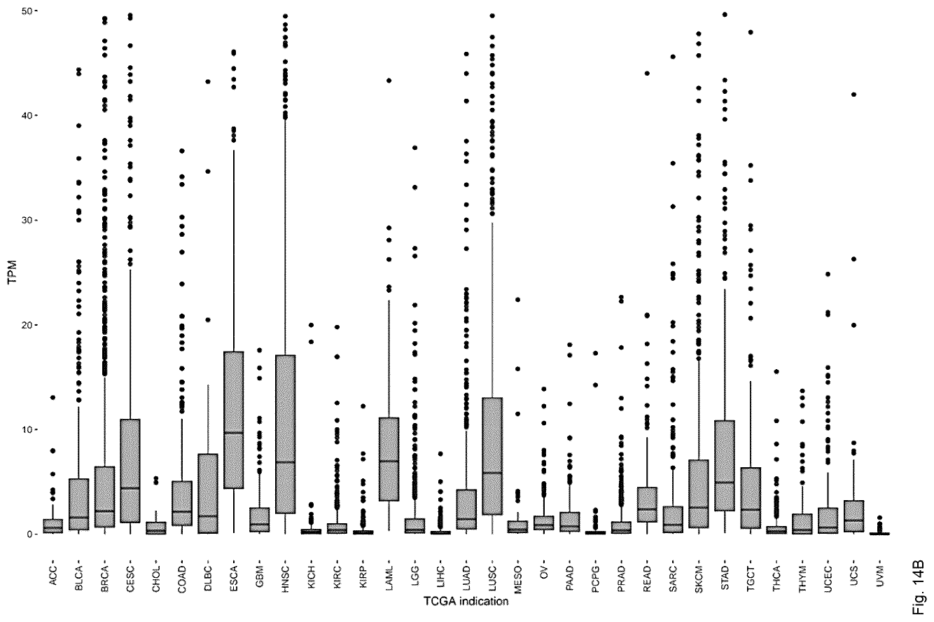

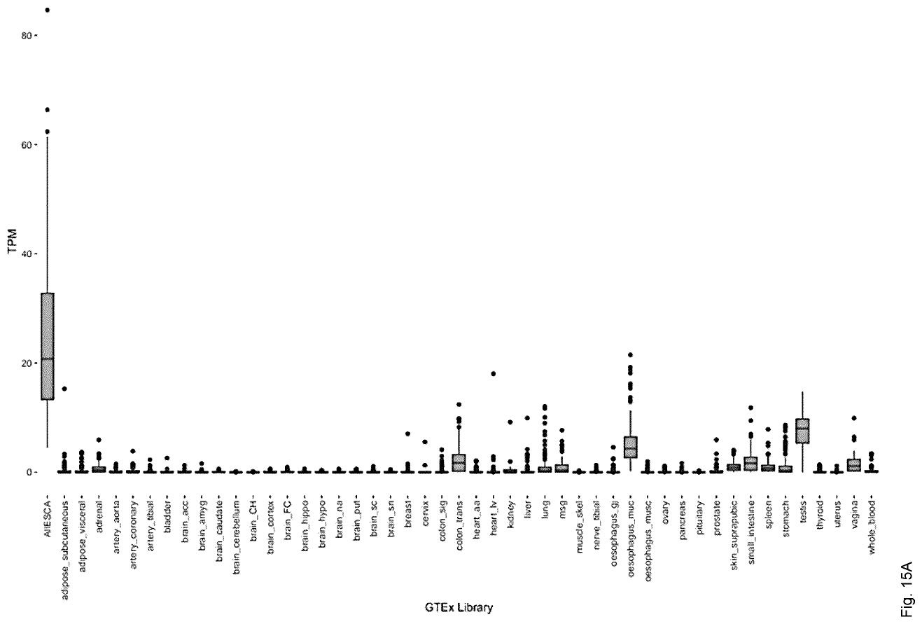

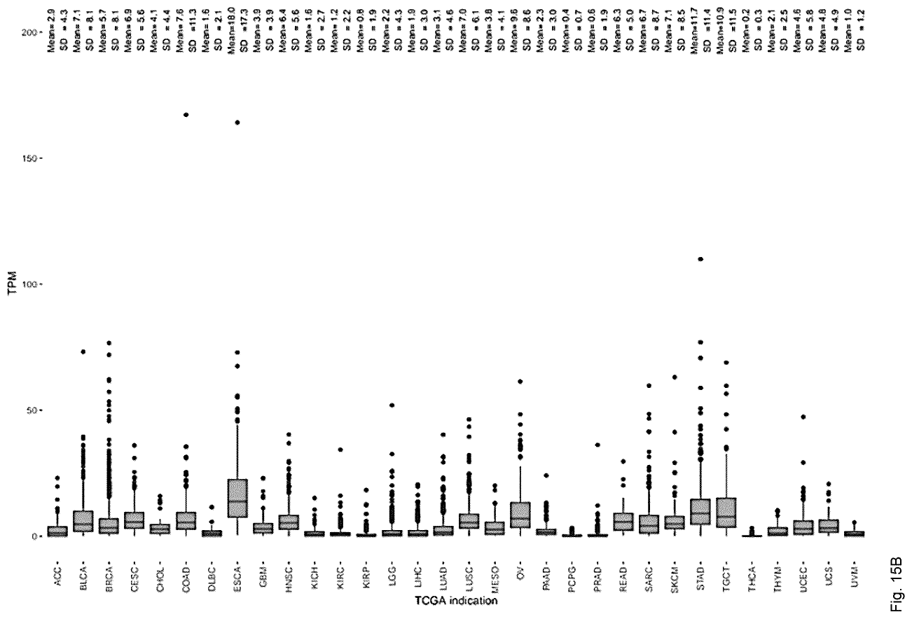

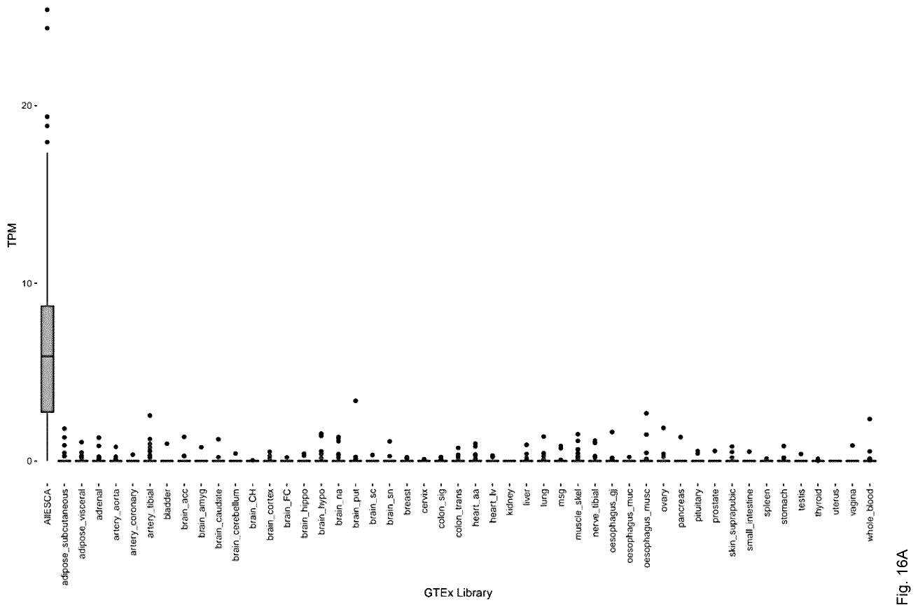

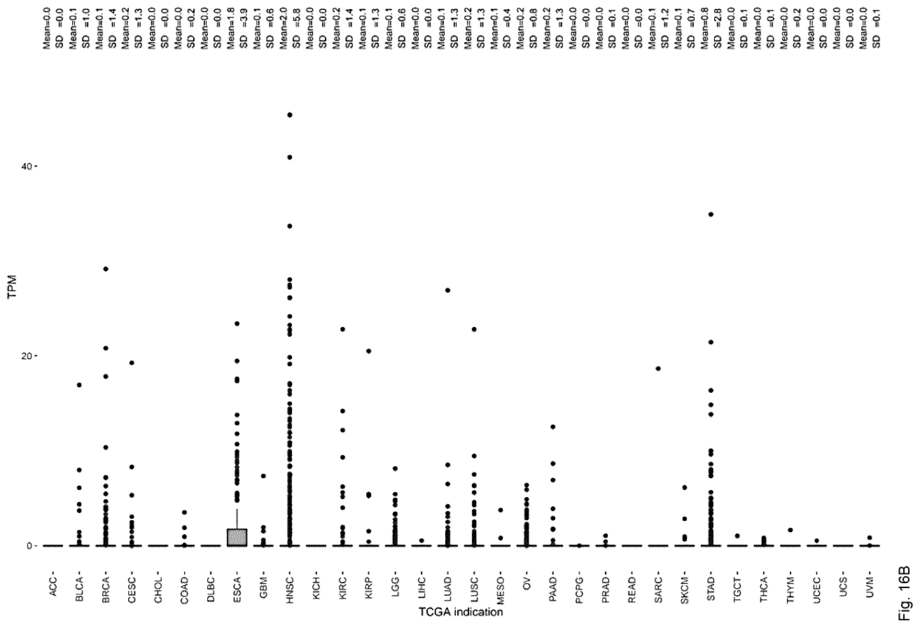

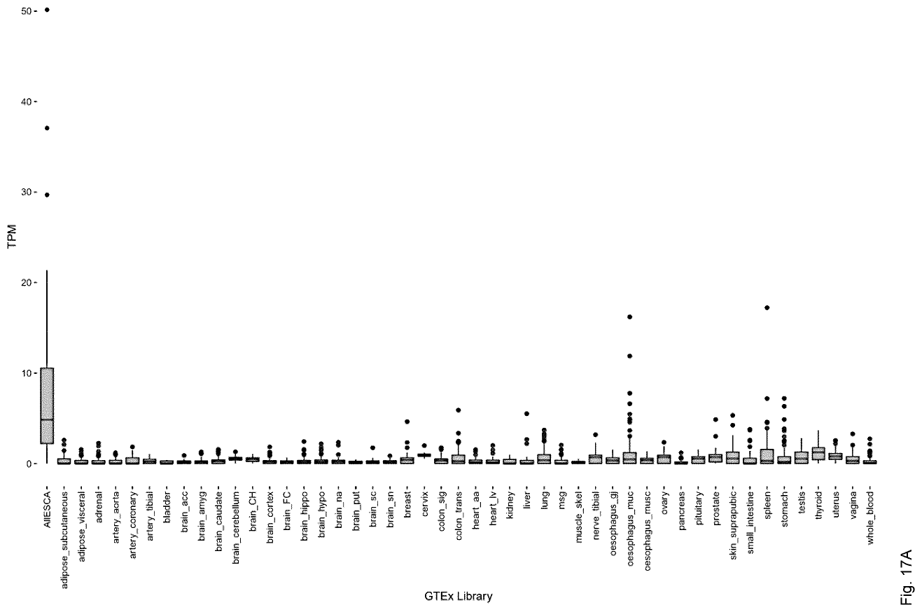

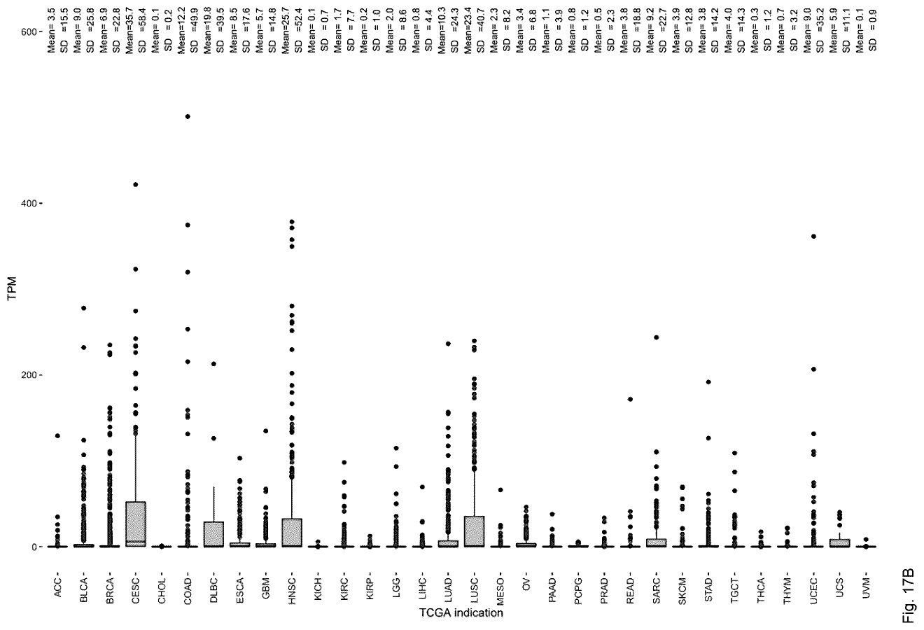

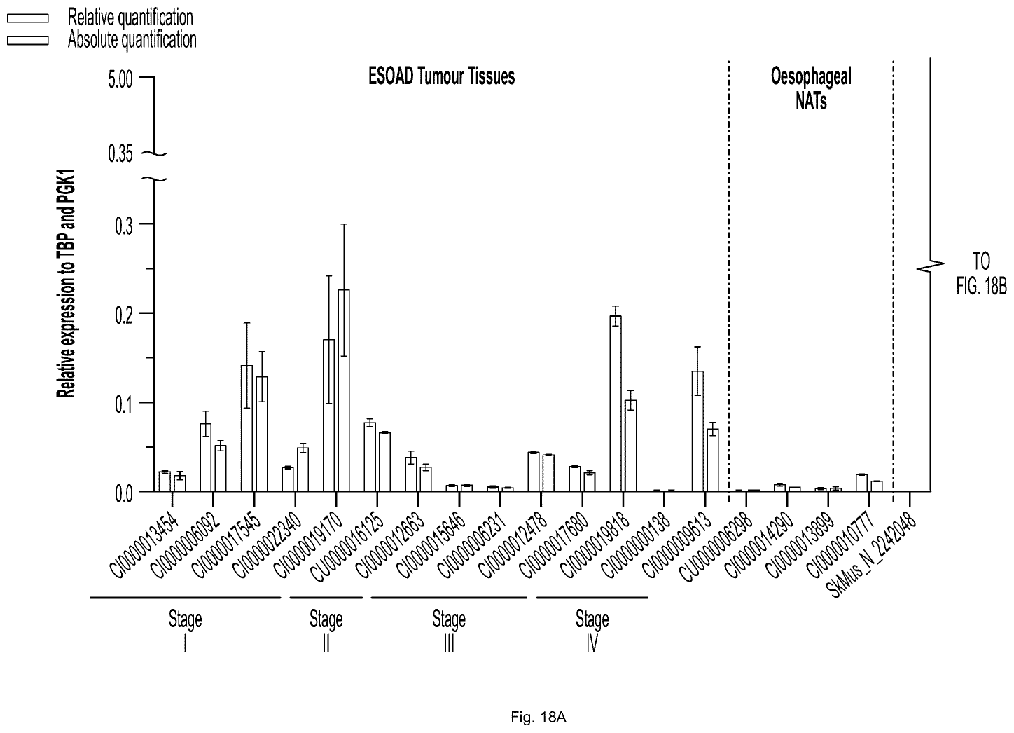

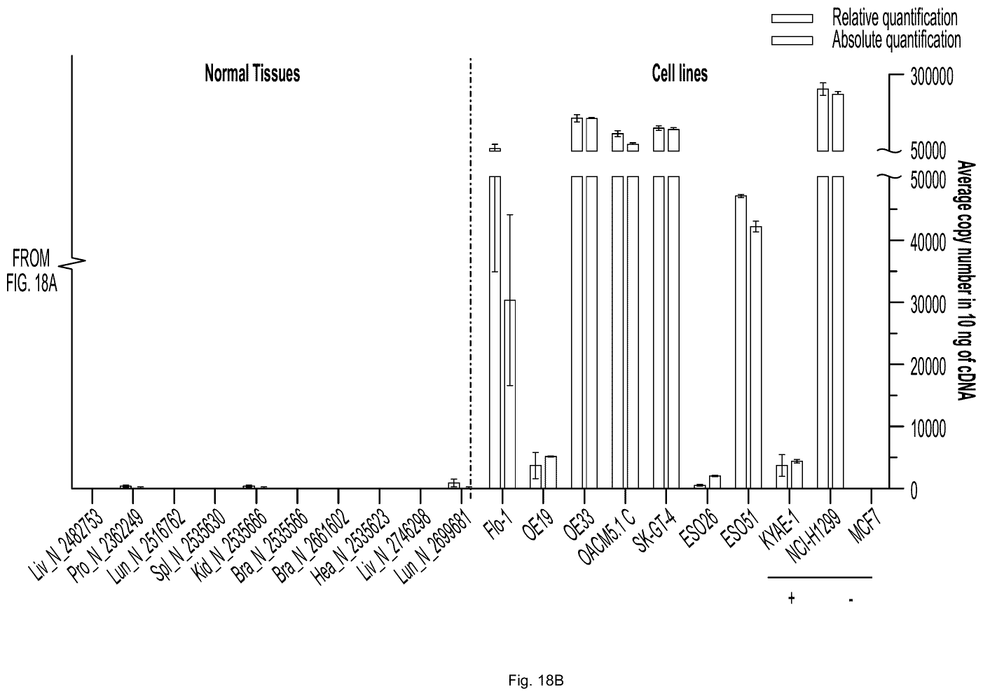

FIG. 18A-FIG. 18B: EVA001 transcript expression by RT-qPCR in EAC esophageal adenocarcinoma (denoted ESOAD in the figure) tissues and EAC cell lines. Data shows a low-level expression in the majority of LUSC tissues (8/11) and at a mid-level in the majority of NSCLC cell lines (11/13) relative to reference genes TBP and PGK1 which are expressed at a consistent level across EAC tissues. The following cell lines are shown: Flo-1 esophageal adenocarcinoma (EAC) cell, OE19 (EAC) cell, OE33 (EAC) cell, OACM5.1C (EAC) cell, SKGT4 distal esophagus EAC, ESO26 distal esophagus EAC, ESO51 distal esophagus EAC, KYAE-1 distal esophagus EAC. Positive control cell line is NCI-H1299 lung carcinoma cell known to express the EVA001 transcript. Negative control cell line is MCF7 breast cancer cell line, known not to express EVA001. EVA001 has high prevalence in EAC with expression observed in 11/14 tumour tissue samples, and 7/7 tumour cell lines. In most cases, when present, EVA001 is expressed at high levels relative to TBP and PGK1 reference genes. (NAT=normal adjacent tissue)

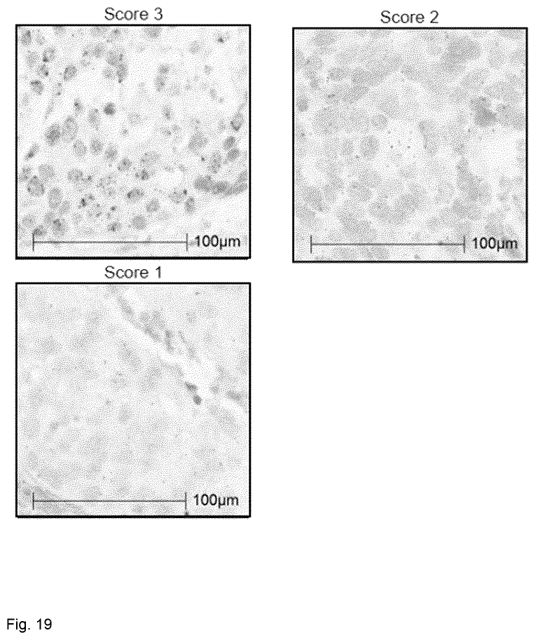

FIG. 19: Representative examples of RNAscope scoring levels as assigned by microscopic observation.

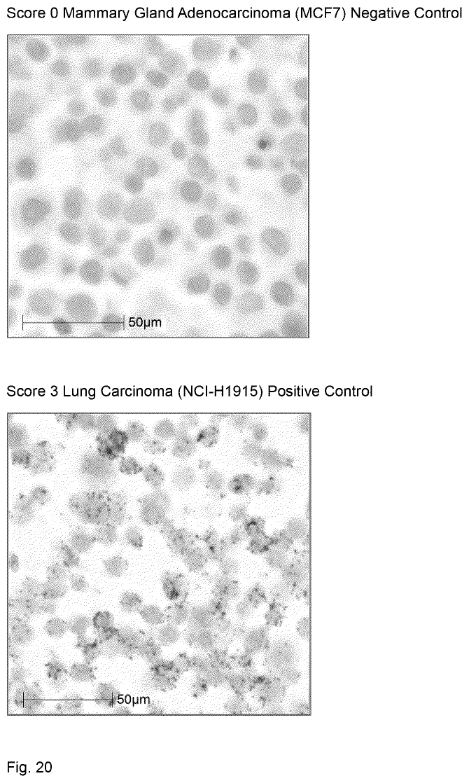

FIG. 20: EVA001 transcript expression detected by RNAscope probe EVA001-20zz-RNAscope in Esophageal Adenocarcinoma (EAC) tissues, and in control tissues known by prior sequencing analysis to express or not express either transcript sequence. EVA001-20zz-RNAscope binds within the region of SEQ ID NO. 17 which includes the sequence encoding peptide SEQ ID NO. 18.

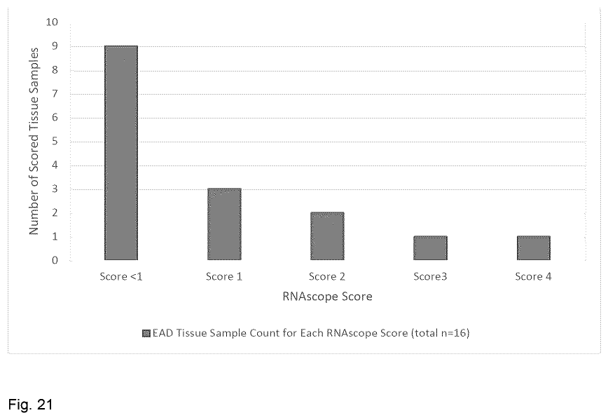

FIG. 21: EVA001 transcript expression in EAC tissues detected by RNAscope probe EVA001-20zz-RNAscope. The expression (score≥1) was observed in 7/16 EAC tissues (44%) and positivity was observed across almost all tumour stages tested.

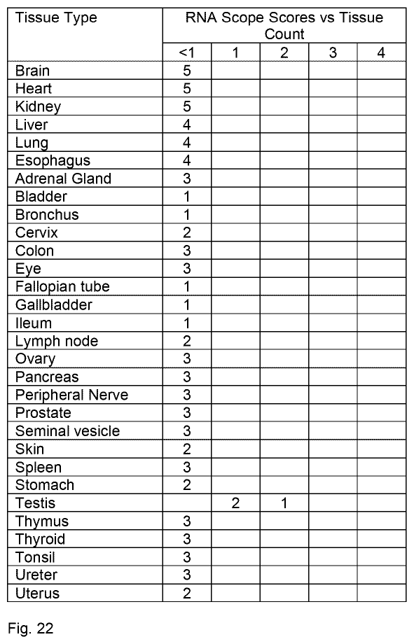

FIG. 22: EVA001 transcript expression in normal tissues by RNAscope probe EVA001-20zz-RNAscope. EVA001 expression (score<1) was observed generally across normal tissues: Values>1 were seen in normal tissue samples of testis only (3/3 tissues).

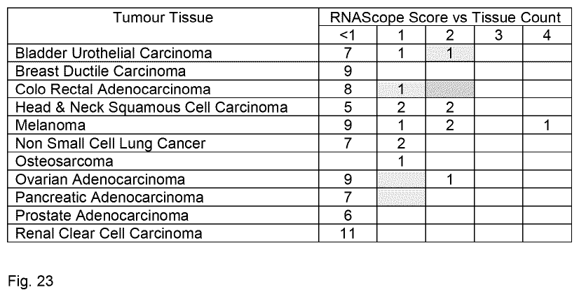

FIG. 23: EVA001 transcript expression in selected tumour tissues by RNAscope probe EVA001-20zz-RNAscope. EVA001 expression was observed in 7/11 generalised tumour tissue types.

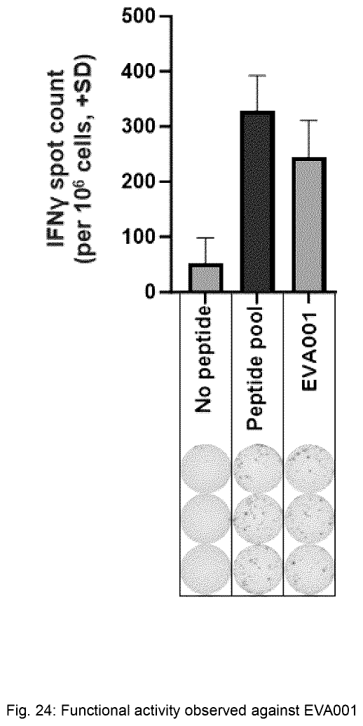

FIG. 24: IFN gamma CD8 T-cell responses from normal blood donors to the HLA restricted CST antigen peptide SEQ ID NOs:18 (EVA001)

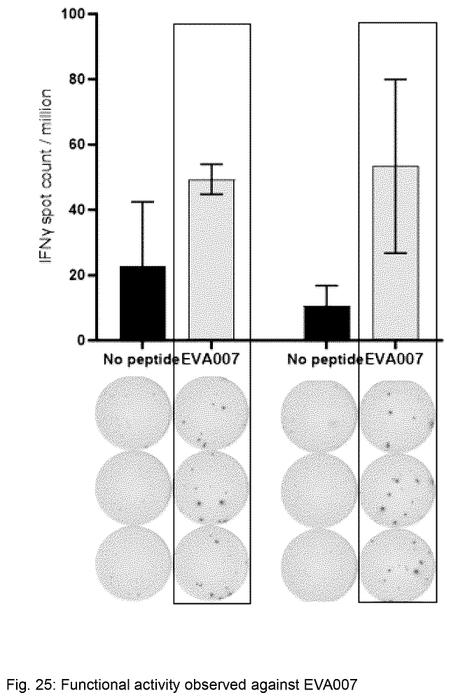

FIG. 25: IFN gamma CD8 T-cell responses from normal blood donors to the HLA restricted CST antigen peptide SEQ ID NOs:26 (EVA007).

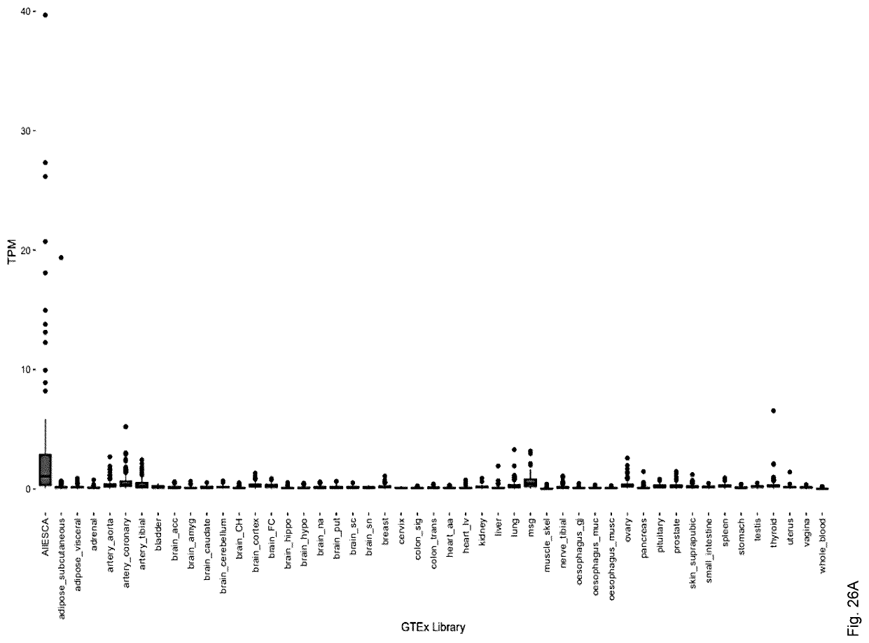

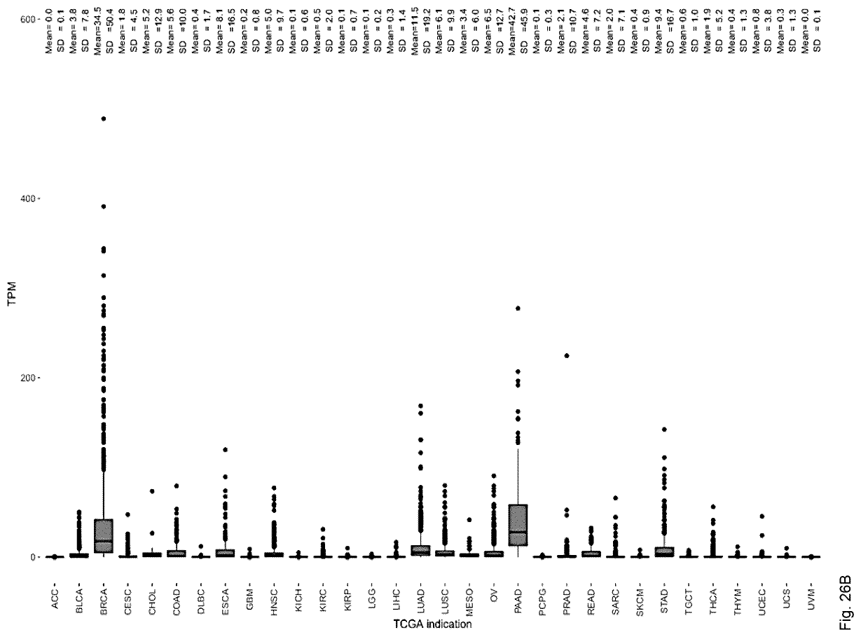

FIG. 26A-FIG. 26B. EVA018 (SEQ ID NO.57). Cancer specificity: BRCA, ESCA, LUAD, PAAD, STAD. Expression in normal: thyroid (˜1 TPM).

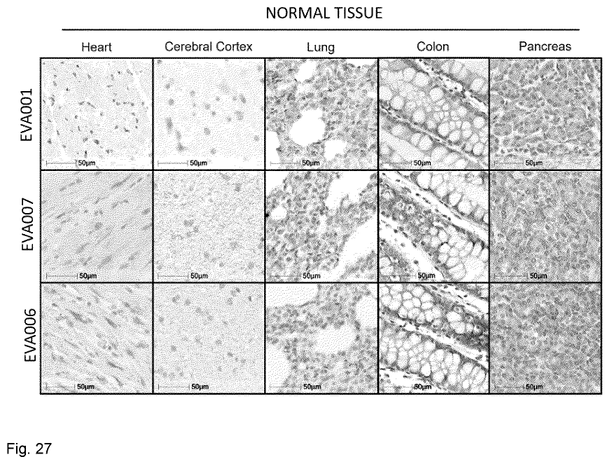

FIG. 27. EVA001, EVA006 and EVA007 transcript expression in normal tissues by RNAscope probes: EVA001-20zz-RNAscope, EVA006-20zz-RNAscope, EVA007-20zz-RNAscope.

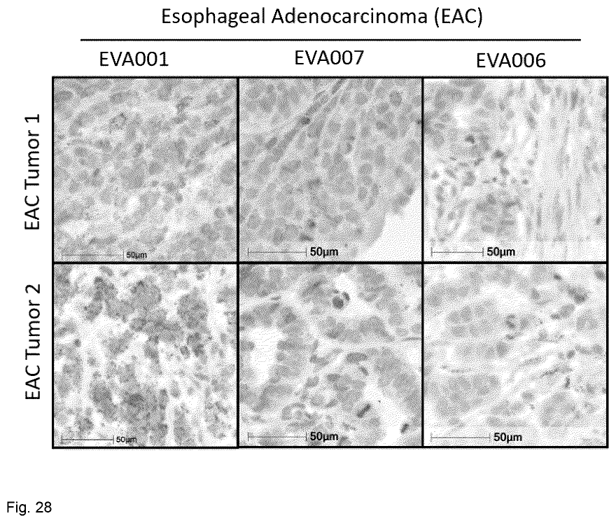

FIG. 28. EVA001, EVA006 and EVA007 transcript expression in Esophageal Adenocarcinoma (EAC) tissues by RNAscope probes: EVA001-20zz-RNAscope, EVA006-20zz-RNAscope, EVA007-20zz-RNAscope.

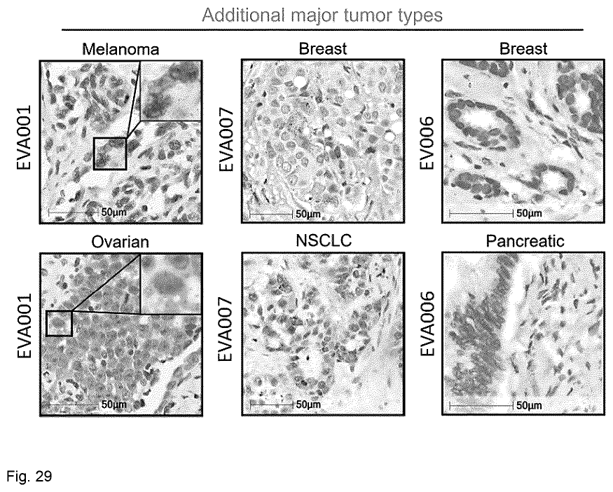

FIG. 29. EVA001, EVA006 and EVA007 transcript expression in further tumor tissues by RNAscope probes: EVA001-20zz-RNAscope, EVA006-20zz-RNAscope, EVA007-20zz-RNAscope.

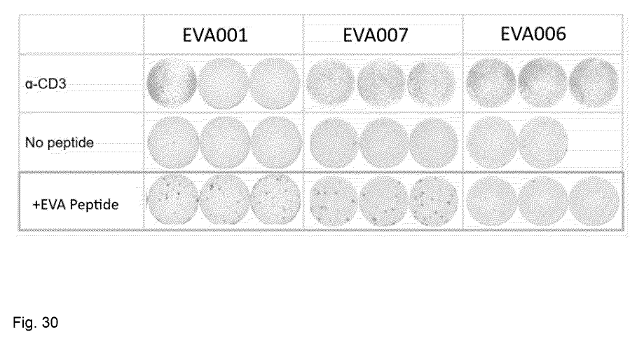

FIG. 30: IFN gamma CD8 T-cell responses from normal blood donors to the HLA restricted CST antigen peptide, SEQ ID NOs:18 (EVA001), SEQ ID NOS: 26 (EVA007) and SEQ ID NOs:25 (EVA006).

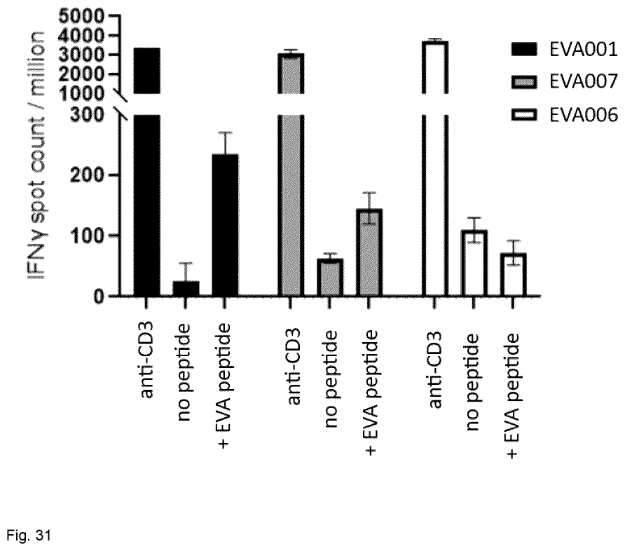

FIG. 31: Bar chart report of IFN gamma CD8 T-cell responses from normal blood donors to the HLA restricted CST antigen peptide, SEQ ID NOs:18 (EVA001), SEQ ID NOs:26 (EVA007) and SEQ ID NOs:25 (EVA006).

FIG. 32A-FIG. 32B: EVA015 (SEQ ID NO.15), transcript expression by RT-qPCR screening in oesophageal adenocarcinoma tissues.

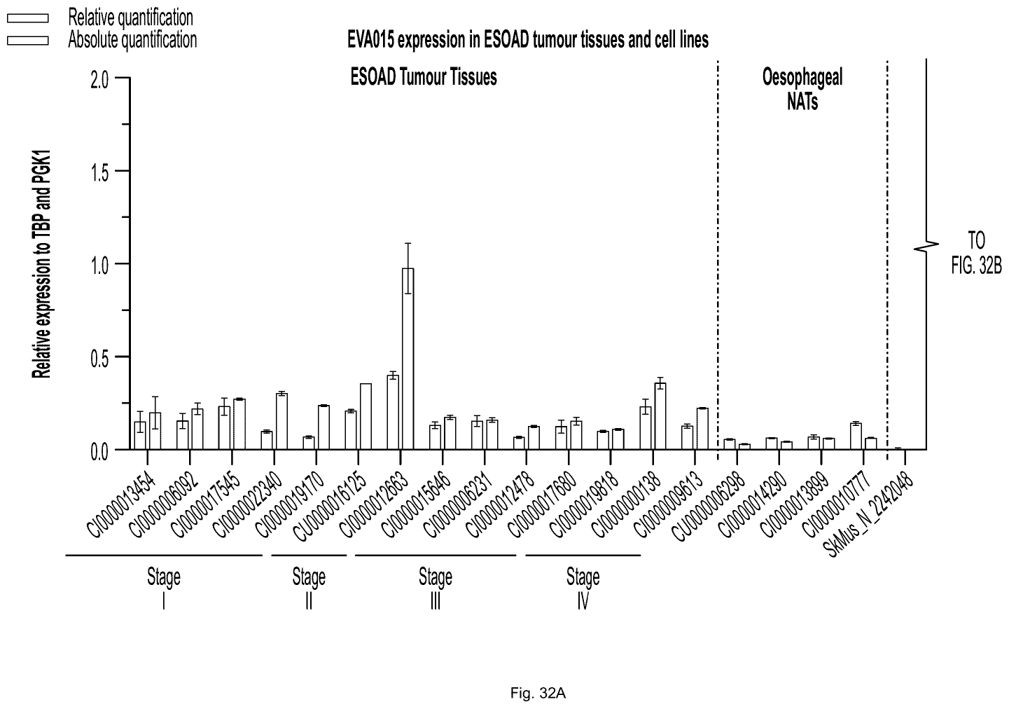

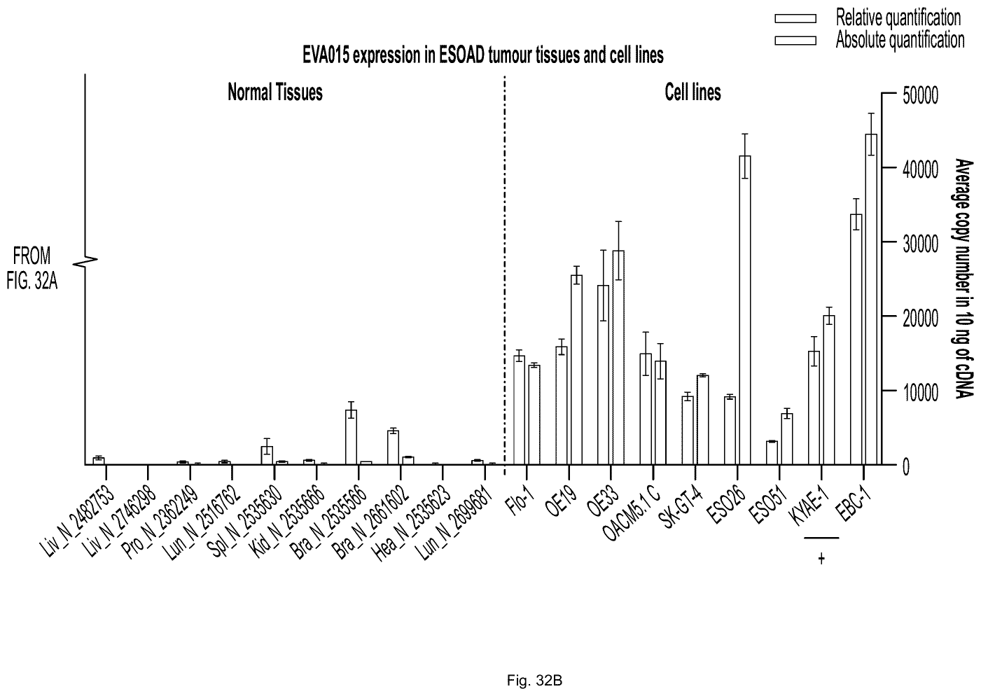

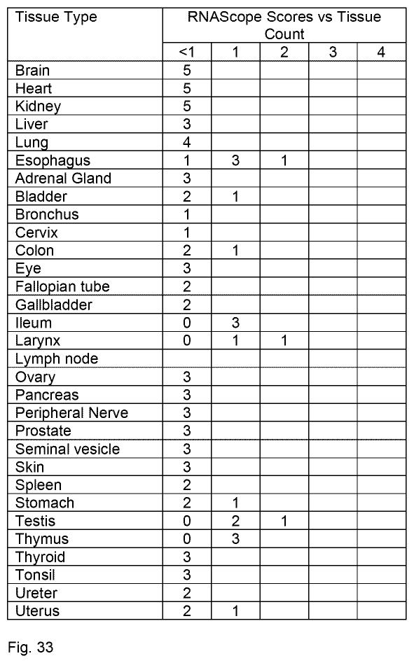

FIG. 33: EVA015 transcript expression in normal tissues by RNAscope probe: EVA015-20zz-RNAscope.

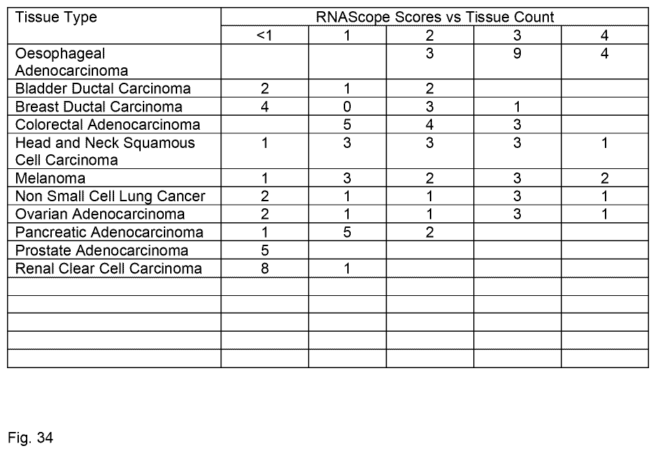

FIG. 34: EVA015 transcript expression in tumour tissues by RNAscope probe: EVA015-20zz-RNAscope.

FIG. 35A-FIG. 35B: EVA004 (SEQ ID NO.4), transcript expression by RT-qPCR screening in oesophageal adenocarcinoma tissues.

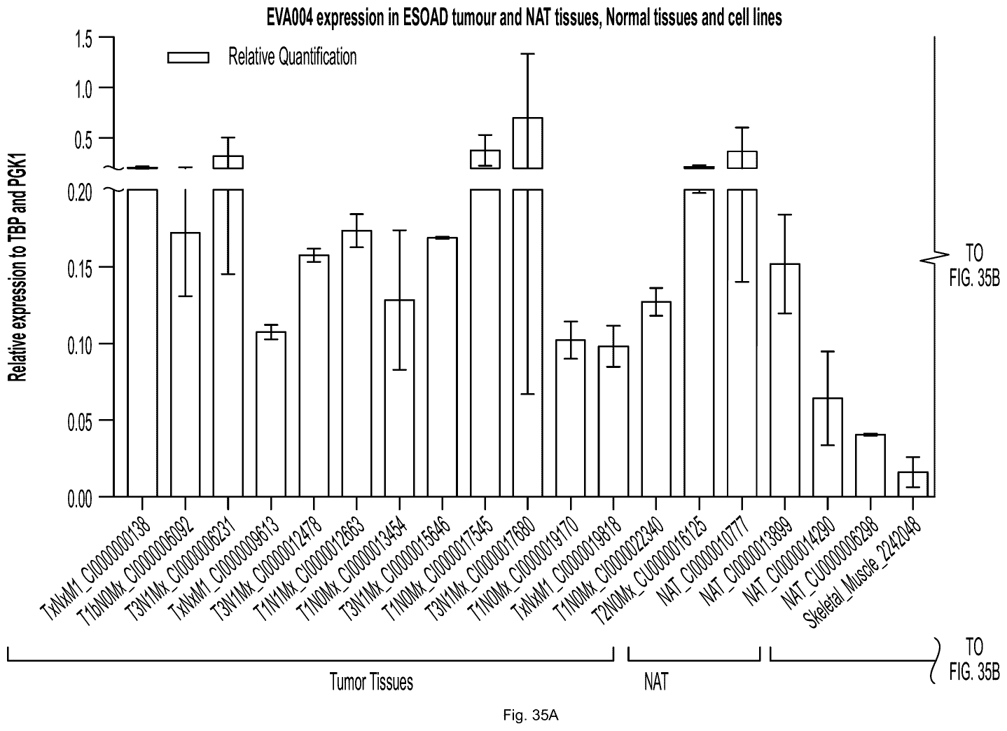

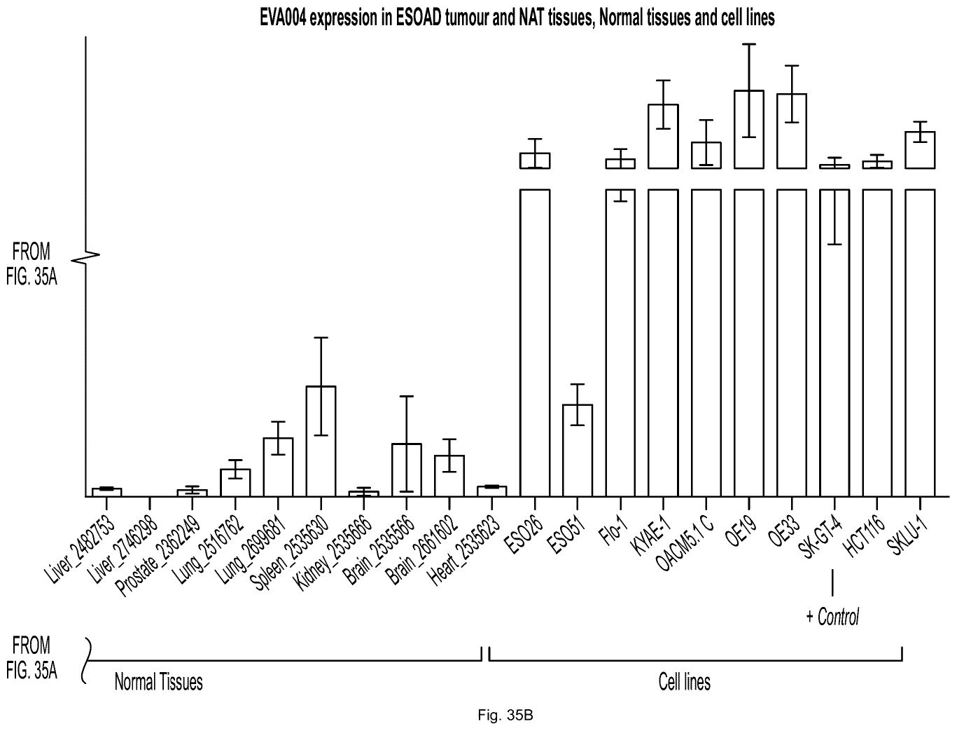

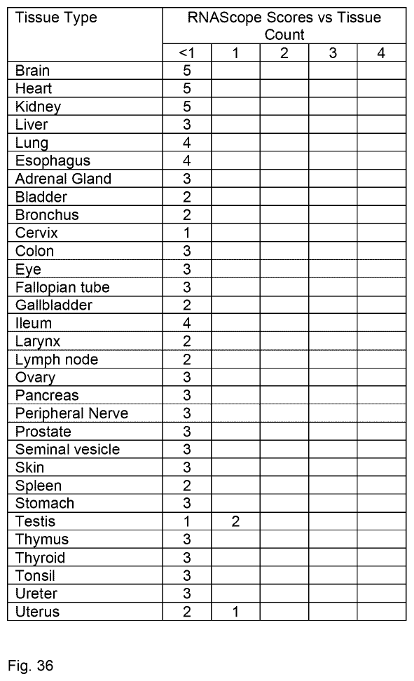

FIG. 36: EVA004 transcript expression in normal tissues by RNAscope probe: EVA004-20zz-RNAscope.

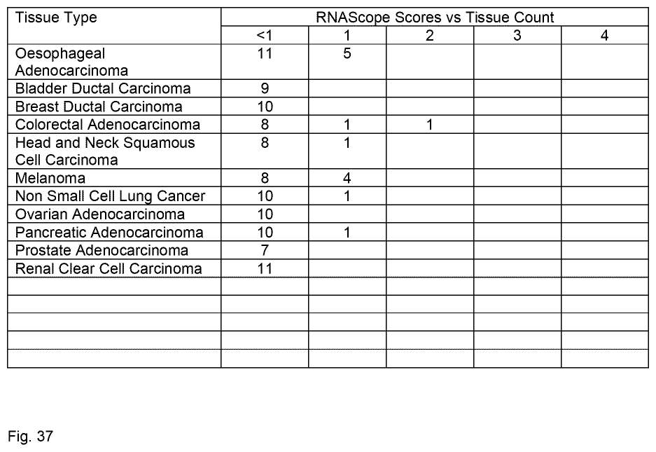

FIG. 37: EVA004 transcript expression in tumour tissues by RNAscope probe: EVA004-20zz-RNAscope.

FIG. 38A-FIG. 38B: EVA007 (SEQ ID NO.7), transcript expression by RT-qPCR screening in oesophageal adenocarcinoma tissues.

FIG. 39A-FIG. 39B: EVA008 (SEQ ID NO.8), transcript expression by RT-qPCR screening in oesophageal adenocarcinoma tissues.

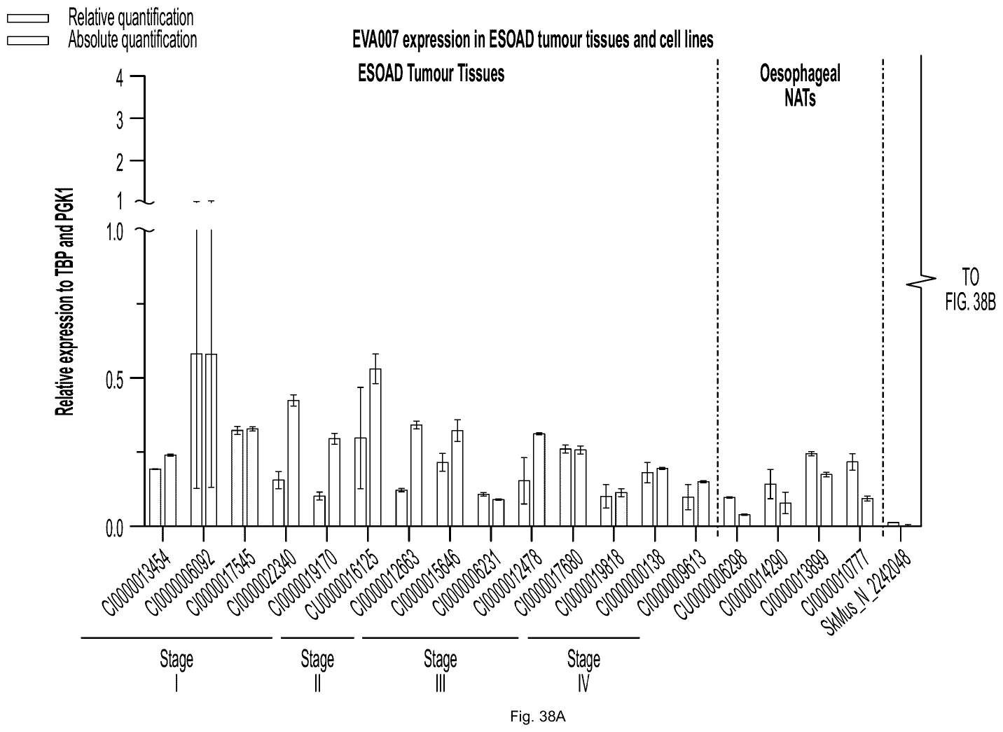

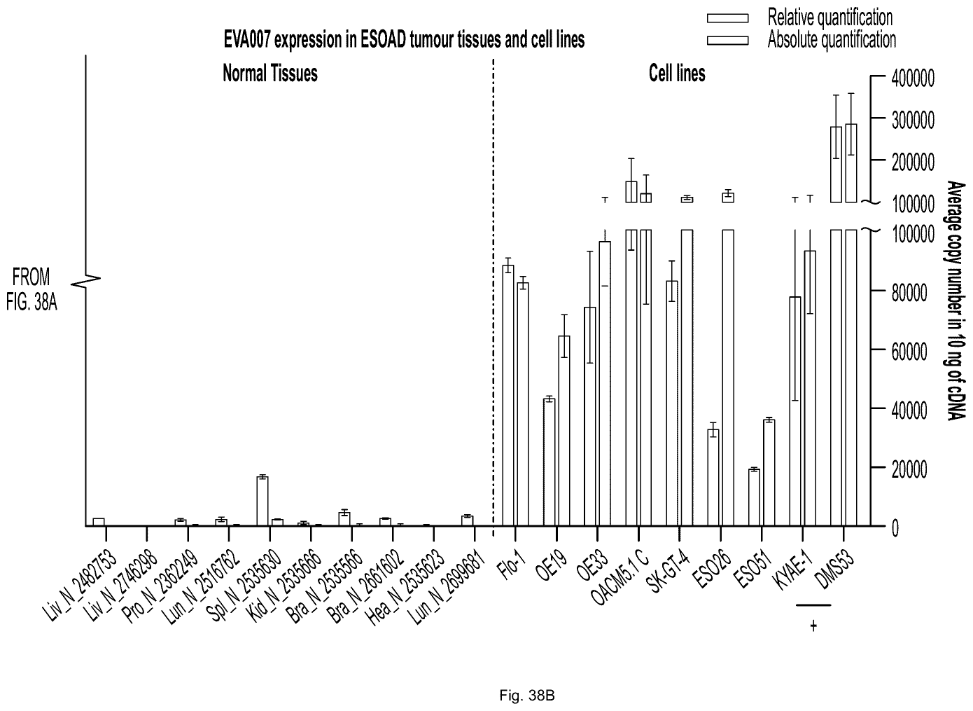

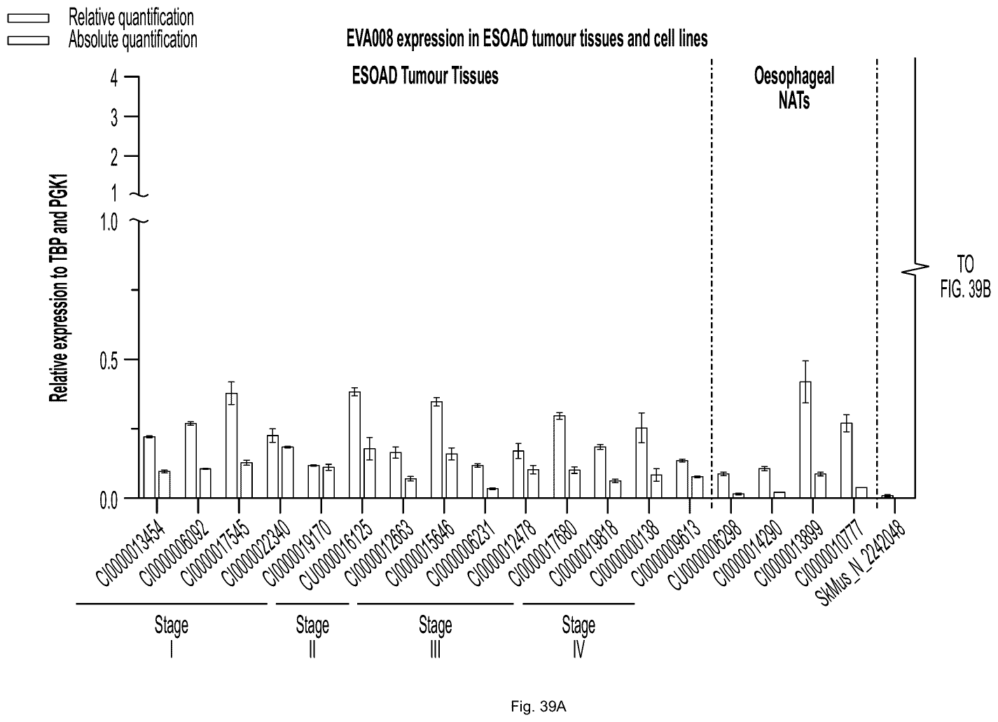

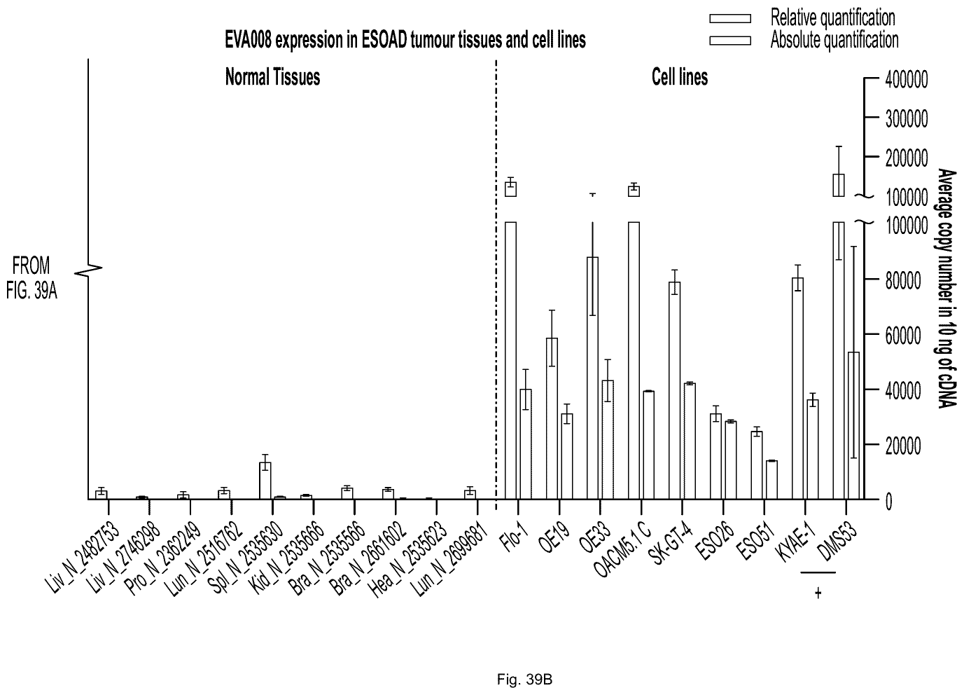

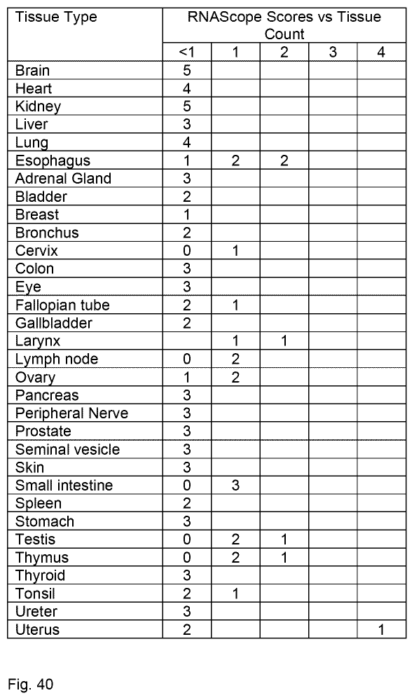

FIG. 40: EVA007/EVA008 transcript expression in normal tissues by RNAscope probe: EVA007-20zz-RNAscope.

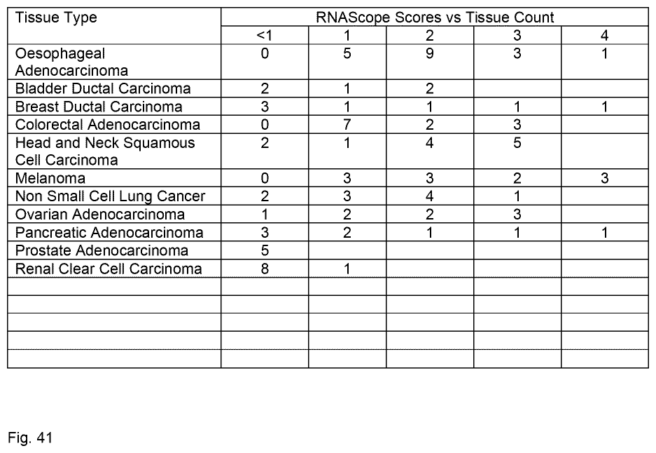

FIG. 41: EVA007/EVA007 transcript expression in tumour tissues by RNAscope probe: EVA007008-20zz-RNAscope.

FIG. 42A-FIG. 42B: EVA005 (SEQ ID NO.5), transcript expression by RT-qPCR screening in oesophageal adenocarcinoma tissues

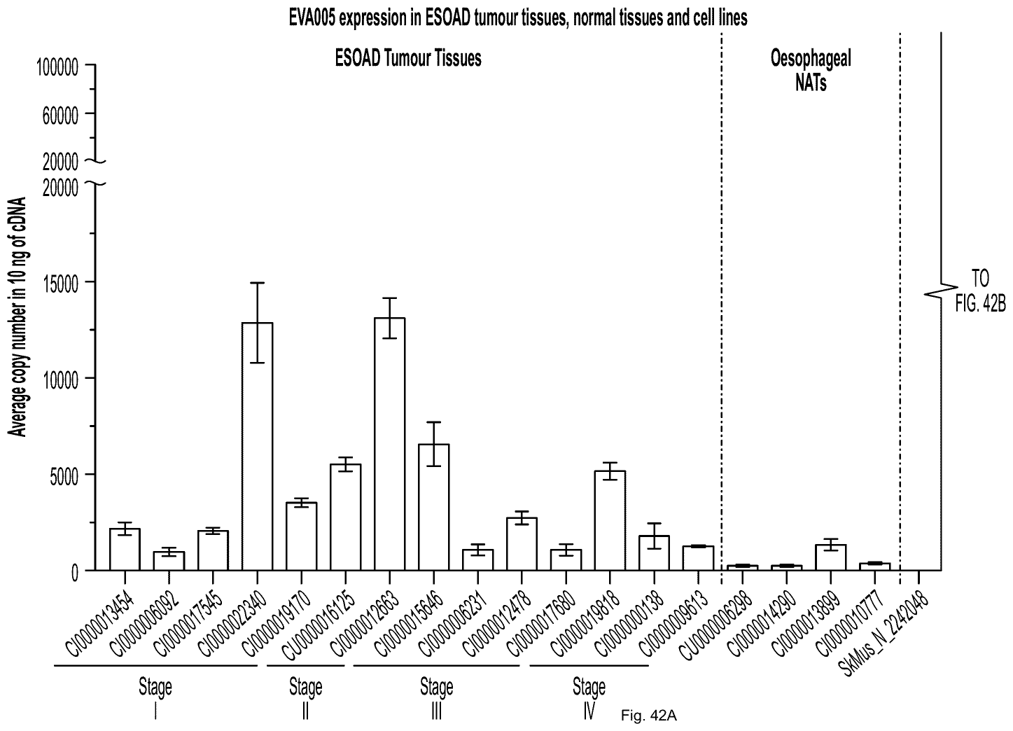

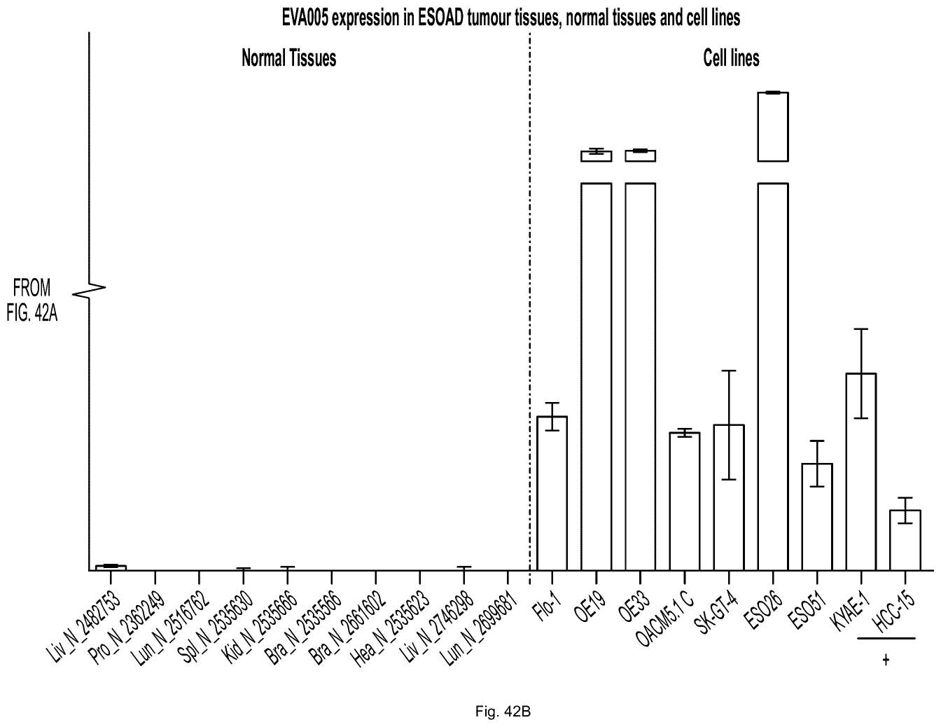

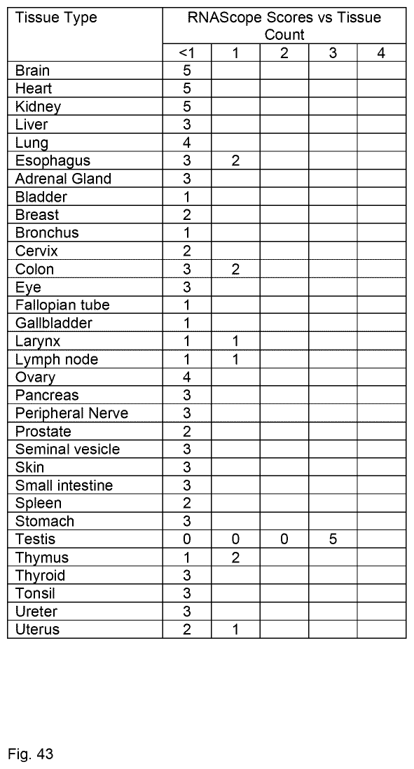

FIG. 43: EVA005 transcript expression in normal tissues by RNAscope probe: EVA005-20zz-RNAscope.

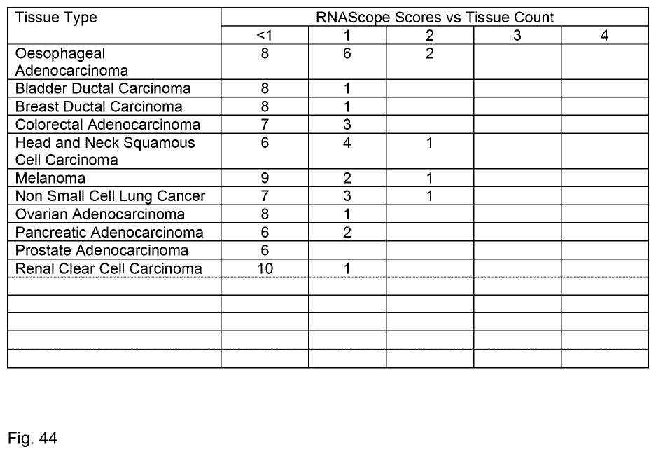

FIG. 44: EVA005 transcript expression in tumour tissues by RNAscope probe: EVA005-20zz-RNAscope.

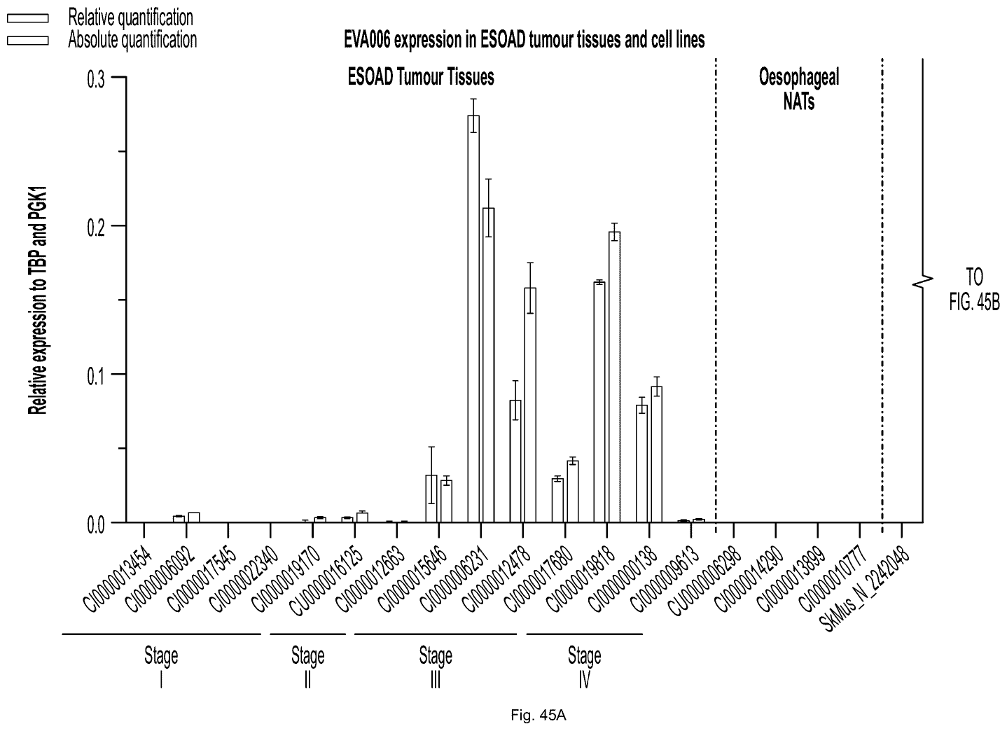

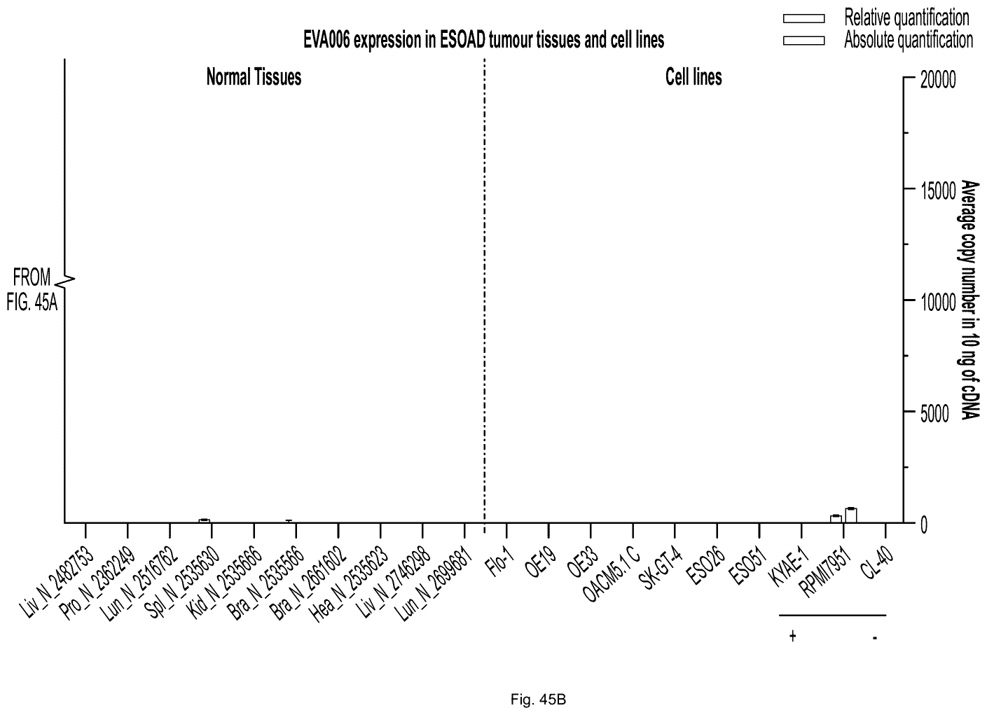

FIG. 45A-FIG. 45B: EVA006 (SEQ ID NO.6), transcript expression by RT-qPCR screening in oesophageal adenocarcinoma tissues

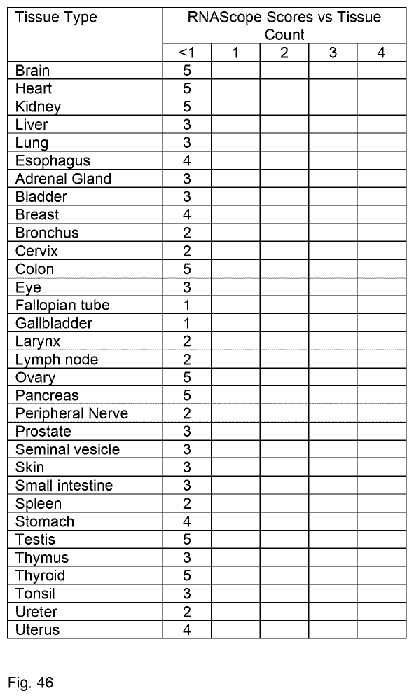

FIG. 46: EVA005 transcript expression in normal tissues by RNAscope probe: EVA006-20zz-RNAscope.

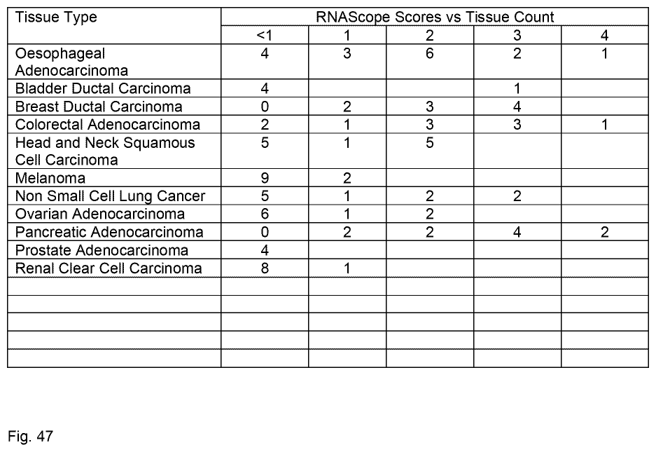

FIG. 47: EVA005 transcript expression in tumour tissues by RNAscope probe: EVA006-20zz-RNAscope.

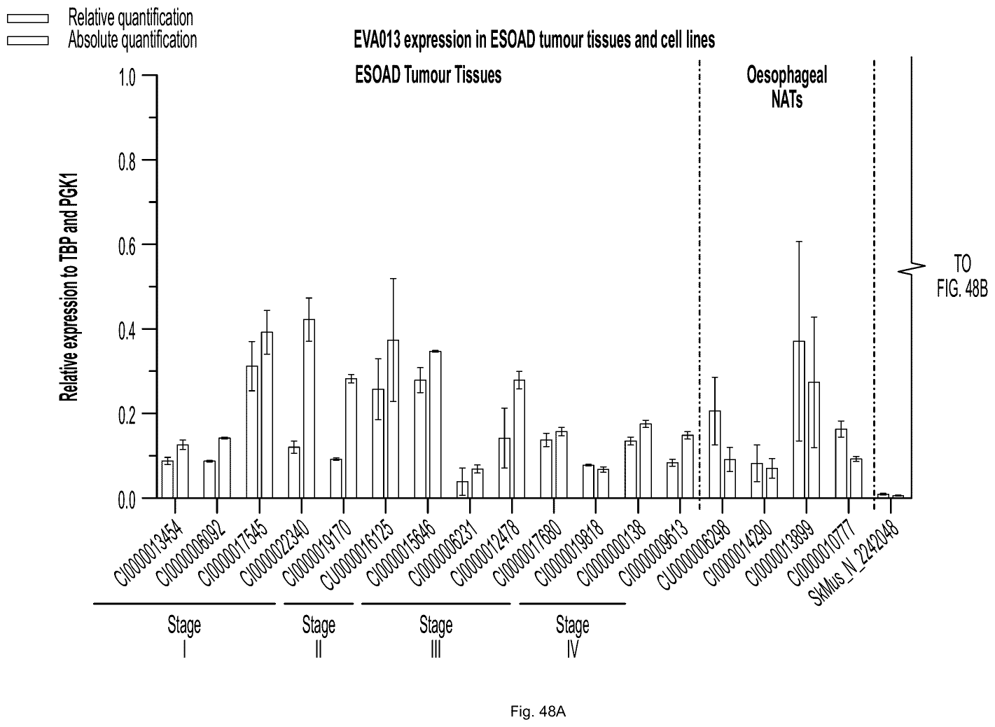

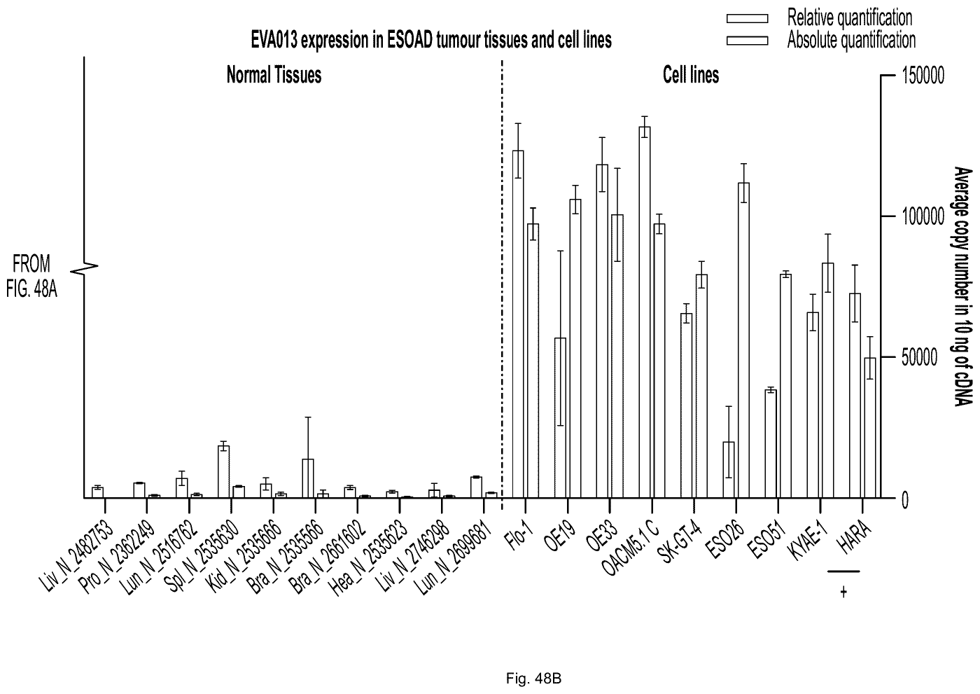

FIG. 48A-FIG. 48B: EVA013 (SEQ ID NO.13), transcript expression by RT-qPCR screening in oesophageal adenocarcinoma tissues

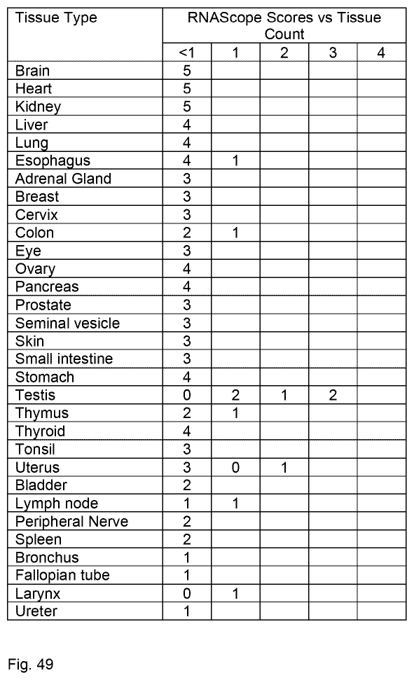

FIG. 49: EVA013 transcript expression in normal tissues by RNAscope probe: EVA013-20zz-RNAscope.

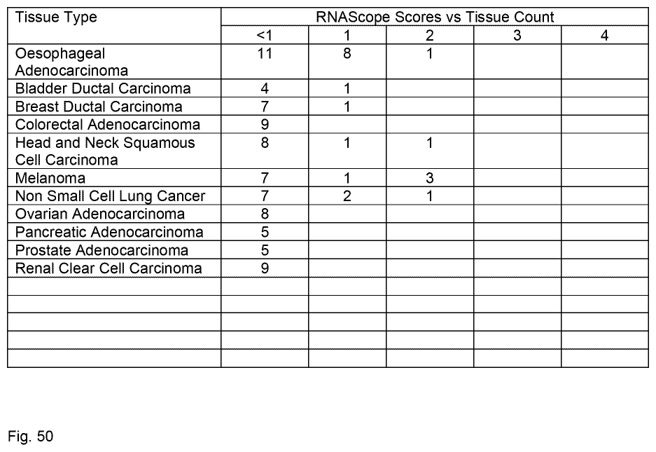

FIG. 50: EVA013 transcript expression in tumour tissues by RNAscope probe: EVA013-20zz-RNAscope.

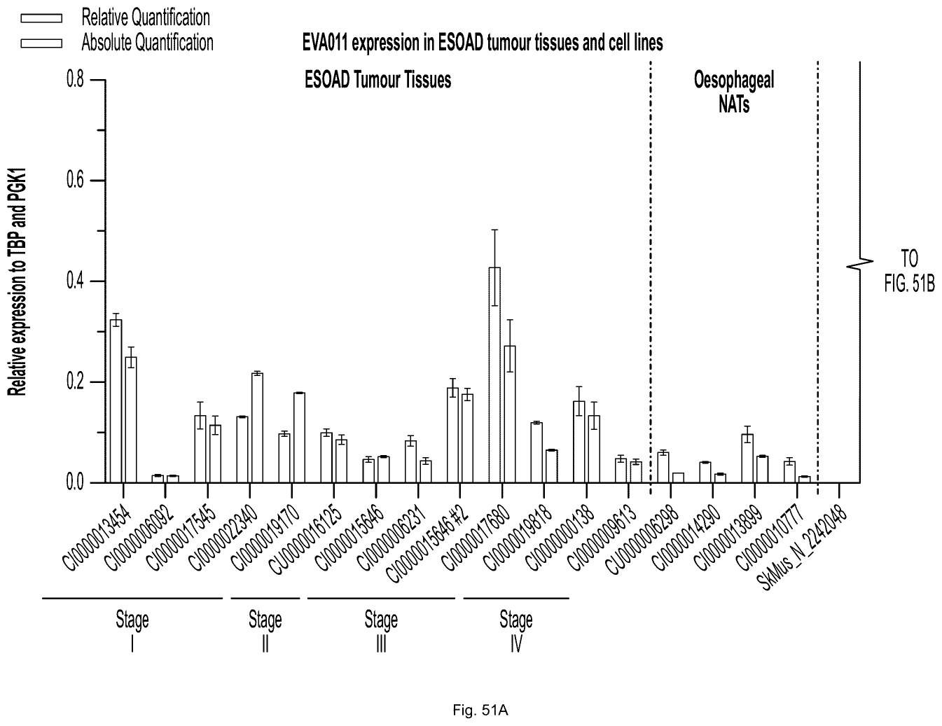

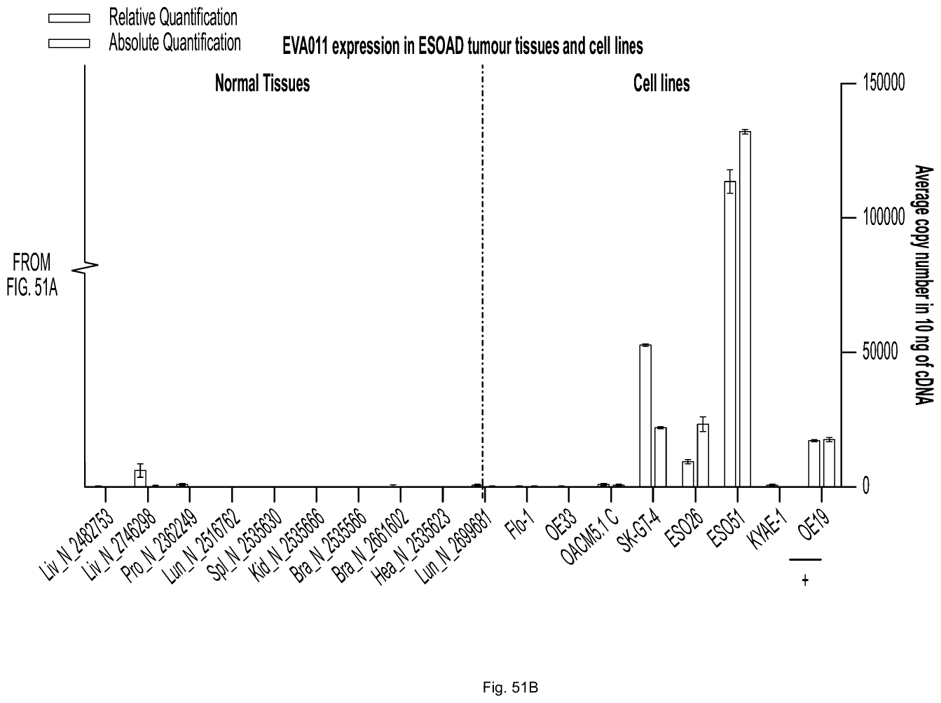

FIG. 51A-FIG. 51B: EVA011 (SEQ ID NO. 11), transcript expression by RT-qPCR screening in oesophageal adenocarcinoma tissues

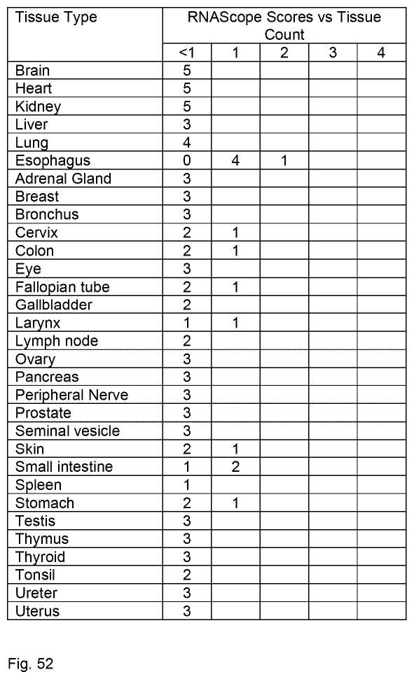

FIG. 52: EVA011 transcript expression in normal tissues by RNAscope probe: EVA011-20zz-RNAscope.

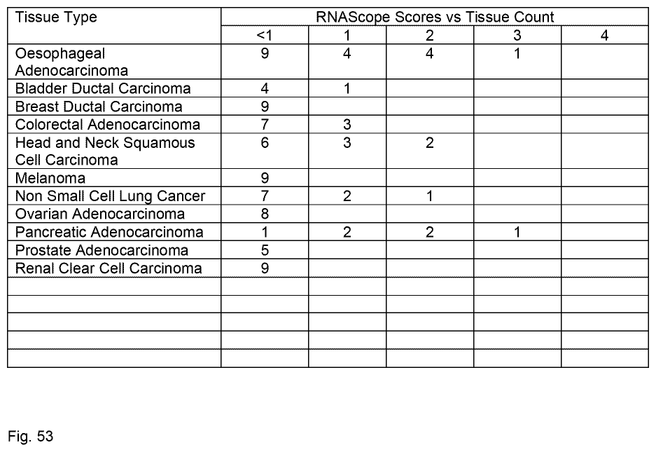

FIG. 53: EVA011 transcript expression in tumour tissues by RNAscope probe: EVA011-20zz-RNAscope.

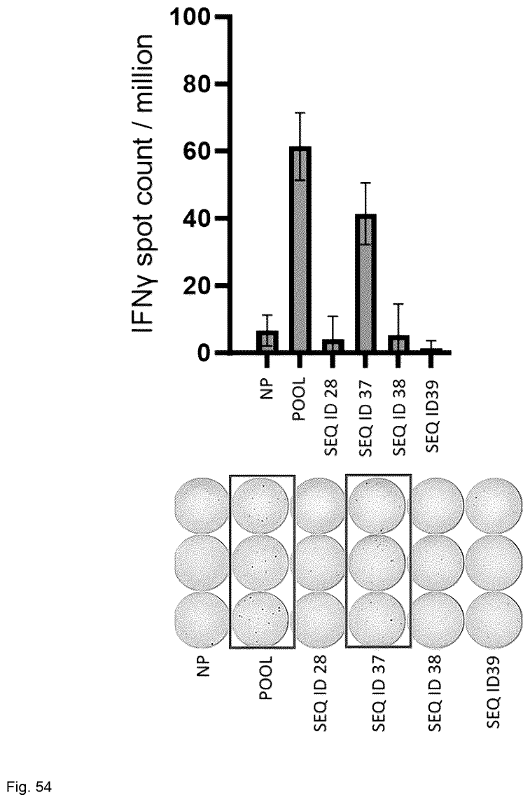

FIG. 54: IFN gamma CD8 T-cell responses from normal blood donors to the HLA restricted CST antigen peptides and bar chart report of IFN gamma CD8 T-cell responses, SEQ ID NO:28 (EVA008), SEQ ID NO:37 (EVA015), SEQ ID NO:38 (EVA015), and SEQ ID NO:39 (EVA015).

FIG. 55: IFN gamma CD8 T-cell responses from normal blood donors to the HLA restricted CST antigen peptides and bar chart report of IFN gamma CD8 T-cell responses, SEQ ID NO:25 (EVA006), SEQ ID NO:24 (EVA005), SEQ ID NO:28 (EVA008), SEQ ID NO:37 (EVA015), SEQ ID NO:38 (EVA015) and SEQ ID NO:39 (EVA015).

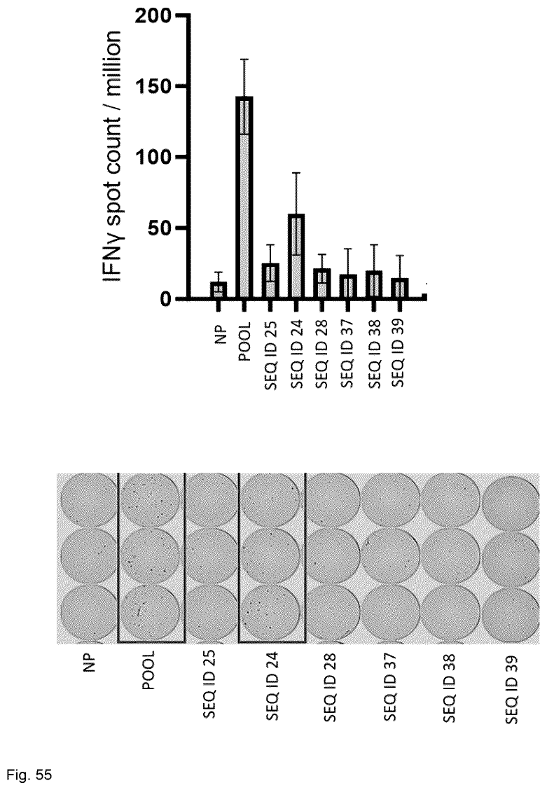

FIG. 56: IFN gamma CD8 T-cell responses from normal blood donors to the HLA restricted CST antigen peptides and bar chart report of IFN gamma CD8 T-cell responses, SEQ ID NO:25 (EVA006), SEQ ID NO:22 (EVA004), SEQ ID NO:24 (EVA005), SEQ ID NO:27 (EVA008), SEQ ID NO:37 (EVA015) and SEQ ID NO:35 (EVA013).

FIG. 57: IFN gamma CD8 T-cell responses from normal blood donors to the HLA restricted CST antigen peptides and bar chart report of IFN gamma CD8 T-cell responses, SEQ ID NO:25 (EVA006), SEQ ID NO:22 (EVA004), SEQ ID NO:24 (EVA005), SEQ ID NO:27 (EVA008), SEQ ID NO:37 (EVA015) and SEQ ID NO:35 (EVA013).

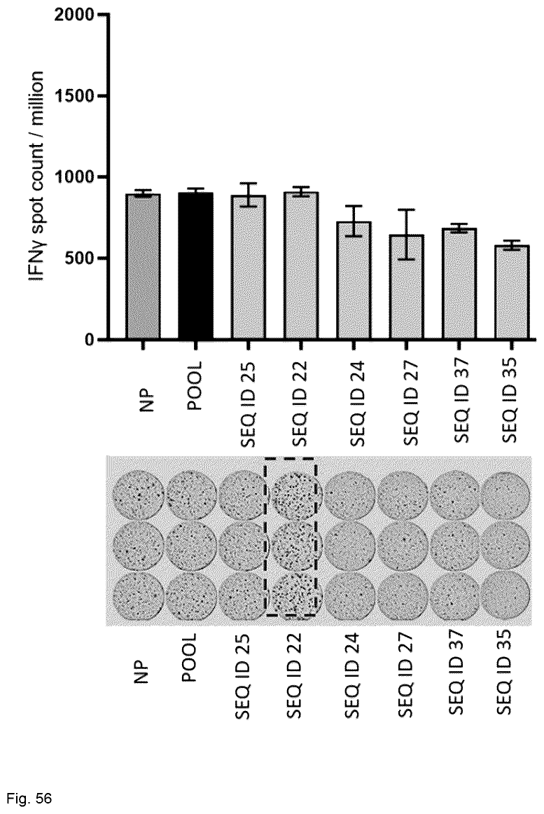

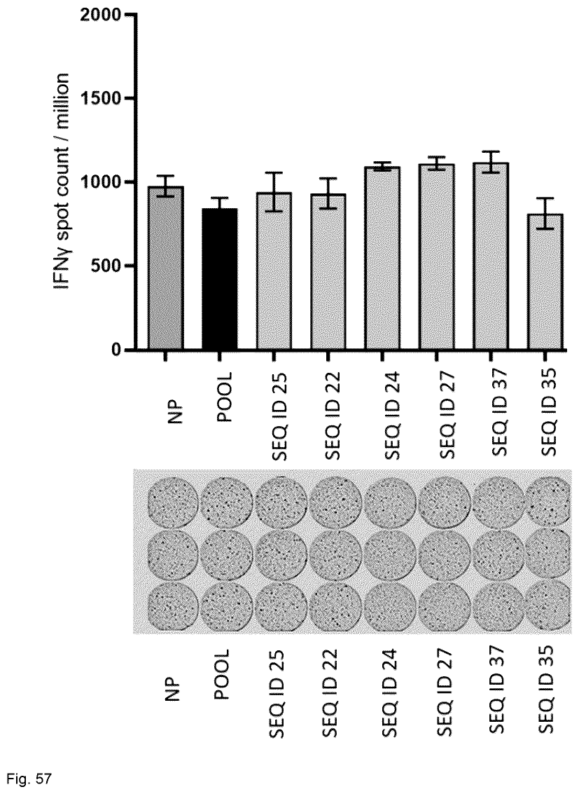

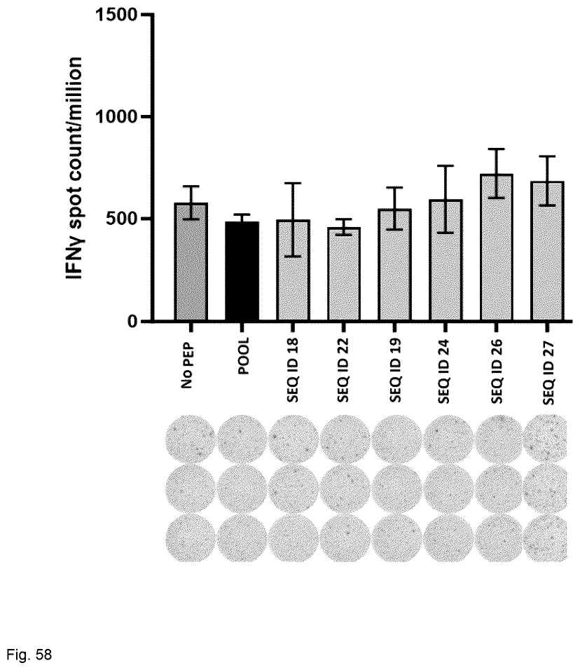

FIG. 58: IFN gamma CD8 T-cell responses from normal blood donors to the HLA restricted CST antigen peptides and bar chart report of IFN gamma CD8 T-cell responses, SEQ ID NO: 18 (EVA001), SEQ ID NO:22 (EVA004), SEQ ID NO: 19 (EVA002), SEQ ID NO:24 (EVA005), SEQ ID NO:26 (EVA007) and SEQ ID NO:27 (EVA008).

### DETAILED DESCRIPTION OF THE INVENTION

### Polypeptides

The terms “protein”, “polypeptide” and “peptide” are used interchangeably herein and refer to any peptide-linked chain of amino acids, regardless of length, co-translational or post-translational modification.

The term “amino acid” refers to any one of the naturally occurring amino acids, as well as amino acid analogs and amino acid mimetics that function in a manner which is similar to the naturally occurring amino acids. Naturally occurring amino acids are those 20 L-amino acids encoded by the genetic code, as well as those amino acids that are later modified, e.g., hydroxyproline, γ-carboxyglutamate, and O-phosphoserine. The term “amino acid analogue” refers to a compound that has the same basic chemical structure as a naturally occurring amino acid, i.e., an a carbon that is bound to a hydrogen, a carboxyl group, an amino group, and an R group but has a modified R group or a modified peptide backbone as compared with a natural amino acid. Examples include homoserine, norleucine, methionine sulfoxide, methionine methyl sulfonium and norleucine. Amino acid mimetics refers to chemical compounds that have a structure that is different from the general chemical structure of an amino acid, but that functions in a manner similar to a naturally occurring amino acid. Suitably an amino acid is a naturally occurring amino acid or an amino acid analogue, especially a naturally occurring amino acid and in particular one of those 20 L-amino acids encoded by the genetic code.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Thus, the invention provides an isolated polypeptide comprising a sequence selected from:
(a) the sequence of any one of SEQ ID NOs. 1 to 17 and 57; and
(b) a variant of the sequence of (a); and
(c) a fragment, such as an immunogenic fragment, of the sequences of (a) and/or (b).

The invention also provides an isolated polypeptide comprising a sequence selected from:
(a) the of any one of SEQ ID NOs. 1 to 17 and 57 minus the initial methionine residue; and
(b) a variant of the sequence of (a); and
(c) a fragment, such as an immunogenic fragment, of the sequences of (a) and/or (b).

In general, variants of polypeptide sequences of the invention include sequences having a high degree of sequence identity thereto. For example, variants suitably have at least about 75% or 80% identity, or at least about any of 81, 82, 83, 84% identity, more preferably at least about 85% identity or at least about any of 86, 87, 88, 89% identity, and most preferably at least about 90% identity (such as any of at least about 91, 92, 93, 94, 96, or 97%, or at least about 98% or at least about 99%) to the associated reference sequence over their whole length.

Suitably the variant (which may be a fragment) is an immunogenic variant. A variant is considered to be an immunogenic variant where it elicits a response which is at least 20%, suitably at least 50% and especially at least 75% (such as at least 90%) of the activity of the reference sequence (i.e. the sequence of which the variant is a variant) e.g., in an in vitro restimulation assay of PBMC or whole blood, or T-cells optionally derived therefrom, with the polypeptide as antigen (e.g., restimulation for a period of between several hours to up to 1 year, such as up to 6 months, 1 day to 1 month or 1 to 2 weeks or for example any of 1, 2, 3, 4, 5, 6, 7, 8, 9, 10 or 11 hours, or 12 to 24 hours, for example any of 14, 16, 18, 20 or 22 hours), that measures the activation of the cells, for example T-cells, via lymphoproliferation (e.g., T-cell proliferation), production of cytokines (e.g., IFN-gamma) in the supernatant of culture (measured by ELISA etc.) or characterisation of T cell responses by intra- and extracellular staining (e.g., using antibodies specific to immune markers, such as CD3, CD4, CD8, IL2, TNF-alpha, IFNg, Type 1 IFN, CD40L, CD69 etc.) followed by analysis with a flow cytometer. For example, the variant can be presented by MHC on an antigen presenting cell like a dendritic cell or on an MHC “multimer” (e.g. dextramer, pentamer e.g. on a solid substrate like a polymer bead or plate).

The polypeptides of the invention, including fragments and variants, are also considered “immunogenic” where they bind to at least one MHC molecule and/or elicit an immune response, for example induce immunoresponsive cells such as T cells for example T-cells cross-reacting with said polypeptide, for example as a complex with said MHC. For example an immunoresponsive cell or T-cell that “cross-reacts” may be characterised by the ability to bind to both the polypeptide and to the polypeptide variant (or polypeptide fragment and polypeptide fragment variant) for example when presented on MHC, e.g. as pMHC, (on MHC multimers or on antigen presenting cells). The elicited “immune response”, for example in an immunoresponsive cell (e.g. T-cell or NK cell) which binds to the polypeptide or pMHC, may be the induction of T-cell proliferation and/or production of cytokines, e.g. IFN-gamma, IL-2, TNF-alpha or for example granzyme and/or immunoresponsive cell induction of tumour cell killing or apoptosis, immune response can be measured in cell proliferation or immunological assays known in the art. The polypeptides of the invention, including fragments and variants, preferably bind to at least one MHC molecule, for example to a MHC I or MHC II molecule, preferably to at least one MHC allele, for example to at least one of an HLA A, B or C allele, preferably to at least one HLA allele selected from the group consisting of HLA-A*01:01, HLA-A*02, HLA-A*02:01, HLA-A*03:01, HLA-A*11:01, HLA-A*24:02, HLA-A*30:01, HLA-A*31:01, HLA-B*07, HLA-B*07:02, HLA-B*08:01, HLA-B*15:01, HLA-B*35:02, HLA-B*37:01, HLA-B*39:01, HLA-B*44:02, HLA-B*51:01, HLA-C*2:02, HLA-C*03:03, HLA-C*03:04, HLA-C*04:01, HLA-C*05:01, HLA-C*06:02, HLA-C*07 and HLA-C*07:02. A variant sequence of a polypeptide preferably maintains the capacity to bind to an MHC molecule or molecules as recited herein, more specifically to the same MHC molecule or molecules and/or induce T cells cross-reacting with said variant.

According to the invention the variant may, for example, be a conservatively modified variant. A “conservatively modified variant” is one where the alteration(s) results in the substitution of an amino acid with a functionally similar amino acid or the substitution/deletion/addition of residues which do not substantially impact the biological function or immunogenicity of the variant, e.g. it will be between 70 to 75% or 75% to 80% or 80 to 90% or 90 to 95%, preferably any of 96, 97, 98, 99% or more the same when tested for example by binding and/or immunological assay. Typically, such immunogenicity or biological function of the variants will be to bind to and/or induce an immune response, for example in an immunoresponsive cell (e.g. T-cell or NK cell) which binds to the polypeptide (or non-variant/unmodified polypeptide) or pMHC thereof, and/or preferably against any one or more of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC or cells, e.g. tumour cells, thereof. immunogenicity or biological function may be tested by assays presented herein, e.g. ELISPOT cytokine assay or (tumour) cell kill assay known in the art and by binding affinity assay known in the art.

Conservative substitution tables providing functionally similar amino acids are well known in the art. Variants can include homologues of polypeptides found in other species.

A variant of a polypeptide of the invention may contain a number of substitutions, for example, conservative substitutions (for example, 1-25, such as 1-10, in particular any of 1, 2, 3, 4 or 5, and especially 1 amino acid residue(s) may be altered) when compared to the reference sequence. The number of substitutions, for example, conservative substitutions, may be up to 20% e.g., up to 10% e.g., up to 5% e.g., up to 1% of the number of residues of the reference sequence. In general, conservative substitutions will fall within one of the amino-acid groupings specified below, though in some circumstances other substitutions may be possible without substantially affecting the immunogenic properties of the polypeptide. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins 1984).

Suitably such substitutions do not alter the immunological structure and/or characteristics of an epitope (e.g., they do not occur within the epitope region as mapped in the primary sequence), and do not therefore have a significant impact on the tertiary conformation or immunogenic properties of the polypeptide.

Polypeptide variants also include those wherein additional amino acids are inserted compared to the reference sequence, for example, such insertions may occur at 1-10 locations (such as any of 1, 2, 3, 4 or 5 locations, suitably 1 or 2 locations, in particular 1 location) and may, for example, involve the addition of 50 or fewer amino acids at each location (such as 20 or fewer, in particular 10 or fewer, especially 5 or fewer for example 4, 3, 2 or 1). Suitably such insertions do not occur in the region of an epitope or within the epitope, and do not therefore have a significant or substantial impact on the immunogenic properties of the polypeptide. One example of insertions includes a short stretch of histidine residues, e.g. at the C or N terminus, (e.g., 2-6 residues) to aid expression and/or purification of the antigen in question.

Polypeptide variants include those wherein amino acids have been deleted compared to the reference sequence, for example, such deletions may occur at 1-10 locations (such as any of 1, 2, 3, 4 or 5 locations, suitably 1 or 2 locations, in particular 1 location) and may, for example, involve the deletion of 50 or fewer amino acids at each location (such as 20 or fewer, in particular 10 or fewer, especially 5 or fewer for example 4, 3, 2, or 1). Suitably such deletions do not occur in the region of an epitope, or within the epitope, and do not therefore have a significant or substantial impact on the immunogenic properties of the polypeptide.

The skilled person will recognise that a particular protein variant may comprise substitutions, deletions and additions (or any combination thereof). For example, substitutions/deletions/additions might enhance (or have neutral effects) on binding to desired patient HLA molecules, potentially increasing immunogenicity (or leaving immunogenicity unchanged).

Fragments, such as immunogenic fragments, or variants thereof, according to the present invention will typically comprise at least 8 or 9 contiguous amino acids from the full-length polypeptide sequence (e.g., at least 7 or 8 to 11, or 9 or 10), such as at least 12 contiguous amino acids (e.g., at least 13 or 14 or 15 or at least 18, for example 16 or 17 or at least 19 or 20 contiguous amino acids), (for example, up to any of 7, 8, 9 10, 11, 12, 13, 14, 15 16, 17, 18, 19 or 20 contiguous amino acids or between any of 8 and 9, 8 and 10, 8 and 11, 8 and 12, 8 and 13, 8 and 14, 8 and 15, 8 and 16, 8 and 17, 8 and 18, 8 and 19, or 8 and 20 contiguous amino acids), alternatively any of 21, 22, 23, 24, 25, 26, 27, 28, 29 or 30 contiguous amino acids, in particular at least 50 contiguous amino acids, such as at least 100 contiguous amino acids (for example at least 200 contiguous amino acids) depending on the length of the CST antigen. Suitably the fragments will be at least 10%, such as at least 20%, such as at least 50%, such as at least 70% or at least 80% of the length of the full-length polypeptide sequence.

Fragments, such as immunogenic fragments, or variants thereof, typically comprise at least one epitope. Epitopes include B cell and T cell epitopes and suitably fragments comprise at least one T-cell epitope such as a CD4+ or a CD8+T-cell epitope.

T cell epitopes are short contiguous stretches of amino acids which are recognised by T cells (e.g., CD4+ or CD8+ T cells) optionally when bound to HLA molecules. Identification of T cell epitopes may be achieved through epitope mapping experiments which are well known to the person skilled in the art (see, for example, Paul, Fundamental Immunology, 3rd ed., 243-247 (1993); Beiβbarth et al., 2005, Bioinformatics, 21(Suppl. 1):i29-i37).

As a result of the crucial involvement of the T cell response in cancer, it is readily apparent that fragments of the full-length polypeptides of SEQ ID NOs. 1 to 17 and 57, or variants thereof, which contain at least one T cell epitope may be immunogenic and may contribute to biological function and/or immunogenicity or immunoprotection (i.e. ability to raise an immune response in an immunoresponsive cell (e.g. T-cell or NK cell) to a cancer or tumour cell disclosed herein, e.g. causing cell death or apoptosis).

It will be understood that in a diverse outbred population, such as humans, different HLA types mean that specific epitopes may not be recognised by all members of the population. Consequently, to maximise the level of recognition and scale of immune response to a polypeptide, it is generally desirable that a fragment, such as an immunogenic fragment, contains a plurality of the epitopes from the full-length sequence (suitably all epitopes within a CST antigen).

Particular fragments of the polypeptides of sequence of any one of SEQ ID NOs. 1 to 17 and 57; or variants thereof which may be of use according to the invention include those containing at least one CD8+ T-cell epitope, suitably at least two CD8+ T-cell epitopes and especially all CD8+ T-cell epitopes, particularly those associated with one or a plurality of HLA Class I alleles, e.g., those associated with 2, 3, 4, 5 or more alleles, for example as hereinabove described). Particular fragments of the polypeptides of SEQ ID NO. 1 to 17 and 57, or variants thereof, which may be of use according to the invention include those containing at least one CD4+ T-cell epitope, suitably at least two CD4+ T-cell epitopes and especially all CD4+ T-cell epitopes (particularly those associated with one or a plurality of HLA Class II alleles, e.g., those associated with 2, 3, 4, 5 or more alleles). However, a person skilled in design of vaccines could combine exogenous CD4+ T-cell epitopes with CD8+ T cells epitopes of this invention and achieve desired responses to the invention's CD8+ T cell epitopes.

Where an individual fragment of the full-length polypeptide is used, such a fragment is considered to be immunogenic where it elicits a response which is at least 20%, suitably at least 50% and especially at least 75%, 80%, 85% (such as at least 90% or 95%) of the activity of the reference sequence (i.e., the sequence of which the fragment is a fragment) e.g., activity in an in vitro restimulation assay of PBMC or whole blood with the polypeptide as antigen (e.g., restimulation for a period of between several hours for example 1, 2, 3, 4, 5, 6, 7, 8, 9 hours or 10 to 24 hours, or 12 to 14 hours or 16 to 18 hours or 20 to 22 hours, to up to 1 year, such as up to 6 months, 1 day to 1 month or 1 to 2 weeks,) that measures the activation of the cells via lymphoproliferation (e.g., NK cell, B-cell or T-cell proliferation), production of cytokines (e.g., IFN-gamma, IL-2, TNF-alpha) in the supernatant of culture (measured by ELISA etc.) or characterisation of immune cell or T cell responses by intra and extracellular staining (e.g., using antibodies specific to immune markers, such as CD3, CD4, CD8, IL2, TNF-alpha, IFN-gamma, Type 1 IFN, CD40L, CD69 etc.) followed by analysis with a flow cytometer.

In some circumstances a plurality of fragments of the full-length polypeptide (which may or may not be overlapping and may or may not cover the entirety of the full-length sequence) may be used to obtain an equivalent biological response to the full-length sequence itself. For example, at least two fragments e.g. immunogenic fragments (such as three, four or five fragments) as described above, which in combination provide at least 50%, suitably at least 75%, 80%, 85% and especially at least 90% to 95% activity of the reference sequence in an in vitro restimulation assay of PBMC or whole blood (e.g., a T cell proliferation and/or IFN-gamma production assay).

Example fragments of polypeptides of SEQ ID NO. 1 to 17 and 57, such as immunogenic fragments thereof, and thus example peptides of the invention, include polypeptides which comprise or consist of the sequence selected from any one of SEQ ID NOs. 18 to 56 and 58, for example, either SEQ ID NOs. 18 to 56 and 58 or variants or immunogenic variants of SEQ ID NOs. 18 to 56 and 58, for example that have 1, 2, 3 or 4, amino acid variations selected from additions, substitutions and deletions with respect thereto. The sequences of SEQ ID NOs. 18 to 56 and 58 were identified as being bound to HLA Class I molecules from immunopeptidomic analysis (see Example 2).

The present invention further relates to the polypeptides according to the present invention, wherein said polypeptide includes non-peptide bonds. In addition, the polypeptide or variant or fragment may be modified further to improve stability and/or binding to MHC molecules to elicit a stronger immune response. Methods for such an optimization of a peptide sequence are well known in the art and include, for example, the introduction of reverse peptide bonds or non-peptide bonds. In a reverse peptide bond, amino acid residues are not joined by peptide (—CO—NH—) linkages but the peptide bond is reversed. Peptides comprising the bonds described above may be synthesized with additional chemical groups present at their amino and/or carboxy termini, to enhance the stability, bioavailability, and/or affinity of the peptides. For example, hydrophobic groups such as carbobenzoxyl, dansyl, or t-butyloxycarbonyl groups may be added to the peptides' amino termini. Likewise, an acetyl group or a 9-fluorenylmethoxy-carbonyl group may be placed at the peptides' amino termini. Additionally, the hydrophobic group, t-butyloxycarbonyl, or an amido group may be added to the peptides' carboxy termini. The present invention further relates to the polypeptides according to the present invention, wherein said polypeptide includes altered steric configuration. For example, the D-isomer of one or more of the amino acid residues of the peptide may be used, rather than the usual L-isomer. For example, at least one of the amino acid residues of the polypeptides or peptides of the invention may be substituted by one of the well-known non-naturally occurring amino acid residues. Alterations such as these may serve to increase the stability, bioavailability and/or binding action of the polypeptides of the invention. Similarly, a polypeptide or fragment or variant of the invention may be modified chemically by reacting specific amino acids either before or after synthesis of the peptide. Chemical modification of amino acids includes, but is not limited to, modification by acylation, amidination, pyridoxylation of lysine, reductive alkylation, trinitrobenzylation of amino groups with 2,4,6-trinitrobenzene sulphonic acid (TNBS), amide modification of carboxyl groups and sulphydryl modification by performic acid oxidation of cysteine to cysteic acid, formation of mercurial derivatives, formation of mixed disulphides with other thiol compounds, reaction with maleimide, carboxymethylation with iodoacetic acid or iodoacetamide and carbamoylation with cyanate at alkaline pH. The present invention further relates to a non-naturally occurring polypeptide wherein said polypeptide consists or consists essentially of an amino acid sequence according to SEQ ID NO: 1 to SEQ ID NO: 58 or fragment or variant, preferably in each case immunogenic, thereof and has been synthetically produced as a pharmaceutically acceptable salt.

### Nucleic Acids

The invention provides an isolated nucleic acid encoding a polypeptide of the invention (referred to as a nucleic acid of the invention). For example, the nucleic acid of the invention comprises or consists of a sequence selected from a sequence encoding any of SEQ ID NOs. 1 to 58 or fragments of variants thereof, preferably immunogenic. Preferably the nucleic acid is an encoding nucleic acid sequence which comprises or consists of a mammalian or human nucleic acid sequence or a sequence according to mammalian, preferably human codon usage and/or codon optimisation. Said sequence may comprise randomly assigned synonymous codons for one or more or each of the encoded amino acids of the polypeptide. Said sequence may include one or more degenerate codons, where the one or more or each codon will encode all possible synonymous codons for a particular amino acid or may comprise preferred codons using most frequent synonymous codon only, or codons chosen with probabilities based on the frequency distribution observed for highly expressed human polypeptide sequences.

The terms “nucleic acid” and “polynucleotide” are used interchangeably herein and refer to a polymeric macromolecule made from nucleotide monomers particularly deoxyribonucleotide or ribonucleotide monomers. The term encompasses nucleic acids containing known nucleotide analogs or modified backbone residues or linkages, which are naturally occurring and non-naturally occurring, which have similar properties as the reference nucleic acid, and which are intended to be metabolized in a manner similar to the reference nucleotides or are intended to have extended half-life in the system. Examples of such analogs include, without limitation, phosphorothioates, phosphoramidates, methyl phosphonates, chiral-methyl phosphonates, 2-O-methyl ribonucleotides, peptide-nucleic acids (PNAs). Suitably the term “nucleic acid” refers to naturally occurring polymers of deoxyribonucleotide or ribonucleotide monomers. Suitably the nucleic acid molecules of the invention are recombinant. Recombinant means that the nucleic acid molecule is the product of at least one of cloning, restriction or ligation steps, or other procedures that result in a nucleic acid molecule that is distinct from a nucleic acid molecule found in nature (e.g., in the case of cDNA). In an embodiment the nucleic acid of the invention is an artificial nucleic acid sequence (e.g., a cDNA sequence or nucleic acid sequence with non-naturally occurring codon usage). In one embodiment, the nucleic acids of the invention are DNA. Alternatively, the nucleic acids of the invention are RNA.

DNA (deoxyribonucleic acid) and RNA (ribounucleic acid) refer to nucleic acid molecules having a backbone of sugar moieties which are deoxyribosyl and ribosyl moieties respectively. The sugar moieties may be linked to bases which are the 4 natural bases (adenine (A), guanine (G), cytosine (C) and thymine (T) in DNA and adenine (A), guanine (G), cytosine (C) and uracil (U) in RNA). As used herein, a “corresponding RNA” is an RNA having the same sequence as a reference DNA but for the substitution of thymine (T) in the DNA with uracil (U) in the RNA. The sugar moieties may also be linked to unnatural bases such as inosine, xanthosine, 7-methylguanosine, dihydrouridine and 5-methylcytidine. Natural phosphodiester linkages between sugar (deoxyribosyl/ribosyl) moieties may optionally be replaced with phosphorothioates linkages. Suitably nucleic acids of the invention consist of the natural bases attached to a deoxyribosyl or ribosyl sugar backbone with phosphodiester linkages between the sugar moieties.

In an embodiment the nucleic acid of the invention is a DNA. For example the nucleic acid comprises or consists of a sequence encoding a sequence selected from any of SEQ ID NOs. 1 to 58. Also provided is a nucleic acid encoding a sequence which comprises or consists of a variant of sequence selected from any of SEQ ID NOs. 1 to 58 which variant encodes the same amino acid sequence but has a different nucleic acid based on the degeneracy of the genetic code.

Thus, due to the degeneracy of the genetic code, a large number of different, but functionally identical nucleic acids can encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations lead to “silent” (sometimes referred to as “degenerate” or “synonymous”) variants, which are one species of conservatively modified variations. Every nucleic acid sequence disclosed herein which encodes a polypeptide also enables every possible silent variation of the nucleic acid. One of skill will recognise that each codon in a nucleic acid (except AUG, which is ordinarily the only codon for methionine, and UGG, which is ordinarily the only codon for tryptophan) can be modified to yield a functionally identical molecule. Accordingly, each silent variation of a nucleic acid that encodes a polypeptide is implicit in each described sequence and is provided as an aspect of the invention.

Degenerate codon substitutions may also be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98).

A nucleic acid of the invention which comprises or consists of a sequence encoding a sequence selected from any of SEQ ID NOs. 1 to 58 or fragment or variant, preferably immunogenic, thereof may contain a number of silent variations (for example, 1-50, such as 1-25, in particular 1-5, such as 1, 2, 3, 4 or 5 and especially 1 codon(s) may be altered) when compared to a reference encoding sequence.

A nucleic acid of the invention may comprise or consist of a sequence encoding a sequence selected from any of SEQ ID NOs. 1 to 58 or fragment or variant, preferably immunogenic, thereof without the initial codon for methionine (i.e. ATG or AUG), or a variant thereof as described above.

In an embodiment the nucleic acid of the invention is an RNA. RNA sequences are provided by the present invention which correspond to a DNA sequence provided and referred to herein and have a ribonucleotide backbone instead of a deoxyribonucleotide backbone and have the sidechain base uracil (U) in place of thymine (T).

Thus a nucleic acid of the invention comprises or consists of the RNA equivalent of a DNA or cDNA sequence which is a nucleic acid comprising or consisting of a sequence encoding a sequence selected from SEQ ID NOs. 1-58 or fragment or variant, preferably immunogenic, wherein the RNA sequence may contain a number of silent variations (for example, 1-50, such as 1-25, in particular 1-5, such as 1, 2, 3, 4 or 5 and especially 1 codon(s) may be altered) when compared to the reference sequence. By “RNA equivalent” is meant an RNA sequence which contains the same genetic information as the reference cDNA or DNA sequence (i.e. contains the same codons with a ribonucleotide backbone instead of a deoxyribonucleotide backbone and having the sidechain base uracil (U) in place of thymine (T)).

The invention also comprises sequences which are complementary to the aforementioned cDNA, DNA and RNA sequences.

In an embodiment, the nucleic acids of the invention are codon optimised for expression in host cell, such as a human host cell.

The nucleic acids of the invention are capable of being transcribed and translated into polypeptides of the invention in the case of DNA nucleic acids, and translated into polypeptides of the invention in the case of RNA nucleic acids. Accordingly the present invention provides an isolated nucleic acid as herein described, encoding a polypeptide according to the invention for example a polypeptide comprising a sequence selected from: (a) the sequence of any of SEQ ID NOs. 1 to 17 and 57; and (b) a variant of the sequence of (a); and (c) a fragment, such as an immunogenic fragment, of the sequences of (a) and (b) for example any one of the sequences of SEQ ID NOs: 18-56 and 58 or fragment or variant, preferably immunogenic, thereof.

### Polypeptides and Nucleic Acids

Suitably, the polypeptides and nucleic acids used in the present invention are isolated. An “isolated” polypeptide or nucleic acid is one that is removed from its original environment. For example, a naturally-occurring polypeptide or nucleic acid is isolated if it is separated from some or all of the coexisting materials in the natural system. A nucleic acid is considered to be isolated if, for example, it is cloned into a vector that is not a part of its natural environment.

“Naturally occurring” when used with reference to a polypeptide or nucleic acid sequence means a sequence found in nature and not synthetically modified.

“Artificial” when used with reference to a polypeptide or nucleic acid sequence means a sequence not found in nature which is, for example, a synthetic modification of a natural sequence, or contains an unnatural sequence.

The term “heterologous” when used with reference to the relationship of one nucleic acid or polypeptide to another nucleic acid or polypeptide indicates that the two or more sequences are not found in the same relationship to each other in nature. A “heterologous” sequence can also mean a sequence which is not isolated from, derived from, or based upon a naturally occurring nucleic acid or polypeptide sequence found in the host organism.

The term “chimeric” when used with respect to a biological molecule such as a polypeptide or nucleic acid means a molecule which is of more than one origin i.e. its sequence comprises a heterologous sequence. The second or further origin may be from within the same organism although in an unnatural association with the first origin or may be from another organism or artificial.

As noted above, polypeptide variants preferably have at least about 65% identity, for example at least about 66, 67, 68 or 69% identity preferably at least about 74% identity, for example at least about 70, 71, 72 or 73% identity, preferably at least about 75% identity, for example at least about 76, 77, 78 or 79% identity, preferably at least about 80% identity, more preferably at least about 85% identity, for example at least about 86, 87, 88 or 89% identity, and most preferably at least about 90% identity (such as at least about 95%, at least about 98% or at least about 99%) to the associated reference sequence over their whole length.

For the purposes of comparing two closely-related polypeptide or polynucleotide sequences, the “% sequence identity” between a first sequence and a second sequence may be calculated. Polypeptide sequences are said to be the same as or identical to other polypeptide sequences, if they share 100% sequence identity over their entire length. Residues in sequences are numbered from left to right, i.e. from N- to C-terminus for polypeptides. The terms “identical” or percentage “identity”, in the context of two or more polypeptide sequences, refer to two or more sequences or sub-sequences that are the same or have a specified percentage of amino acid residues that are the same (i.e., 65% identity, optionally at least 70%, 75%, 80%, 85%, 90%, 95%, 98% or 99% identity over a specified region), when compared and aligned for maximum correspondence over a comparison window. Suitably, the comparison is performed over a window corresponding to the entire length of the reference sequence.

For sequence comparison, one sequence acts as the reference sequence, to which the test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percentage sequence identities for the test sequences relative to the reference sequence, based on the program parameters.

A “comparison window”, as used herein, refers to a segment in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerised implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, 1987, J. Mol. Evol. 35:351-360. The method used is similar to the method described by Higgins & Sharp, 1989, CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., 1984, Nuc. Acids Res. 12:387-395).

Another example of algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. Software for performing BLAST analyses is publicly available through the National Center for Biotechnology Information (website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance.

A “difference” between sequences refers to an insertion, deletion or substitution of a single residue in a position of the second sequence, compared to the first sequence. Two sequences can contain one, two or more such differences. Insertions, deletions or substitutions in a second sequence which is otherwise identical (100% sequence identity) to a first sequence result in reduced % sequence identity. For example, if the identical sequences are 9 residues long, one substitution in the second sequence results in a sequence identity of 88.9%. If the identical sequences are 17 amino acid residues long, two substitutions in the second sequence results in a sequence identity of 88.2%.

Alternatively, for the purposes of comparing a first, reference sequence to a second, comparison sequence, the number of additions, substitutions and/or deletions made to the first sequence to produce the second sequence may be ascertained. An addition is the addition of one residue into the first sequence (including addition at either terminus of the first sequence). A substitution is the substitution of one residue in the first sequence with one different residue. A deletion is the deletion of one residue from the first sequence (including deletion at either terminus of the first sequence).

As used herein, the term “bind”, “specific binding” or “specifically binds” or “specifically binding” in relation to the binding of A to B means that A binds to B, for example at or within a respective specific binding site, domain or pocket, with an affinity typically associated with the binding of ligands to receptors or typically associated with molecules of the immune system, such as antibodies and T-cell receptors, optionally a binding affinity level of micromolar or nanomolar affinity, such that the affinity of binding of A to B exceeds or greatly exceeds that of the binding of A to other molecules not intended to be targeted by A. Binding is selective where the antigen and can be discriminated from unwanted or non-specific interactions. The ability to bind to a specific antigenic determinant can be measured either through an enzyme-linked immunosorbent assay (ELISA) or other techniques familiar to one of skill in the art, e.g. surface plasmon resonance technique and traditional binding assays. Preferably the extent of binding to an unrelated protein is less than about 10% of the binding to the antigen as measured by a suitable technique and preferably with a dissociation constant (K_{D}) of <1 μM, <100 nM, <10 nM, <1 nM, <0.1 nM, <0.01 nM, or <0.001 nM (e.g. 10^{˜8}M or less, e.g. from 10^{˜8}M to 10^{˜13}M, e.g., from 10^{˜9}M to 10^{˜13}M). The term “being specifically bound” is to be interpreted in a similar sense. “Affinity” refers to the magnitude of the sum total of non-covalent interactions between a single binding site of a binding molecule (e.g., a MHC, TCR, antibody) and its binding partner (e.g., a ligand, antigen, antigenic determinant, polypeptide or peptide), hence the “binding affinity” refers to intrinsic binding affinity arising through a 1:1 interaction between members of such a binding pair. Affinity can be measured by methods known in the art, for example Surface Plasmon Resonance (SPR).

### Production of Polypeptides of the Invention

Polypeptides of the invention can be obtained and manipulated using the techniques disclosed for example in Green and Sambrook 2012 Molecular Cloning: A Laboratory Manual 4th Edition Cold Spring Harbour Laboratory Press. In particular, artificial gene synthesis may be used to produce polynucleotides (Nambiar et al., 1984, Science, 223:1299-1301, Sakamar and Khorana, 1988, Nucl. Acids Res., 14:6361-6372, Wells et al., 1985, Gene, 34:315-323 and Grundstrom et al., 1985, Nucl. Acids Res., 13:3305-3316) followed by expression in a suitable organism to produce polypeptides. A nucleic acid or DNA sequence or gene encoding a polypeptide of the invention can be synthetically produced by, for example, solid-phase DNA synthesis. Entire encoding nucleic acid or DNA or gene sequences may be synthesized de novo, without the need for precursor template DNA. To obtain the desired oligonucleotide, the building blocks are sequentially coupled to the growing oligonucleotide chain in the order required by the sequence of the product. Upon the completion of the chain assembly, the product is released from the solid phase to solution, deprotected, and collected. Products can be isolated by high-performance liquid chromatography (HPLC) to obtain the desired oligonucleotides in high purity (Verma and Eckstein, 1998, Annu. Rev. Biochem. 67:99-134). These relatively short segments are readily assembled by using a variety of gene amplification methods (Methods Mol Biol., 2012; 834:93-109) into longer DNA molecules, suitable for use in innumerable recombinant DNA-based expression systems. In the context of this invention one skilled in the art would understand that the polynucleotide sequences encoding the polypeptide antigens described in this invention could be readily used in a variety of vaccine production systems, including, for example, viral vectors.

For the purposes of production of polypeptides of the invention in a microbiological host (e.g., bacterial or fungal), nucleic acids or vectors of the invention will comprise suitable regulatory and control sequences (including promoters, termination signals etc) and sequences to promote polypeptide secretion suitable for protein production in the host. Similarly, polypeptides of the invention could be produced by transducing cultures of eukaryotic cells, mammalian, e.g. human cells (e.g., Chinese hamster ovary cells, HEK-293 cells or drosophila S2 cells) with nucleic acids of the invention which have been combined with suitable regulatory and control sequences (including promoters, termination signals etc) and sequences to promote polypeptide secretion suitable for protein production in these cells. Hence the present invention provides a nucleic acid of the invention, or one or more nucleic acids of the invention, further comprising a regulatory and/or control sequence suitable for the production of the encoded polypeptide of the invention.

Improved isolation of the polypeptides of the invention produced by recombinant means may optionally be facilitated through the addition of a stretch of histidine residues (commonly known as a His-tag) towards one end of the polypeptide. Polypeptides may also be produced synthetically.

### Vectors

In additional embodiments, genetic constructs comprising one or more of the nucleic acids of the invention are introduced into cells in vivo such that a polypeptide of the invention, or one or more thereof, is produced, optionally as a fusion protein or expressed separately and/or simultaneously, in vivo eliciting an immune response. The nucleic acid (e.g., DNA) may be present within any of a variety of delivery systems known to those of ordinary skill in the art, including nucleic acid expression systems, bacteria and some viral expression systems. Numerous gene delivery techniques are well known in the art, such as those described by Rolland, 1998, Crit. Rev. Therap. Drug Carrier Systems 15:143-198, and references cited therein. Several of these approaches are outlined below for the purpose of illustration.

Accordingly, there is provided a vector (also referred to herein as a ‘DNA expression construct’ or ‘construct’) comprising a nucleic acid molecule of the invention.

Suitably, the vector comprises regulatory elements (such as a suitable promoter and terminating signal) suitable for permitting transcription of a translationally active RNA molecule in a host cell such as a human host cell. A “translationally active RNA molecule” is an RNA molecule capable of being translated into a protein by a human cell's translation apparatus.

Accordingly, there is provided a vector comprising a nucleic acid of the invention (herein after a “vector of the invention”).

In particular, the vector may be a viral vector. The viral vector may be an adenovirus, adeno-associated virus (AAV) (e.g., AAV type 5 and type 2), alphavirus (e.g., Venezuelan equine encephalitis virus (VEEV), Sindbis virus (SIN), Semliki Forest virus (SFV)), herpes virus, arenavirus (e.g., lymphocytic choriomeningitis virus (LCMV)), measles virus, poxvirus (such as modified vaccinia Ankara (MVA)), paramyxovirus, lentivirus, or rhabdovirus (such as vesicular stomatitis virus (VSV)) vector i.e. the vector may be derived from any of the aforementioned viruses. Adenoviruses are particularly suitable for use as a gene transfer vector because of its mid-sized genome, ease of manipulation, high titre, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP is particularly efficient during the late phase of infection, and all the mRNAs transcribed from this promoter possess a 5′-tripartite leader (TPL) sequence which makes them preferred mRNAs for translation. Replication-deficient adenovirus, which are created by from viral genomes that are deleted for one or more of the early genes are particularly useful, since they have limited replication and less possibility of pathogenic spread within a vaccinated host and to contacts of the vaccinated host.

### Other Polynucleotide Delivery

In certain embodiments of the invention, the expression construct comprising one or more polynucleotide sequences may simply consist of naked recombinant DNA plasmids. Hence the present invention provides a DNA plasmid comprising a nucleic acid of the invention, or one or more nucleic acids of the invention, further comprising a regulatory and/or control sequence suitable for the expression or production of the encoded polypeptide or polypeptides of the invention. See Ulmer et al., 1993, Science 259:1745-1749 and reviewed by Cohen, 1993, Science 259:1691-1692. Transfer of the construct may be performed, for example, by any method which physically or chemically permeabilises the cell membrane. This is particularly applicable for transfer in vitro but it may be applied to in vivo use as well. It is envisioned that DNA encoding a gene of interest may also be transferred in a similar manner in vivo and express the gene product. Multiple delivery systems have been used to deliver DNA molecules into animal models and into man. Some products based on this technology have been licensed for use in animals, and others are in phase 2 and 3 clinical trials in man.

### RNA Delivery

In other embodiments of the invention, the expression construct comprising one or more polynucleotide sequences may consist of naked, recombinant DNA-derived RNA molecules (Ulmer et al., 2012, Vaccine 30:4414-4418). Hence the present invention provides a DNA derived RNA molecule or plasmid comprising a nucleic acid of the invention, or one or more nucleic acids of the invention, optionally further comprising a regulatory and/or control sequence suitable for the expression or production of the encoded polypeptide of the invention. As for DNA-based expression constructs, a variety of methods can be utilized to introduce RNA molecules into cells in vitro or in vivo. The RNA-based constructs can be designed to mimic simple messenger RNA (mRNA) molecules, such that the introduced biological molecule is directly translated by the host cell's translation machinery to produce its encoded polypeptide in the cells to which it has been introduced. Alternatively, RNA molecules may be designed in a manner that allows them to self-amplify within cells they are introduced into, by incorporating into their structure genes for viral RNA-dependent RNA polymerases. Thus, these types of RNA molecules, known as self-amplifying mRNA (SAM™) molecules (Geall et al. 2012, PNAS, 109:14604-14609), share properties with some RNA-based viral vectors. Either mRNA-based or SAM™ RNAs may be further modified (e.g., by alteration of their sequences, or by use of modified nucleotides) to enhance stability and translation (Schlake et al., RNA Biology, 9:1319-1330), and both types of RNAs may be formulated (e.g., in emulsions (Brito et al., Molecular Therapy, 2014 22:2118-2129) or lipid nanoparticles (Kranz et al., 2006, Nature, 534:396-401)) to facilitate stability and/or entry into cells in vitro or in vivo. Myriad formulations of modified (and non-modified) RNAs have been tested as vaccines in animal models and in man, and multiple RNA-based vaccines are being used in ongoing clinical trials.

### Pharmaceutical Compositions

The polypeptides, nucleic acids and vectors of the invention may be formulated for delivery in pharmaceutical compositions such as immunogenic compositions and vaccine compositions (all hereinafter “compositions of the invention”). Compositions of the invention suitably comprise a polypeptide, nucleic acid or vector of the invention together with a pharmaceutically acceptable carrier.

Thus, in an embodiment, there is provided a pharmaceutical composition such as an immunogenic pharmaceutical composition comprising a polypeptide, nucleic acid or vector of the invention together with a pharmaceutically acceptable carrier or pharmaceutically acceptable salt.

In another embodiment there is provided a vaccine composition comprising a polypeptide, nucleic acid or vector of the invention together with a pharmaceutically acceptable carrier and or adjuvant. Preparation of pharmaceutical compositions is generally described in, for example, Powell & Newman, eds., Vaccine Design (the subunit and adjuvant approach), 1995. Compositions of the invention may also contain other compounds, which may be biologically active or inactive. Suitably, the composition of the invention is a sterile composition suitable for parenteral administration. It will be understood that vaccine compositions include therapeutic vaccine compositions as well as prophylactic vaccine compositions.

In certain preferred embodiments of the present invention, pharmaceutical compositions of the invention are provided which comprise one or more (e.g., one) polypeptides of the invention in combination with a pharmaceutically acceptable carrier or pharmaceutically acceptable salt.

In certain preferred embodiments of the present invention, compositions of the invention are provided which comprise one or more (e.g., one) nucleic acids of the invention or one or more (e.g., one) vectors of the invention in combination with a pharmaceutically acceptable carrier or pharmaceutically acceptable salt.

In an embodiment, the compositions of the invention may comprise one or more (e.g., one) polynucleotide of the invention and/or one or more (e.g., one) polypeptide of the invention as components. Alternatively, the compositions may comprise one or more (e.g., one) vector and/or one or more (e.g., one) polypeptide components. Alternatively, the compositions may comprise one or more (e.g., one) vector and/or one or more (e.g., one) polynucleotide of the invention as components. Such compositions may provide for an enhanced immune response.

### Pharmaceutically Acceptable Salts

It will be apparent that a composition of the invention may contain pharmaceutically acceptable salts of the nucleic acids, vectors or polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts). For example, the pharmaceutically acceptable salt may comprise an anion selected from any of chloride, sulfate, maleate; chloride, and acetate and/or cation selected from sodium, potassium, magnesium and calcium.

### Pharmaceutically Acceptable Carriers

While many pharmaceutically acceptable carriers known to those of ordinary skill in the art may be employed in the compositions of the invention, the optimal type of carrier used will vary depending on the mode of administration. Compositions of the present invention may be formulated for any appropriate manner of administration, including for example, parenteral, topical, oral, nasal, intravenous, intracranial, intraperitoneal, subcutaneous or intramuscular administration, preferably parenteral e.g., intramuscular, subcutaneous or intravenous administration. For parenteral administration, the carrier preferably comprises water and may contain buffers for pH control, stabilising agents e.g., surfactants and amino acids and tonicity modifying agents e.g., salts and sugars. If the composition is intended to be provided in lyophilised form for dilution at the point of use, the formulation may contain a lyoprotectant e.g., sugars such as trehalose. For oral administration, any of the above carriers or a solid carrier, such as mannitol, lactose, starch, magnesium stearate, sodium saccharine, talcum, cellulose, glucose, sucrose, and magnesium carbonate, may be employed.

Thus, compositions of the invention may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, solutes that render the formulation isotonic, hypotonic or weakly hypertonic with the blood of a recipient, suspending agents, thickening agents and/or preservatives. Alternatively, compositions of the invention may be formulated as a lyophilizate.

### Immunostimulants

Compositions of the invention may also comprise one or more immunostimulants. An immunostimulant may be any substance that enhances or potentiates an immune response (antibody and/or cell-mediated) to an exogenous antigen. Examples of immunostimulants, which are often referred to as adjuvants in the context of vaccine formulations, include aluminium salts such as aluminium hydroxide gel (alum) or aluminium phosphate, saponins including QS21, immunostimulatory oligonucleotides such as CPG, oil-in-water emulsion (e.g., where the oil is squalene), aminoalkyl glucosaminide 4-phosphates, lipopolysaccharide or a derivative thereof e.g., 3-de-O-acylated monophosphoryl lipid A (3D-MPL) and other TLR4 ligands, TLR7 ligands, TLR8 ligands, TLR9 ligands, IL-12 and interferons. Further suitable examples include: Incomplete Freund's Adjuvant (IFA), aluminum-based adjuvants, e.g. Aluminum hydroxide (Alhydrogel™), Aluminum phosphate (Adjut-phos™), Oil-in-Water emulsions, e.g. MF59, AS03; immunopotentiators, e.g. TLR agonist-based adjuvants, e.g. TLR2 Agonists, e.g. bacterial lipopeptides (e.g., MALP-2, Pam2Cys, Pam3Cys); TLR3 agonists, e.g. palmitic acid-modified TLR7/8 agonist (C16-R848), polyinosine-polycytidylic acid (poly I: C), poly ICLC (Hiltonol), poly I: C12U; TLR4 agonists, e.g. monophosphoryl lipid A (MPLA); TLR7/8 agonists, e.g. Imiquimod (Aldara cream), Resiquimod; TLR9 agonists, e.g. CpG Oligonucleotides (ODN), for example CpG A, CpG B, CpG C, CpG ODN 7909, IC31; Synthetic double-stranded RNAs (dsRNAs), e.g. Poly-I:C, Poly-ICLC; Glucopyranosyl lipid A (GLA), e.g. GLA-SE, GLA-AF, GLA-LS, GLA-Alum; Imidazoquinolines, e.g. Imiquimod (R837), Resiquimod (R848), 3M-052; Cyclic Dinucleotides (CDNs), e.g. 2′,3′-cGAMP, 3′,3′-cGAMP, c-di-GMP, c-di-AMP; Manganese-based adjuvants, e.g. Mn2+ solutions, Manganese Jelly (MnJ), NanoMn; Metabolic adjuvants, e.g. bisphosphonates, statins; Nanoparticle-based adjuvant, e.g. lipid nanoparticles (LNPs), poly (lactide-co-glycolide) (PLGA) particles, chitosan; Water-in-oil nanoemulsions, e.g. Montanide ISA 51, Montanide ISA 720; Micro or nanoparticle adjuvants, e.g. Poly (lactic-co-glycolic acid) (PLGA) particles, Poly (lactic acid) (PLA), Polystyrene particles, Liposomes (e.g., coated with polyethylene glycol (PEG) or modified as immunoliposomes), Poly (DL-lactide-co-glycolide) microspheres, Poly (propylene sulfide) nanoparticles, cGAS-STING/STING agonists, e.g. Cyclic GMP-AMP (cGAMP) and its analogs, CpG ODN 1018, AS04 (contains Monophosphoryl Lipid A), STING agonists like CF501 and ADU-S100; Cytokines, for example, Interleukin-2 (IL-2), e.g. IL-2 muteins, IL-2 pre-complexed with antibodies, IL-2 conjugated with polyethylene glycol, Granulocyte-Macrophage Colony-Stimulating Factor (GM-CSF), Interferons, e.g. IFN-α, IFN-β, IFN-γ; Self-assembling peptides, e.g. nanofiber-forming peptides for adjuvant-free vaccines, Virus-like particles (VLPs), e.g. Qβ, cowpea mosaic virus (CPMV); Inorganic nanoparticles, e.g. gold nanoparticles (AuNPs), silica nanoparticles, mesoporous silica rods (MSRs), calcium phosphate nanoparticles, iron oxide nanoparticles, manganese-based nanoparticles, NanoMn, MnJ (Manganese Jelly), carbon-based nanomaterials, graphene oxide and carbon nanotubes; Caged protein nanoparticles, e.g. ferritin nanoparticles, virus-like particles (VLPs), self-assembling protein nanoparticles (SAPNs), heat shock protein-nanoparticles (HSPs). Additonal suitable examples, particularly useful in nucleic acid antigen vaccines and compositions include: Nucleoside-unmodified mRNAs, for example Poly-uracil (U) sequences, double-stranded RNAs (dsRNA), RNA molecules without nucleoside modifications, RNA oligonucleotides with phosphorothioate internucleotide linkages, short double-stranded RNA (dsRNA) with 5′-triphosphate ends; PRR Ligands (pattern recognition receptor ligands), e.g. IRFs, interferon regulatory factors such as NF-κB, nuclear factor kappa light chain; reactive oxygen species (ROS) such as NLRP3, NOD, LRR, and pyrin domain-containing protein like 3; 5′-ppp, 5′ triphosphate; mRNA encoding functional proteins, e.g. STING, a combination of CD40L, CD70, and a constitutively active TLR4; Component lipid-based adjuvants, e.g. ionizable cationic lipids in LNPs, e.g. DLinDMA, C12-TLRa, lipid components (DOTMA and DOPE), A2-Iso5-2DC18 (A2-Ionizable cationic lipid-like material), SAL12 (C12-TLRaLipidoid), A2 (C1 Cationic lipid-like material), ionizable amino lipid, LCP Lipid-coated calcium phosphate, DOPA dioleoylphosphatydic acid, DLinDMA 1,2-dilinoleyloxy-n,n-dimethyl-3-aminopropane, DDA Dimethyldioctadecylammonium, DOTMA trimethyl[2,3-(dioleyloxy) propyl] ammonium Chloride, DOPE 1,2-Dioleoyl-sn-glycero-3-phosphoethanolamine; Peptide and glycolipid immunostimulants, e.g. lipid ALC-0315, ionizable lipid SM-102, protamine, DP7 (cationic peptide), a-GC glycolipid, arginine-rich protamine peptides, cholesterol-modified cationic peptide DP7, liposomes with DP7, palmitic acid-modified TLR7/8 agonist R848 (C16-R848). Thus, suitably the one or more immunostimulants of the composition of the invention are selected from aluminium salts, saponins, immunostimulatory oligonucleotides, oil-in-water emulsions, aminoalkyl glucosaminide 4-phosphates, lipopolysaccharides and derivatives thereof and other TLR4 ligands, TLR7 ligands, TLR8 ligands and TLR9 ligands, IL12, interferons, incomplete freund's adjuvant (IFA), aluminum-based adjuvants, TLR agonists, synthetic double-stranded RNAs (dsRNAs), glucopyranosyl lipid a (GLA), imidazoquinolines, CPG oligodeoxynucleotides (ODNs), cyclic dinucleotides (CDNs), manganese-based adjuvants; metabolic adjuvants, nanoparticle-based adjuvant, water-in-oil nanoemulsions, micro or nanoparticle adjuvants, CGAS-STING/STING agonists, cytokines, self-assembling peptides, virus-like particles (VLPs), inorganic nanoparticles, caged protein nanoparticles, nucleoside-unmodified mRNA, PRR ligands (pattern recognition receptor ligands), mRNA encoding functional proteins, component lipid-based adjuvants, peptide and glycolipid immunostimulants, TLR agonists. Additionally or alternatively the one or more immunostimulants of the composition of the invention may also be selected from monoclonal antibodies which specifically interact with other immune components, for example monoclonal antibodies that block the interaction of immune checkpoint receptors, including PD-1 and CTLA4.

In the case of recombinant-nucleic acid methods of delivery (e.g., DNA, RNA, viral vectors), the genes encoding protein-based immunostimulants may be readily delivered along with the genes encoding the polypeptides of the invention.

### Sustained Release

The compositions described herein may be administered as part of a sustained-release formulation (i.e., a formulation such as a capsule, sponge, patch or gel (composed of polysaccharides, for example)) that effects a slow/sustained release of compound following administration.

### Storage and Packaging

Compositions of the invention may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a composition of the invention may be stored in a freeze-dried condition requiring only the addition of a sterile liquid carrier (such as water or saline for injection) immediately prior to use.

### Dosage

The amount of nucleic acid, polypeptide or vector in each composition of the invention may be prepared is such a way that a suitable dosage for therapeutic or prophylactic use will be obtained. Factors such as solubility, bioavailability, biological half-life, route of administration, product shelf life, as well as other pharmacological considerations will be contemplated by one skilled in the art of preparing such compositions, and as such, a variety of dosages and treatment regimens may be desirable.

Typically, compositions comprising a therapeutically or prophylactically effective amount deliver about 0.1 ug to about 1000 ug of polypeptide of the invention per administration, more typically about 2.5 ug to about 100 ug of polypeptide per administration. If delivered in the form of short, synthetic long peptides, doses could range from 1 to 200 ug/peptide/dose. In respect of polynucleotide compositions, these typically deliver about 10 ug to about 20 mg of the nucleic acid of the invention per administration, more typically about 0.1 mg to about 10 mg of the nucleic acid of the invention per administration.

### Diseases to be Treated or Prevented

As noted elsewhere, SEQ ID NOs. 1 to 17 and 57 and SEQ ID Nos. 18 to 56 and 58 are polypeptide sequences corresponding to CST antigens which are over-expressed in ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC (see Table 1).

TABLE 1
Correspondence between CST antigen and cancer indication
as determined by transcript expression using Mass Spectrometry
and RNA-Scope analysis (see Examples 2 and 3 herein)

| | | Cancer indication |
|---|---|---|
| | Cancer types showing presence | determined by transcript |
| | of Mass Spectrometry detected | expression in tumour |
| CST | and inferred transcripts of CST | tissue by RNA-Scope |
| Antigen | antigens (Example 1). | analysis (Example 4) |
| EVA001, | ESCA, HNSC, UCSC | Oesophageal, head and |
| (SEQ ID | | neck, melanoma, |
| NO. 1) | | bladder, lung, ovarian |
| EVA002 | ESCA, DLBC, PRAAD, | |
| (SEQ ID | COAD. | |
| NO. 2) | | |
| EVA003 | ESCA | |
| (SEQ ID | | |
| NO. 3) | | |
| EVA004, | ESCA | oesophageal, head and |
| (SEQ ID | | neck cancer, melanoma, |
| NO. 4) | | pancreatic, lung, |
| | | colorectal |
| EVA005 | H&N, ESCA, LALM, STAD | Oesophageal, head and |
| (SEQ ID | | neck, melanoma, lung, |
| NO. 5) | | colorectal, pancreatic, |
| | | breast, bladder, renal, |
| | | ovarian |
| EVA006 | BRCA, CHOL, ESCA, LUAD, | Oesophageal, breast, |
| (SEQ ID | LUSC, OV, PAAD, STAD | bladder, colorectal, |
| NO. 6) | | lung, pancreatic, |
| | | head and neck, ovarian |
| EVA007 | ESCA, HNSC, LUSC | Oesophageal, head and |
| (SEQ ID | | neck, melanoma, |
| NO. 7) and | | pancreatic, lung, |
| EVA008 | | colorectal, breast, |
| (SEQ ID | | bladder |
| NO. 8) | | |
| EVA009, | LUAD, KIRC, ESCA | |
| (SEQ ID | | |
| NO. 9) | | |
| EVA010, | HNSC, LUAD, LUSC | |
| (SEQ ID | | |
| NO. 10) | | |
| EVA011 | STAD, MESO, LUSC | Oesophageal, pancreatic, |
| (SEQ ID | | head and neck, lung, |
| NO. 11) | | colorectal, bladder |
| EVA012, | COAD, LUAD, STAD | |
| (SEQ ID | | |
| NO. 12) | | |
| EVA013 | H&N, LUSC, ESCA | Oesophageal, head and |
| (SEQ ID | | neck, melanoma, lung, |
| NO. 13) | | breast, bladder. |
| EVA014 | H&N, LUSC, ESCA | |
| (SEQ ID | | |
| NO. 14) | | |
| EVA015 | LCA, CESC, COAD, ESCA, | oesophageal, head and |
| (SEQ ID | HNSC, LUSC, OV, READ, | neck cancer, melanoma, |
| NO. 15) | SARC, STAD, TGCT | ovarian, lung, |
| | | colorectal |
| EVA016, | ESCA | |
| (SEQ ID | | |
| NO. 16) | | |
| EVA017, | HNSC, CESC, LUSC | |
| (SEQ ID | | |
| NO. 17) | | |
| EVA018 | BRCA, ESCA, LUAD, PAAD, | |
| (SEQ ID | STAD | |
| NO. 26) | | |

In one embodiment, the invention provides a polypeptide, nucleic acid, vector or composition of the invention for use in medicine.

Further aspects of the invention relate to a method of raising an immune response in a human which comprises administering to said human the polypeptide, nucleic acid, vector or composition of the invention.

The present invention also provides a polypeptide, nucleic acid, vector or composition of the invention for use in raising an immune response in a human.

There is also provided the use of a polypeptide, nucleic acid, vector or composition of the invention for the manufacture of a medicament for use in raising an immune response in a human.

Suitably the immune response is raised against a cancerous tumor expressing a corresponding sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof. By “corresponding” in this context is meant that if the tumor expresses, say, any of SEQ ID NOs. 1 to 17 and 57 or a variant, or fragment, such as an immunogenic fragment, thereof as disclosed herein (SEQ ID NOs. 18 to 56 and 58 or variant thereof) then the polypeptide, nucleic acid, vector or composition of the invention and medicaments involving these will be based on any of SEQ ID NOs. 1 to 17 and 57 or a variant or fragment, such as an immunogenic fragment, thereof, for example a sequence comprising or consisting of the sequence selected from any one of SEQ ID NOs. 18 to 56 and 58 and a variant sequence thereof.

Suitably the immune response comprises an immune response in a immunoresponsive cell, for example a CD8+ T-cell, or a CD4+ T-cell and/or an antibody response, particularly CD8+ cytolytic T-cell response and/or a CD4+ helper T-cell response. As disclosed the immune response may comprise any of cell proliferation, cytokine or granzyme production and induction of tumour cell killing or apoptosis.

Suitably the immune response is raised against a tumor or tumour cell, particularly one expressing a sequence selected from any of SEQ ID NOs. 1 to 17 and 57 variants thereof and fragments, such as immunogenic fragments, thereof as disclosed herein (SEQ ID NOs. 18 to 56 and 58 or variant thereof).

In a preferred embodiment, the tumor may be any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC, for example wherein:
The ovarian cancer can comprise any of ovarian carcinoma, serous types such as ovarian serous cystadenocarcinoma;
The colon cancer can comprise any of adenocarcinoma, which may be mesenchymal or mesenchymal chondrosarcomas, gastrointestinal stromal tumors (GIST), lymphoma, carcinoids turcot syndrome, Peutz-Jeghers syndrome (PJS), familial colorectal cancer (FCC), juvenile polyposis coli;
The esophageal cancer can comprise any of squamous cell carcinoma, which often starts in squamous cells that line the esophagus and usually develops in the upper and middle part of the esophagus, adenocarcinoma, which often starts in the glandular tissue in the lower part of the esophagus where the esophagus and the stomach come together, small cell carcinoma, and/or may comprise esophageal adenocarcinoma (EAC) or esophageal squamous cell carcinoma (ESSC);
The breast cancer can comprise any of carcinoma, lobular carcinoma in situ (LCIS), angiosarcomas of the breast, Paget's disease, inflammatory breast cancer, invasive breast cancer, triple negative breast cancer, invasive lobular breast cancer, metastatic breast cancer, medullary breast cancer, mucinous (mucoid or colloid) breast cancer, tubular breast cancer, adenoid cystic carcinoma of the breast, metaplastic breast cancer, lymphoma of the breast, basal type breast cancer, phyllodes or cystosarcoma phyllodes, papillary breast cancer;
The skin cancer may comprise any of basal cell carcinoma, squamous cell carcinoma and melanoma, for example where the melanoma is cutaneous melanoma or uveal melanoma, particularly cutaneous melanoma.

The gastrointestinal tract or GI tract includes any of the; mouth, pharynx, esophagus, stomach, small intestine, duodenum, jejunum, and ileum, the large intestine including the colon starting at the cecum and ending at the rectum and anal canal, additionally it includes the gastrointestinal tract accessory organs of digestion the tongue, salivary glands, pancreas, liver and gallbladder. Thus gastrointestinal (GI) cancer includes cancer of any of the foregoing structures, most particularly this includes either of colorectal cancer/tumour and esophageal cancer/tumour, for example either of colorectal adenocarcinoma and esophageal adenocarcinoma. Gastrointestinal cancer can also include gastrointestinal stromal tumor (GIST), often located in the small intestine or stomach which may comprise form cancer in the soft mesenchymal tissue of the gastrointestinal tract and of the interstitial cells of Cajal (ICCs).

Stromal cancer includes any of the stromal rich cancer types for example any of gastrointestinal stromal tumor (GIST), pancreatic cancer, prostate cancer, breast cancer.

Prostate cancer includes adenocarcinoma of the prostate, transitional cell carcinoma of the prostate, squamous cell carcinoma of the prostate and small cell prostate cancer.

Pancreatic cancer may be either exocrine, endocrine or neuroendocrine cancer or islet tumors, for example pancreatic cancer may be adenocarcinoma or exocrine adenocarcinoma, pancreatic cancer may further include any of; mucinous cystic neoplasm, intraductal papillary-mucinous neoplasm (IPMN), and acinar cell carcinoma. Pancreatic endocrine cancer types include any of: gastrinoma, glucaganoma, Insulinoma, somatostatinoma, VIPoma (VIP=vasoactive intestinal peptide), nonfunctional islet cell tumor or cancer.

Head and neck cancer can comprise any of cancer of the hypopharynx, nasopharynx, oropharynx, paranasal sinus and nasal cavity, salivary gland, oral cavity, larynx, melanoma of the head and neck, oral cavity and oropharyngeal cancer, basal cell cancer of the head and neck, squamous cell cancer of the head and neck, the cancer may be squamous cell carcinoma (SCC) or sarcoma. Sarcoma can comprise any of, Kaposi sarcoma, liposarcoma, osteosarcoma, Ewing sarcoma, soft tissue sarcoma.

Rectal cancer can comprise any of rectal: adenocarcinoma, signet ring cell adenocarcinoma, mucinous adenocarcinoma, adenosquamous carcinoma (ASC), neuroendocrine cancer, small cell carcinoma, melanoma, squamous cell carcinoma, carcinoid tumors, gastrointestinal stromal tumor, lymphoma and hereditary rectal cancer.

Lung cancer can comprise any of lung squamous cell carcinoma (LUSC), lung adenocarcinoma (LUAD), large-cell carcinoma. small-cell lung cancer, small cell lung cancer (SCLC), non small cell lung cancer (NSCLC), adenosquamous carcinoma, sarcomatoid carcinoma, lung sarcoma, lung lymphoma

Stomach cancer, can comprise any of stomach adenocarcinoma, gastrointestinal stromal tumor (GIST), carcinoid tumor, lymphoma, neuroendocrine tumor, carcinoid tumor, squamous cell carcinoma, gastric lymphoma, leiomyosarcoma.

Cervical cancer, can comprise any of cervical squamous cell carcinoma or adenocarcinoma.

Uterine cancer can comprise any of endometrial cancer, and uterine sarcoma, clear-cell adenocarcinoma, serous adenocarcinoma, [symbol] mucinous neoplasm, small-cell carcinoma, [symbol] endometrial stromal sarcoma, carcinosarcoma, endometrioid tumor, uterine carcinosarcoma, squamous cell carcinoma.

The tumor or cancer may be primary or metastatic.

Further aspects of the invention relate to a method of treating a human patient suffering from cancer wherein the cells of the cancer express a sequence selected from any of SEQ ID NOs. 1 to 17 and 57 and variants and fragments, such as immunogenic fragments, thereof, or of preventing a human from suffering from cancer which cancer would express a sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof, which method comprises administering to said human a corresponding polypeptide, nucleic acid, vector or composition of the invention.

The present invention also provides a polypeptide, nucleic acid, vector or composition of the invention for use in treating or preventing cancer in a human, wherein the cells of the cancer express a corresponding sequence selected from any of SEQ ID NOs. 1 to 17 and 57 and variants and fragments, such as immunogenic fragments, thereof as described herein. The cancer or tumor may be any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

In this and the following description of the various aspects and embodiments of the present invention, particularly those therapeutic embodiments and aspects, fragments of polypeptides of any of SEQ ID NOs. 1 to 17 and 57, such as immunogenic fragments thereof, and thus example peptides of the invention, include polypeptides which comprise or consist of a sequence selected from any one of SEQ ID NOs. 18 to 56 and 58 and a variant sequence thereof, for example, any one of SEQ ID NOs. 18 to 56 and 58 or variants or immunogenic variants of any one of SEQ ID NOs. 18 to 56 and 58, for example that have 1, 2, or 3, amino acid variations selected from additions, substitutions and deletions with respect thereto.

The words “prevention” and “prophylaxis” are used interchangeably herein.

### Treatment and Vaccination Regimes

A therapeutic regimen may involve either separate, simultaneous (such as co-administration) or sequential (such as a prime-boost) delivery of (i) a polypeptide, nucleic acid or vector of the invention with (ii) one or more further polypeptides, nucleic acids or vectors of the invention and/or (iii) a further component such as a variety of other therapeutically useful compounds or molecules such as antigenic proteins optionally simultaneously administered with adjuvant. Examples of co-administration include homo-lateral co-administration and contra-lateral co-administration. “Simultaneous” administration suitably refers to all components being delivered during the same round of treatment. Suitably all components are administered at the same time (such as simultaneous administration of both DNA and protein), however, one component could be administered within a few minutes (for example, at the same medical appointment or doctor's visit) or within a few hours.

In some embodiments, a “priming” or first administration of a polypeptide, nucleic acid or vector of the invention, is followed by one or more “boosting” or subsequent administrations of a polypeptide, nucleic acid or vector of the invention (“prime and boost” method). In one embodiment the polypeptide, nucleic acid or vector of the invention is used in a prime-boost vaccination regimen. In an embodiment both the prime and boost are of a polypeptide of the invention, the same polypeptide of the invention in each case. In an embodiment both the prime and boost are of a nucleic acid or vector of the invention, the same nucleic acid or vector of the invention in each case. Alternatively, the prime may be performed using a nucleic acid or vector of the invention and the boost performed using a polypeptide of the invention or the prime may be performed using a polypeptide of the invention and the boost performed using a nucleic acid or vector of the invention. Usually the first or “priming” administration and the second or “boosting” administration are given about 1-12 weeks later, or up to 4-6 months later. Subsequent “booster” administrations may be given as frequently as every 1-6 weeks or may be given much later (up to years later).

### Antigen Combinations

According to the present invention the polypeptides, nucleic acids or vectors of the invention can be used in combination with one or more other polypeptides or nucleic acids or vectors of the invention (for example any of 1, 2, 3, 4, 5, 6, 7, 8, 9, 10, 11 or 12) and/or with one or more other antigenic polypeptides or polynucleotides or vectors encoding them, (for example any of 1, 2, 3, 4, 5, 6, 7, 8, 9, 10, 11 or 12) which cause an immune response to be raised against any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC. These other antigenic polypeptides could be derived from diverse sources, they could include well-described cancer associated antigens or cancer tumor-associated antigens (TAAs) for the aforementioned cancer or tumour types, such as MUC16, CRABP1/2, FOLR1 and KLK10, CEA, MAGE-A1, MAGE-A3, MAGE-A4, PRAME, hTERT, HER2, MUC1, Survivin, STEAP1, SOX2, NY-ESO-1, LAGE-1, OIP5, TTK, PLU1, DKKL1, FBXO39, AFP, SSX, EGFR, hTERT, ALDH, IDO, PSMA, VEGF, TEM-1 or antigenic peptide dervived therefrom.

Certain cancer associated antigens or cancer tumour associated antigens (TAAs) are known to be associated with particular cancer types, for example: Oesophageal cancer TAAS include: TP53, CDKN2A, and PIK3CA, NOTCH1, MUC16, EPHA2, and CTNNB1 and peptides derived therefrom. Head and neck cancer TAAS include: TP53, NOTCH1, HRAS, CASP8, FAT1, HPV E6 and E7 polypeptides and peptides derived therefrom. Lung cancer TAAS include: ALK fusion-derived peptides, EGFR, KRAS, ATM, BRCA1/2, RB1, TP53, NOTCH1, PIK3CA, BRAF, ALK, EML4-ALK fusion, MET, STK11, KEAP1 and peptides derived therefrom. Colorectal cancer TAAS include: APC, KRAS, TP53, BRAF, PIK3CA, TGFBR2, SMAD4, MLH1, MSH2, MSH6, PMS2 and peptides derived therefrom. Pancreatic cancer TAAS include: TMOD3, TPX2, WNT7A, CA19-9, MUC16CD, ADAM9, EFNB2 and peptides derived therefrom. Breast cancer TAAS include: TP53, RB1, and PTEN, TP53, PIK3CA, GATA3, BRCA1/BRCA2, ERBB2 (HER2), AKT1, ESR1 and peptides derived therefrom. Bladder cancer TAAS include: TP53, FGFR3, PIK3CA, HRAS, KRAS, RB1, ERBB2 (HER2), TSC1, ARID1A, KDM6A, STAG2 and peptides derived therefrom. Renal cancer TAAS include: VHL, PBRM1, SETD2, BAP1, MTOR, PIK3CA, PTEN, TP53, KDM5C and peptides derived therefrom. Ovarian cancer TAAS include: TP53, BRCA1/BRCA2, PIK3CA, KRAS, CTNNB1, ARID1A, NF1, PTEN, RB1, NOTCH3 and peptides derived therefrom. Melanoma cancer TAAS include: BRAF, NRAS, KIT, TP53, CDKN2A, PTEN, NF1, RAC1, TERT and peptides, e.g. antigenic peptides, derived therefrom. Any one or more of these foregoing TAAs and/or TAAs known to be associated with particular cancer types may be combined with a polypeptide of the invention in an embodiment of a fusion protein or in an embodiment of a combination (e.g. for co-administration) of the present invention, for example particularly where said TAA and said polypeptide of the invention are disclosed herein as associated with the same cancer type (see Table 1, herein), e.g. the combination or fusion protein thereby being suitable for the treatment or prevention of said cancer type or the raising of an immune response against a cancerous tumour of said cancer type for example expressing a sequence of the polypeptide of the invention.

In addition, combination or fusion protein of the invention and/or the antigenic peptides from these sources (cancer associated antigens) could also be combined with (i) non-specific immunostimulant/adjuvant species and/or (ii) an antigen, e.g. comprising universal CD4 helper epitopes, known to elicit strong CD4 helper T cells (delivered as a polypeptides, or as polynucleotides or vectors encoding these CD4 antigens), to amplify the anti-cancer-specific responses elicited by co-administered antigens. Examples of universal CD4+ helper epitopes include: PADRE (Pan-DR Epitope, sequence AKFVAAWTLKAAA, SEQ ID NO:59), HA307-319 (hemagglutinin epitope from Influenza A, sequence: PKYVKQNTLKLAT, SEQ ID NO:60), TT830-843 (tetanus toxin epitope, sequence: QYIKANSKFIGITE, SEQ ID NO:61), VP2 (epitope from VP2 capsid protein, sequence: FNNFTVSFWLRVPKVSASHLE, SEQ ID NO: 62), and ovalbumin-derived (OVA323-339, sequence: ISQAVHAAHAEINEAGR, SEQ ID NO:63).

Different polypeptides, nucleic acids or vectors may be formulated in the same formulation or in separate formulations, for example for use as a combination. Alternatively, polypeptides may be provided as fusion proteins in which a polypeptide of the invention is fused to a second or further polypeptide (such as any of the foregoing TAAs and/or any one or more further different polypeptides of the invention). For example where the further polypeptide is a cancer associated antigen, for example a protein or polypeptide or antigen associated with for example any one or more of an ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC associated protein or polypeptide or antigen, for example any one or more of MUC16, CRABP1/2, FOLR1 and KLK10, CEA, MAGE-A1, MAGE-A3, MAGE-A4, PRAME, hTERT, HER2, MUC1, Survivin, STEAP1, SOX2, NY-ESO-1, LAGE-1, OIP5, TTK, PLU1, DKKL1, FBXO39, AFP. SSX, EGFR, hTERT, ALDH, IDO, PSMA, VEGF, TEM-1 or antigenic peptide derived therefrom and/or any one or more foregoing disclosed TAA or TAA known to be associated with particular cancer types or antigenic peptide derived therefrom.

Nucleic acids may be provided which encode the aforementioned fusion proteins.

More generally, when two or more components are utilised in combination, the components could be presented, for example:
(1) as two or more individual antigenic polypeptide components;
(2) as a fusion protein comprising both (or further, i.e. two or more) polypeptide components;
(3) as one or more polypeptide and one or more polynucleotide component;
(4) as two or more individual polynucleotide components;
(5) as a single polynucleotide encoding two or more individual polypeptide components; or
(6) as a single polynucleotide encoding a fusion protein comprising both (or further, i.e. two or more) polypeptide components.

For convenience, it is often desirable that when a number of components are present they are contained within a single fusion protein or a polynucleotide encoding a single fusion protein (see below). In one embodiment of the invention all components are provided as polypeptides (e.g., within a single fusion protein). In an alternative embodiment of the invention all components are provided as polynucleotides (e.g., a single polynucleotide, such as one encoding a single fusion protein).

### Fusion Proteins

As an embodiment of the above discussion of antigen combinations, the invention also provides an isolated polypeptide according to the invention fused to a second or further polypeptide of the invention (for example any of 1, 2, 3, 4, 5, 6, 7, 8, 9, 10, 11 or 12), (herein after “fusion protein” or a “combination polypeptide of the invention”), optionally wherein each polypeptide is different. Such combination polypeptides of the invention can be produced by creating nucleic acid constructs that fuse together the sequences encoding the individual antigens. Combination polypeptides of the invention are expected to have the utilities described herein for polypeptides of the invention, and may have the advantage of superior immunogenic or vaccine activity or prophylactic or therapeutic effect (including increasing the breadth and depth of responses in-vitro, for example in a cancer cell, or in-vivo, for example in a subject suffering from cancer), and may be especially valuable in an outbred population. Fusions of polypeptides of the invention may also provide the benefit of increasing the efficiency of construction and manufacture of vaccine antigens and/or vectored vaccines (including nucleic acid vaccines).

As described above in the Antigen Combinations section, polypeptides of the invention and combination polypeptides of the invention may also be fused to polypeptide sequences which are not polypeptides of the invention, including one or more of (for example any of 1, 2, 3, 4, 5, 6, 7, 8, 9, 10, 11 or 12 of) the polypeptides selected from:
(a) other polypeptides which are cancer associated antigens (as herein defined) and thus potentially useful as immunogenic sequences in a vaccine (e.g., selected from any one or more of MUC16, CRABP1/2, FOLR1, KLK10, CEA, MAGE-A1, MAGE-A3, MAGE-A4, PRAME, hTERT, HER2, MUC1, Survivin, STEAP1, SOX2, NY-ESO-1, LAGE-1, OIP5, TTK, PLU1, DKKL1, FBXO39, AFP, SSX, EGFR, hTERT, ALDH, IDO, PSMA, VEGF, TEM-1 or antigenic peptide dervived therefrom referred to supra) and/or any one or more cancer associated antigens (TAA) or cancer associated antigens known to be associated with particular cancer types or antigenic peptide derived therefrom as disclosed herein; and
(b) polypeptide sequences which are capable of enhancing an immune response (i.e. immunostimulant sequences), and
(c) polypeptide sequences, e.g. comprising universal CD4 helper epitopes, which are capable of providing strong CD4+ help to increase CD8+ T cell responses to CST antigen epitopes.

Examples of immunostimulant sequences include: thymosin Alpha-1 (Tα1), muramyl dipeptide (MDP) and muramyl dipeptide derivative FK-565, poly-L-arginine, beta-defensin, BAMBI-derived peptides, P40 peptide, epitalon. The invention also provides nucleic acids encoding the aforementioned fusion proteins and other aspects of the invention (vectors, compositions, cells etc) mutatis mutandis as for the polypeptides of the invention.

### CST Antigen-Binding Polypeptides

Antigen-binding polypeptides which are immunospecific for tumor-expressed antigens (polypeptides of the invention) may be designed to recruit cytolytic cells to antigen-decorated tumor cells, mediating their destruction. One such mechanism of recruitment of cytolytic cells by antigen-binding polypeptides is known as antibody-dependent cell-mediated cytotoxicity (ADCC). Thus the invention provides an antigen-binding polypeptide which is immunospecific for a polypeptide of the invention, optionally in complex with (i.e. bound to) HLA as described herein. Antigen-binding polypeptides include antibody derived molecules for example antibodies such as monoclonal antibodies and fragments, particularly antigen binding fragments, thereof e.g., domain antibodies, Fab fragments, Fv fragments, for example scFv, and VHH fragments which may be produced in a non-human animal species (e.g., rodent or camelid) and humanised or may be produced in a non-human species (e.g., rodent genetically modified to have a human immune system).

Antigen-binding polypeptides, which may be antibody derived molecules, may be produced by methods well known to a skilled person. For example, monoclonal antibodies can be produced using hybridoma technology, by fusing a specific antibody-producing B cell with a myeloma (B cell cancer) cell that is selected for its ability to grow in tissue culture and for an absence of antibody chain synthesis (Köhler and Milstein, 1975, Nature 256 (5517): 495-497 and Nelson et al., 2000 (Jun), Mol Pathol. 53 (3): 111-7 herein incorporated by reference in their entirety).

A monoclonal antibody directed against an antigen (for example a CST antigen of the invention) can, for example, be obtained by:
a) immortalizing lymphocytes obtained from the peripheral blood of an animal (including a human) previously immunized/exposed with a determined antigen, with an immortal cell and preferably with myeloma cells, in order to form a hybridoma,
b) culturing the immortalized cells (hybridoma) formed and recovering the cells producing the antibodies having the desired specificity.

Monoclonal antibodies can be obtained by a process comprising the steps of:
a) cloning into vectors, especially into phages and more particularly filamentous bacteriophages, DNA or cDNA sequences obtained from lymphocytes especially peripheral blood lymphocytes of an animal (suitably previously immunized with determined antigens),
b) transforming prokaryotic cells with the above vectors in conditions allowing the production of the antibodies,
c) selecting the antibodies by subjecting them to antigen-affinity selection,
d) recovering the antibodies having the desired specificity
e) expressing antibody-encoding nucleic acid molecules, or nucleic acids encoding antigen binding fragments thereof, obtained from B cells of patients exposed to antigens, or animals experimentally immunized with antigens.

The selected antibodies, or antigen binding fragments thereof, may then be produced using conventional recombinant protein production technology (e.g., from genetically engineered cells, e.g. HEK 298 cells or CHO cells).

The invention provides an isolated antigen-binding polypeptide which is immunospecific for a polypeptide of the invention. Suitably, the antigen-binding polypeptide is a monoclonal antibody or a fragment thereof, for example an antigen binding fragment thereof, for example any of Fab, Fab′, diabody, tribody, minibody, scFv, scFv-Fc, F (ab′) 2, VhH, V-NAR, dab, dab-Fc, free-LC, half antibody, preferably for example an scFv. The term immunospecific refers to the ability of an antigen binding polypeptide such as an antibody derived molecule, e.g. antibody or antigen binding fragment thereof or a T cell receptor derived molecule, e.g. a T cell receptor, or antigen binding fragment thereof, to bind or specifically bind to a specific antigen or epitope, e.g. a MHC presented peptide antigen or polypeptide according to the invention, and not with unrelated antigen or peptide molecules and optionally induce an immune response in the respective B-cell or T-cell/NK-cell producing/presenting said antigen binding polypeptide. The same definition would apply to the respective B-cell or T-cell producing said antigen binding polypeptide.

In certain embodiments, the antigen-binding polypeptide is coupled to a cytotoxic moiety. Example cytotoxic moieties include the Fc domain of an antibody, which will recruit Fc receptor-bearing cells facilitating ADCC. Alternatively, the antigen-binding polypeptide may be linked to a biological toxin, or a cytotoxic chemical, for example calicheamicin, doxorubicin or monomethyl auristatin E.

Another important class of antigen-binding polypeptide includes T-cell receptor (TCR)-derived molecules that bind to HLA-displayed polypeptides, fragments or variants of the present invention for example HLA-displayed fragments of the antigens of this invention. In this embodiment, TCR-based biologicals or T-cell receptor (TCR)-derived molecules (including TCRs such as TCRs derived directly from subjects or patients, or specifically manipulated, high-affinity TCRs for example recombinant TCRs or T-cell receptor (TCR)-derived molecules) that recognize CST antigens (or derivatives or variants thereof) on the surface of tumor cells, e.g. HLA-displayed polypeptides, fragments or variants of the present invention.

Accordingly, an antigen-binding polypeptide according to the invention may be a T cell receptor or TCR for example that comprises an α chain and a β chain for example where the extracellular region of each chain comprises three CDRs (CDR1, CDR2, CDR3) and four framework regions which are either side of the CDRs, and a constant region. Each chain has a connecting peptide region which links the transmembrane and intracellular regions to the extracellular domain at its C terminus. The TCR may also be a soluble form of the TCR for example which consists of the extracellular domain (also called the antigen-binding domain). The soluble TCR may lack the transmembrane and, ideally also, intracellular domain, for example it may consist of the a chain and a β chain variable domains optionally comprising respective constant domains or parts thereof, alternatively lacking constant domains, for example where the a chain and β chain variable domains are linked to form a single chain variable domain, scTv.

An antigen binding polypeptide of the invention which is a TCR derived molecule may be produced by methods well known in the art. For example, (a) isolating T-cells from a sample of peripheral blood, (ii) contacting the T-cells with a polypeptide of the invention, for example as presented on an antigen presenting cell, e.g. dendritic cell or on a multimeric MHC reagent, e.g. monomer, dextramer, pentamer reagent, (iii) identifying and isolating a T-cell which expresses a TCR which specifically binds to and/or the binding of which elicits an immune response in said T-cell (e.g. induction of T-cell proliferation and/or production of cytokines, e.g. IFN-gamma, IL-2, TNF-alpha or for example granzyme and/or immunoresponsive cell induction of tumour cell killing or apoptosis), (iv) isolating the nucleic acid encoding said TCR and/or antigen binding fragment thereof and optionally (v) transfecting said nucleic acid into a cell, e.g. CHO cell, KED 293 cell, T-cell or Jurkat cell, such that the TCR or antigen binding fragment thereof is expressed, optionally expressed at the cell surface or secreted as a soluble TCR derived molecule, further optionally purifying said soluble molecule.

Thus, in an embodiment, the antigen-binding polypeptide is immunospecific for and/or binds to an HLA-bound polypeptide that is or is part of a polypeptide of the invention, i.e. a polypeptide of the invention, variant, immunogenic fragment thereof. For example, the antigen-binding polypeptide is a recombinant T-cell receptor or a chimeric antigen receptor (CAR) or an antigen-binding fragment thereof.

In an embodiment, an antigen-binding polypeptide of the invention (which may comprise an antibody derived molecule or a TCR derived molecule) may be coupled to an immune cell activating component or ligand, for example another polypeptide (for example an antibody or binding domain of an antibody, for example a Fab or Fab fragment, Fv or Fv fragment, for example scFv) that is capable of binding to immunoresponsive cells, e.g. T-cells or cytotoxic cells or cytotoxic T-cells or other immune components such as cell surface receptors of said cells such as CD3, CD4, CD8, CD70, CD137, CD28, preferably CD28 or CD3, more preferably CD3 in a subject, preferably the immune cell activating component or ligand is an antibody derived polypeptide which binds to an immune component, preferably, CD3.

Accordingly, the antigen-binding polypeptide may be a TCR-based biological comprising a T-cell receptor (TCR)-derived molecule; for example a TCR or antigen binding fragment thereof including TCRs such as TCRs derived directly from subjects or patients, or specifically manipulated, high-affinity TCRs for example recombinant TCRs or T-cell receptor (TCR)-derived molecules or an antibody mimic thereof (i.e. antibody derived molecule, antibody or antibody fragment or binding domain as described herein that is immunospecific for and/or binds an HLA-bound polypeptide that is or is part of a polypeptide of the invention), that recognises CST antigens (or derivatives thereof) on the surface of tumor cells and may also include or be coupled to a targeting moiety or immune cell activating component or ligand which recognizes a T-cell or a component on a T cell (or another class of immune cell) that attract these immune cells to tumors, providing therapeutic benefit, for example the targeting moiety may be provided as antibody or antibody fragment or binding domain, particularly an Fv or scFv, e.g. that bind CD3. In some embodiments, the targeting moiety may also stimulate beneficial activities (including cytolytic activities) of the redirected immune cells.

For example according to this embodiment, there is provided a bispecific molecule or bispecific construct comprising the antigen-binding polypeptide which may for example be a TCR derived molecule, TCR or cancer-specific binding fragment (e.g. antigen binding fragment) of the TCR such as a soluble TCR, e.g. scTv, for example comprising TCR variable alpha and beta domains optionally with a constant alpha and/or beta domain or truncation thereof, or for example TCR Valpha-Vbeta domain or an scTv or antibody mimic of any thereof (i.e. antibody derived molecule, antibody or antibody fragment or binding domain that binds to the HLA presented antigen or peptide of the invention), which is coupled to an immune cell activating component or ligand (such as an antibody protein or binding domain of an antibody, e.g. scFv, Fv domain, or Fab) that binds to and activates an immune cell, for example such as T-cell e.g. a CD4+ or CD8+ T-cell. For example, the immune cell activating component or ligand activates an immune cell via binding to CD3. For example, one particular bispecific molecule or construct may comprise the polypeptide sequences of the TCR or cancer-specific binding fragment/antigen binding fragment of the invention, for example a soluble TCR or scTv (or antibody mimic thereof), and an antibody protein (particularly an agonist antibody protein) that binds to CD3, for example an scFv, Fv domain, or Fab. In an embodiment, there is provided a bispecific construct/molecule that may comprise the antigen-binding polypeptide which may for example be a TCR or cancer-specific binding fragment/antigen binding fragment of the TCR such as a soluble TCR, for example a TCR Valpha-Vbeta domain or scTv or antibody mimic thereof (i.e. antibody derived molecule, antibody or antibody fragment or binding domain that binds to the HLA presented antigen or polypeptide/peptide of the invention), and an immune cell activating component or ligand (such as an antibody derived molecule, antibody protein, particularly an Fv domain, Fab or scFv domain) that binds to and activates an immune cell, for example such as a T-cell e.g. a CD8+ T-cell. For example, the immune cell activating component or ligand activates an immune cell via binding to CD3. For example, one particular bispecific construct/molecule may comprise the polypeptide sequences of the TCR or cancer-specific binding fragment/antigen binding fragment of the invention, for example a soluble TCR of the invention, for example a TCR Valpha-Vbeta domain or scTv, and an immune cell activating component or ligand, e.g. an antibody protein, particularly an Fv domain or scFv domain or Fab (particularly an agonist antibody protein) that binds to CD3. The antibody protein comprising the immune cell activating component is suitably an antigen-binding domain of an antibody such as a VH (variable heavy domain from a 4 chain antibody) or a VHH (variable heavy domain from a 2 chain (heavy chain only) antibody such as from Camelid, or is a scFv (i.e. a fusion protein comprising the variable regions of the light and heavy chains of an antibody optionally connected by a short (e.g. 10-25 amino acids) linker. A number of anti-CD3 agonist antibodies are available in the prior art. For example, blinatumomab which is an approved bispecific T-cell engager (BiTE) product consists of a CD19-targeting antibody (heavy chain scFv) connected to a CD3-targeting agonist antibody (light chain scFv). The bispecific molecule or construct according to the invention may further comprise an antibody Fc domain or part thereof.

A linker may be provided to link a chain of the TCR or cancer-specific binding fragment/antigen binding fragment thereof to the other part of the bispecific construct/molecule, e.g. the immune cell activating component. More generally, there is provided a fusion protein comprising the TCR derived molecule or TCR or cancer-specific/antigen binding fragment thereof (or an antibody mimic thereof, i.e. antibody derived molecule as described herein) of the invention and a heterologous protein providing the immune cell stimulating and/or activating activity, for example an antibody, which may be a monoclonal antibody, or antibody fragment or binding domain, for example any of Fab, Fab′, diabody, tribody, minibody, scFv, scFv-Fc, F (ab′) 2, VhH, V-NAR, dab, dab-Fc, free-LC, half antibody, optionally coupled by a linker. There is also provided a polynucleotide encoding said bispecific, bispecific construct/molecule or fusion protein. The linker may comprise a short polypeptide of between 5 and 20 amino acids comprising glycine and/or serine

In an embodiment, the antigen-binding polypeptide, which may be the bispecific construct or molecule of the invention, is for use in medicine.

In an embodiment, there is provided a pharmaceutical composition comprising an antigen-binding polypeptide of the invention, which may be the bispecific construct or molecule of the invention, together with a pharmaceutically acceptable carrier. Such a composition may be a sterile composition suitable for parenteral administration. See e.g., disclosure of pharmaceutical compositions supra.

There is provided by the invention a method of treating a human suffering from cancer wherein the cells of the cancer express a sequence selected from SEQ ID NO. 1 to 17 and 57 and variants and fragments, such as immunogenic fragments, thereof (for example a sequence comprising or consisting of the sequence selected from any one of SEQ ID NOs. 18 to 56 and 58 and a variant sequence thereof), or of preventing a human from suffering from cancer wherein the cells of the cancer would express a sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof which comprises administering to said human an antigen-binding polypeptide or composition comprising said antigen-binding polypeptide of the invention.

In an embodiment, there is provided an antigen-binding polypeptide of the invention, which may be coupled to a cytotoxic moiety, or composition comprising said antigen-binding polypeptide of the invention for use in treating or preventing cancer in a human, wherein the cells of the cancer express a corresponding sequence selected from any of SEQ ID NOs. 1 to 17 and 57 and fragments of any one thereof as herein described (for example a sequence comprising or consisting of the sequence selected from any one of SEQ ID NOs. 18 to 56 and 58 and a variant sequence thereof).

Suitably in any of the above embodiments, the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

Antigen-binding polypeptides (such as antibodies or fragments thereof may be administered at a dose of e.g. 5-1000 mg e.g. 25-500 mg e.g. 100-300 mg e.g. ca. 200 mg.

### Cell Therapies to Facilitate Antigen Presentation In Vivo

Any of a variety of cellular delivery vehicles may be employed within pharmaceutical compositions to facilitate production of an antigen-specific immune response. Thus the invention provides a cell which is an isolated antigen presenting cell modified by ex vivo loading with a polypeptide of the invention (e.g. by pulse said polypeptide of the invention onto the cells) or genetically engineered to express the polypeptide of the invention, for example by transduction or transfection with a nucleic acid or vector of the invention such that the encoded polypeptide is expressed and presented by the cell for example in complex with MHC, (herein after referred to as a “APC of the invention”). Antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response and/or to be immunologically compatible with the receiver (i.e., matched HLA haplotype). APCs may generally be isolated from any of a variety of biological fluids and organs, and may be autologous, allogeneic, syngeneic or xenogeneic cells.

Certain preferred embodiments of the present invention use dendritic cells or progenitors thereof as APCs. Thus, in an embodiment, the APC of the invention is a dendritic cell. Dendritic cells are highly potent APCs (Banchereau & Steinman, 1998, Nature, 392:245-251) and have been shown to be effective as a physiological adjuvant for eliciting prophylactic or therapeutic immunity (see Timmerman & Levy, 1999, Ann. Rev. Med. 50:507-529). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course be engineered to express specific cell-surface receptors or ligands that are not commonly found on dendritic cells in vivo or ex vivo, and such modified dendritic cells are contemplated by the present invention. As an alternative to dendritic cells, antigen-loaded secreted vesicles (called exosomes) may be used within an immunogenic composition (see Zitvogel et al., 1998, Nature Med. 4:594-600). Thus, in an embodiment, there is provided an exosome loaded with a polypeptide of the invention. Antigen loaded secreted vesicles (exosome) may be produced by mixing isolated exosomes with the polypeptide or nucleic acid or vector of the invention, passing said mixture through a lipid extruder of 100-400 nm membrane or by means of vector transfection into said exosome.

Dendritic cells and progenitors may be obtained from peripheral blood, bone marrow, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNF to cultures of monocytes harvested from peripheral blood. Alternatively, CD34-positive cells harvested from peripheral blood, umbilical cord blood or bone marrow may be differentiated into dendritic cells by adding to the culture medium combinations of GM-CSF, IL-3, TNF□, CD40 ligand, LPS, flt3 ligand and/or other compound(s) that induce differentiation, maturation and proliferation of dendritic cells.

Dendritic cells are conveniently categorised as “immature” and “mature” cells, which allows a simple way to discriminate between two well-characterised phenotypes. However, this nomenclature should not be construed to exclude all possible intermediate stages of differentiation. Immature dendritic cells are characterised as APCs with a high capacity for antigen uptake and processing, which correlates with the high expression of Fc receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

According to the invention, APCs may also be genetically engineered e.g., transfected with a polynucleotide, for example a polynucleotide or vector according to the invention, encoding a protein (or portion or other variant thereof), for example a polypeptide according to the invention, such that the polypeptide is expressed on the cell surface for example in complex with MHC. Such transfection may take place ex vivo, and a pharmaceutical composition comprising such transfected cells may then be used, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., 1997, Immunology and Cell Biology 75:456-460. Antigen loading of dendritic cells may be achieved by incubating dendritic cells or progenitor cells with the polypeptide, DNA (e.g., a plasmid vector) or RNA; or with antigen-expressing recombinant bacteria or viruses (e.g., an adenovirus, adeno-associated virus (AAV) (e.g., AAV type 5 and type 2), alphavirus (e.g., Venezuelan equine encephalitis virus (VEEV), Sindbis virus (SIN), Semliki Forest virus (SFV)), herpes virus, arenavirus (e.g., lymphocytic choriomeningitis virus (LCMV)), measles virus, poxvirus (such as modified vaccinia Ankara (MVA) or fowlpox), paramyxovirus, lentivirus, or rhabdovirus (such as vesicular stomatitis virus (VSV)). Prior to polypeptide loading, the polypeptides may be covalently conjugated to an immunological partner that provides T cell help (e.g., a carrier molecule). Alternatively, a dendritic cell may be pulsed with a non-conjugated immunological partner, separately or in the presence of the polypeptide or vector.

The invention provides for delivery of specifically designed short, chemically synthesized epitope-encoded fragments of polypeptide antigens, for example polypeptides of the invention, to antigen presenting cells, for example a polypeptide according to the invention, for example a sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof. Those skilled in the art will realize that these types of molecules, also known as synthetic long peptides (SLPs) provide a therapeutic platform for using the antigenic polypeptides of this invention to stimulate (or load) cells in vitro (Gornati et al., 2018, Front. Imm, 9:1484), or as a method of introducing polypeptide antigen into antigen-presenting cells in vivo (Melief & van der Burg, 2008, Nat Rev Cancer, 8:351-60).

In an embodiment, there is provided a pharmaceutical composition comprising an antigen-presenting cell of the invention, which is suitably a dendritic cell, together with a pharmaceutically acceptable carrier. Such a composition may be a sterile composition suitable for parenteral administration. See e.g., disclosure of pharmaceutical compositions supra.

In an embodiment, there is provided an antigen-presenting cell of the invention, which is suitably a dendritic cell, for use in medicine.

There is also provided a method of treating a human suffering from cancer wherein the cells of the cancer express a sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof, or of preventing a human from suffering from cancer wherein the cells of the cancer would express a sequence selected from any of SEQ ID NOs. 1 to 17 and 57 and variants and fragments, such as immunogenic fragments, thereof, (for example a sequence comprising or consisting of the sequence selected from any one of SEQ ID NOs. 18 to 56 and 58 and a variant sequence thereof), which comprises administering to said human said antigen presenting cell of the invention, which is suitably a dendritic cell, or composition comprising said antigen presenting cell of the invention.

In an embodiment, there is provided an antigen presenting cell of the invention, which is suitably a dendritic cell, or composition comprising said antigen presenting cell of the invention for use in treating or preventing cancer in a human, wherein the cells of the cancer express a corresponding sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof.

In an embodiment, there is provided a pharmaceutical composition comprising an exosome of the invention together with a pharmaceutically acceptable carrier. Such a composition may be a sterile composition suitable for parenteral administration. See e.g., disclosure of pharmaceutical compositions supra. Compositions may optionally comprise immunostimulants-see disclosure of immunostimulants supra.

In an embodiment, there is provided an exosome comprising a polypeptide, nucleic acid, or vector according to the invention.

In an embodiment, there is provided an exosome of the invention for use in medicine.

There is also provided a method of treating a human suffering from cancer wherein the cells of the cancer express a sequence selected from any of SEQ ID NOs. 1 to 17 and 57 and variants and fragments, such as immunogenic fragments, thereof as described herein, (for example a sequence comprising or consisting of the sequence selected from any one of SEQ ID NOs. 18 to 56 and 58 and a variant sequence thereof), or of preventing a human from suffering from cancer wherein the cells of the cancer would express a sequence selected from any of SEQ ID NOs. 1 to 17 and 57 and variants and fragments, such as immunogenic fragments, thereof as herein described (for example a sequence comprising or consisting of the sequence selected from any one of SEQ ID NOs. 18 to 56 and 58 and a variant sequence thereof), which comprises administering to said human said exosome if the invention or composition comprising said exosome of the invention. In an embodiment, there is provided an exosome of the invention or composition comprising said exosome of the invention for use in treating or preventing cancer in a human, wherein the cells of the cancer express a corresponding sequence selected from any of SEQ ID NOs. 1 to 17 and 57 and fragments of any one thereof, (for example a sequence comprising or consisting of the sequence selected from any one of SEQ ID NOs. 18 to 56 and 58 and a variant sequence thereof).

In any one of the above embodiments, suitably the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

### Stimulated T-Cell Therapies

In addition to in vivo or ex vivo APC-mediated production of T-cells immunospecific for polypeptides of the invention, autologous or non-autologous T-cells may be isolated from a subject, e.g., from peripheral blood, umbilical cord blood and/or by apheresis, and stimulated in the presence of tumor-associated antigens which are loaded onto MHC molecules (signal 1) of APC cells, for example as herein described, to induce proliferation of T-cells with a TCR immunospecific for this antigen (optionally in the presence of anti-CD3 antibodies and anti-CD28 antibodies provide a co-stimulatory signal).

Successful T-cell activation requires the binding of the costimulatory surface molecules B7 and CD28 on antigen-presenting cells and T cells, respectively (signal 2). To achieve optimal T-cell activation, both signals 1 and 2 are required. Conversely, antigenic peptide stimulation (signal 1) in the absence of costimulation (signal 2) cannot induce full T-cell activation, and may result in T-cell tolerance. In addition to costimulatory molecules, there are also inhibitory molecules, such as CTLA-4 and PD-1, which induce signals to prevent T-cell activation.

Autologous or non-autologous T-cells may therefore be stimulated in the presence of a polypeptide or nucleic acid of the invention, and expanded and transferred back to the patient at risk of or suffering from cancer whose cancer cells express a corresponding polypeptide of the invention provided that the antigen-specific TCRs will recognize the antigen presented by the patient's MHC, where they will target and induce the killing of cells of said cancer which express said corresponding polypeptide.

In an embodiment, there is provided a polypeptide, nucleic acid, vector or composition of the invention for use in the ex vivo stimulation and/or amplification of T-cells derived from a human suffering from cancer, for subsequent reintroduction of said stimulated and/or amplified T cells into the said human for the treatment of the said cancer in the said human.

The invention provides a method of treatment of cancer in a human, wherein the cells of the cancer express a sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof, which comprises taking from said human a population of white blood cells comprising at least T-cells optionally with antigen-presenting cells, stimulating and/or amplifying said T-cells in the presence of a corresponding polypeptide, nucleic acid, vector or composition of the invention, and reintroducing some or all of said white blood cells comprising at least stimulated and/or amplified T-cells into the human.

In any one of the above embodiments, suitably the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

In an embodiment, there is provided a process for preparing a T-cell population which is cytotoxic for cancer cells which express a sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof which comprises (a) obtaining T-cells and antigen-presenting cells from a cancer patient and (ii) stimulating and amplifying the T-cell population ex vivo with a corresponding polypeptide, nucleic acid, vector or composition of the invention.

By “corresponding” in this context is meant that if the cancer cells express, for example, any of SEQ ID NOs. 1 to 17 and 57 or a variant or fragment, such as an immunogenic variant or fragment thereof as described herein, (for example a sequence comprising or consisting of the sequence selected from any one of SEQ ID NOs. 18 to 56 and 58 and a variant sequence thereof) then the T-cell population is stimulated and amplified ex vivo with any of (optionally the same) SEQ ID NOs. 1 to 17 and 57 or a variant or fragment, such as an immunogenic fragment as described herein, (for example a sequence comprising or consisting of the sequence selected from any one of SEQ ID NOs. 18 to 56 and 58 and a variant sequence thereof) thereof in the form of a polypeptide, nucleic acid or vector, or a composition containing one of the foregoing.

For example, in such processes, the culturing and expanding or stimulating and amplifying, may be performed in the presence of dendritic cells, for example an APC of the invention. The dendritic cells may be transfected with a nucleic acid molecule or with a vector of the invention and express a polypeptide of the invention or may be pulsed with said polypeptide as described herein.

The invention provides a T-cell population obtainable by any of the aforementioned processes (hereinafter a T-cell population of the invention).

In an embodiment, there is provided a cell which is a T-cell which has been stimulated with a polypeptide, nucleic acid, vector or composition of the invention (hereinafter a T-cell of the invention). Preferably said T-cell is immunoresponsive to the polypeptide, nucleic acid, vector or composition of the invention, preferably to the polypeptide of the invention, for example said T-cell may bind and display an “immune response”, for example the induction of T-cell proliferation and/or production of cytokines, e.g. IFN-gamma, IL-2, TNF-alpha and/or for example production of granzyme and/or induction of tumour cell killing or apoptosis.

In an embodiment, there is provided a pharmaceutical composition comprising a T-cell population or a T-cell of the invention together with a pharmaceutically acceptable carrier. Such a composition may, for example, be a sterile composition suitable for parenteral administration.

In an embodiment, there is provided a T-cell population or T-cell of the invention for use in medicine.

There is also provided a method of treating a human suffering from cancer wherein the cells of the cancer express a sequence selected from any of SEQ ID NOs. 1 to 17 and 57 and variants and fragments, such as an immunogenic fragments, thereof, for example a sequence comprising or consisting of the sequence selected from any one of SEQ ID NOs. 18 to 56 and 58 and a variant sequence thereof, or of preventing a human from suffering from cancer wherein the cells of the cancer would express a sequence selected from any of SEQ ID NOs. 1 to 17 and 57 and variants and fragments, such as immunogenic fragments, thereof, (for example a sequence comprising or consisting of the sequence selected from any one of SEQ ID NOs. 18 to 56 and 58 and a variant sequence thereof), as herein described which comprises administering to said human said T-cell population or T-cell of the invention or composition comprising said T-cell population or T-cell of the invention.

In an embodiment, there is provided a T-cell population of the invention, T-cell of the invention or composition comprising said T-cell population or T-cell of the invention for use in treating or preventing cancer in a human, wherein the cells of the cancer express a corresponding sequence selected from any of SEQ ID NOs. 1 to 17 and 57 and variants and fragments, such as immunogenic fragments, thereof as herein described, (for example a sequence comprising or consisting of the sequence selected from any one of SEQ ID NOs. 18 to 56 and 58 and a variant sequence thereof),

In any one of the above embodiments, suitably the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

The present invention further provides a process for preparing an immunoresponsive cell or population of immunoresponsive cells which binds to and/or elicits an immune response from, a polypeptide sequence according to the invention, for example a polypeptide sequence selected from any of SEQ ID NOs. 1 to 17 and 57 and variants and fragments thereof, such as immunogenic fragments thereof, for example a polypeptide sequence comprising or consisting of a sequence selected from any one of SEQ ID NOs. 18 to 56 and 58 or variants or immunogenic variants thereof, wherein the method comprises;
(a) obtaining an immunoresponsive cell or population of immunoresponsive cells,
(b) screening the immunoresponsive cell or population of immunoresponsive cells in a contacting step with the polypeptide sequence,
(c) identifying immunoresponsive cell or population of immunoresponsive cells which bind the polypeptide sequence, and optionally
(d) isolating the identified binding immunoresponsive cell or population of immunoresponsive cells.

According to one embodiment the variants or immunogenic variants of any of SEQ ID NOs. 1 to 17 and 57 or SEQ ID NOs: 18 to 56 and 58, have 1, 2, or 3, amino acid variations selected from additions, substitutions and deletions with respect thereto.

The present invention therefore provides an isolated immunoresponsive cell or population of immunoresponsive cells, which bind to the polypeptide sequence according to the invention and is/are produced according to the foregoing process of the present invention. Further optionally, the immunoresponsive cell or population of immunoresponsive cells which bind to the polypeptide sequence of the invention, may be T-cells or NKT cells, and/or the process may also comprise extracting or isolating nucleic acid encoding the T-cell receptor which binds to the polypeptide and further optionally involve transducing a further immunoresponsive cell or population of immunoresponsive cells to express the T-cell receptor. According to this embodiment the invention provides a modified immunoresponsive cell or population of immunoresponsive cells which express a T-cell receptor which binds to the polypeptide sequence according to the invention. Alternatively optionally, the immunoresponsive cell or population of immunoresponsive cells which bind to the polypeptide sequence of the invention, may be T-cells or NKT cells, and/or the process may also comprise the process may also comprise extracting or isolating nucleic acid encoding the T-cell receptor or antigen binding fragment thereof which binds to the polypeptide of the invention and further optionally involve transducing a host cell to express said TCR or fragment thereof preferably wherein said TCR or fragment is secreted as a soluble TCR as herein described.

The invention further provides the identified binding immunoresponsive cell or population of immunoresponsive cells or the modified immunoresponsive cell or population of immunoresponsive cells for use in treating cancer. In a preferred embodiment, the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC. The immunoresponsive cell or cells can be cells of the lymphoid lineage, for example peripheral blood mononuclear cells (PBMCs), white blood cells, or lymphocytes, comprising B, T or natural killer (NK) cells. The immunoresponsive cells may be cells of the lymphoid lineage including T cells, Natural Killer T (NKT) cells, and precursors thereof including embryonic stem cells, and pluripotent stem cells (e.g, those from which lymphoid cells may be differentiated). The T cells can include, but are not limited to, helper T cells, cytotoxic T cells, memory T cells (including central memory T cells, stem-cell-like memory T cells (or stem-like memory T cells), and two types of effector memory T cells: e.g. TEM cells and TEMRA cells, Regulatory T cells (also known as suppressor T cells), Natural Killer T cells, Mucosal associated invariant T cells, and gamma-delta T cells. Preferably, the immunoresponsive cell is a T cell optionally a CD4+ T cell or a CD8+ T cell. Accordingly, the immunoresponsive cells may be T-cells, optionally CD4+ T cells or CD8+ T cells, or the immunoresponsive cells may be a population of T-cells, optionally CD4+ T cells; or CD8+ T cells, or a mixed population of CD4+ T cells and CD8+ T cells.

Binding may be determined by known methods such as by equilibrium methods (e.g. enzyme-linked immunosorbent assay (ELISA) or radioimmunoassay (RIA)), or kinetics (e.g. BIACORE™ analysis). Binding to the polypeptide of the invention may be binding to a polypeptide presented by an antigen presenting cell, for example presented in complex with an antigen presenting molecule such as HLA, or binding to peptide in complex with an MHC tetramer or dextramer, for example chip or plate bound tetramer or dextramer, or binding to the free optionally labelled polypeptide.

### Engineered Immune Cell Therapies

Derivatives of all types of CST antigen-binding polypeptides described above, including antibody derived molecules, antibodies, TCR derived molecules, TCRs or TCR mimetics (see Dubrovsky et al., 2016, Oncoimmunology) that recognize CST antigen-derived peptides complexed to human HLA molecules, may be engineered to be expressed on the surface of immunoresponsive cells, e.g. T cells (autologous or non-autologous), which can then be administered as adoptive cell/T cell therapies to treat cancer.

These derivatives include for example “chimeric antigen receptors (CARs),” which, as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. CARs may direct specificity of the cell to a tumor associated antigen, a polypeptide of the invention, preferably wherein the polypeptide is HLA-bound.

Another approach to treating cancer in a patient is to genetically modify T-cells to target antigens expressed on tumor cells, via the expression of chimeric antigen receptors (CARs). This technology is reviewed in Wendell & June 2017, Cell, 168:724-740 (incorporated by reference in its entirety).

Yet another approach to treating cancer in a patient is to genetically modify T-cells to target antigens expressed on tumor cells, via the expression of recombinant T-cell receptors (TCRs) that target HLA-bound target antigens expressed on tumor cells, e.g. a polypeptide of the invention wherein the polypeptide is HLA-bound.

Such T-cells (including CAR T-cells) may be produced by the method of obtaining a sample of cells from the subject, e.g., from peripheral blood, umbilical cord blood and/or by apheresis, wherein said sample comprises T-cells or T-cell progenitors, and transfecting said cells with a nucleic acid encoding a T-cell receptor (for example, a recombinant TCR or a CAR) which is immunospecific for the polypeptide of the invention, wherein the polypeptide is HLA-bound. Such nucleic acid will be capable of integration into the genome of the cells, and the cells may be administered in an effective amount the subject to provide a T-cell response against cells expressing a polypeptide of the invention. For example, the sample of cells from the subject may be collected for example in a separate step.

It is understood that cells used to produce said recombinant TCR- or CAR-expressing T-cells may be autologous or non-autologous.

Transgenic CAR-expressing or TCR expressing T cells may have expression of an endogenous T-cell receptor and/or endogenous HLA inactivated. For example, the cells may be engineered to eliminate expression of endogenous alpha/beta T-cell receptor (TCR).

Methods of transfecting of cells are well known in the art, but highly efficient transfection methods such as electroporation may be employed. For example, nucleic acids or vectors of the invention expressing the TCR or CAR constructs may be introduced into cells using a nucleofection apparatus.

The cell population for recombinant TCR- or CAR-expressing T-cells may be enriched after transfection of the cells. For example, the cells expressing the recombinant TCR or CAR may be sorted from those which do not; (e.g., via FACS) by use of an antigen bound by the recombinant TCR or CAR or by use of a TCR or CAR-binding antibody. Alternatively, the enrichment step comprises depletion of the non-T-cells or depletion of cells that lack recombinant TCR or CAR expression. For example, CD56+ cells can be depleted from a culture population.

The population of transgenic, recombinant TCR- or CAR-expressing cells may be cultured ex vivo in a medium that selectively enhances proliferation of CAR-expressing T-cells. Therefore, the recombinant TCR- or CAR-expressing T cell may be expanded ex vivo, for example in the presence of CD3 and/or CD28 agonists, e.g. agonist antibodies.

A sample of recombinant TCR- or CAR-expressing T-cells may be preserved (or maintained in culture). For example, a sample may be cryopreserved for later expansion or analysis.

Recombinant TCR- or CAR-expressing T cells may be employed in combination with other therapeutics, for example checkpoint inhibitors including PD-L1 antagonists, e.g. anti PD1 antibody or anti PDL1 antibody.

In an embodiment, there is provided a T-cell, or a population of T-cells, that has been engineered to express any of the above antigen-binding polypeptides (e.g. a recombinant TCR or CAR) on its surface. Suitably, the T-cell is a cytotoxic T-cell.

In an embodiment, there is provided a T-cell, or a population of T-cells, engineered to express any of the above antigen-binding polypeptides (e.g. a recombinant TCR or CAR) on its surface, for use in medicine

The invention provides a pharmaceutical composition comprising a T-cell of the invention, or a population of T-cells (for example engineered to express any of the above antigen-binding polypeptides, e.g. a recombinant TCR or CAR on its surface).

The T-cell may for example be a helper (CD4+) or cytotoxic (CD8+) T-cell. The population of T-cells may be a mixed population of CD4+ and CD8+ T cells. Suitably the T-cell is a cytotoxic T-cell.

There is provided a method of treating a human patient suffering from cancer wherein the cells of the cancer express a sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof, or of preventing a human from suffering from cancer which cancer would express a sequence selected from any of SEQ ID NOs. 1 to 17 and 57 and variants and fragments, such as immunogenic fragments, thereof described herein, (for example a sequence comprising or consisting of the sequence selected from any one of SEQ ID NOs. 18 to 56 and 58 and a variant sequence thereof), which method comprises administering to said human a T-cell or population of T-cells of the invention.

In an embodiment the T-cell or population of T-cells of the invention is for use in treating or preventing cancer in a human, wherein the cells of the cancer express a corresponding sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof.

### Combination Therapies

Methods of treating cancer according to the invention may be performed in combination with other therapies, especially checkpoint inhibitors and interferons.

According to the invention the polypeptides, nucleic acids, vectors, exosome, antigen-binding polypeptide and adoptive cell therapies (APC and T cell-based) can be used in combination with other components designed to enhance their immunogenicity, for example, to improve the magnitude and/or breadth of the elicited immune response, or provide other activities (e.g., activation of other aspects of the innate or adaptive immune response, or destruction of tumor cells).

Accordingly, the invention provides a composition of the invention (i.e. an immunogenic, vaccine or pharmaceutical composition) or a kit of several such compositions comprising a polypeptide, nucleic acid or vector of the invention together with a pharmaceutically acceptable carrier; and (i) one or more further immunogenic or immunostimulant polypeptides (e.g., interferons, IL-12, checkpoint blockade molecules or nucleic acids encoding such, or vectors comprising such nucleic acids) or, (ii) small molecules (e.g., HDAC inhibitors or other drugs that modify the epigenetic profile of cancer cells) or biologicals (delivered as polypeptides or nucleic acids encoding such, or vectors comprising such nucleic acids) that will enhance the translation and/or presentation of the polypeptide products that are the subject of this invention.

Checkpoint inhibitors, which block normal proteins on cancer cells, or the proteins on the T cells that respond to them, may be a particularly important class of drugs to combine with CST-antigen based therapies, since these inhibitors seek to overcome one of cancer's main defences against an immune system attack.

Thus, an aspect of the invention includes administering a polypeptide, nucleic acid, vector, exosome, antigen-binding polypeptide, composition, immunoresponsive cell, immunoresponsive cell population, T-cell, T-cell population, or antigen presenting cell of the present invention in combination with a checkpoint inhibitor. Example check point inhibitors are selected from PD-1 inhibitors, such as pembrolizumab, (Keytruda) and nivolumab (Opdivo), PD-L1 inhibitors, such as atezolizumab (Tecentriq), avelumab (Bavencio) and durvalumab (Imfinzi) and CTLA-4 inhibitors such as ipilimumab (Yervoy).

Interferons (e.g., alpha, beta and gamma) are a family of proteins the body makes in very small amounts. Interferons may slow down or stop the cancer cells dividing, reduce the ability of the cancer cells to protect themselves from the immune system and/or enhance multiple aspects of the adaptive immune system. Interferons are typically administered as a subcutaneous injection in, for example the thigh or abdomen.

Thus, an aspect of the invention includes administering a polypeptide, nucleic acid, vector, antigen-binding polypeptide vector, exosome, antigen-binding polypeptide, composition, immunoresponsive cell, immunoresponsive cell population, T-cell, T-cell population, or antigen presenting cell, or composition of the present invention in combination with interferon e.g. selected from any one or more of interferon alpha, gamma, beta, or lambda and/or an interleukin (e.g. selected from any one or more of IL1, 2, 4, 6, 7, 15, 17, and 23).

Different modes of the invention may also be combined, for example polypeptides, nucleic acids, vectors and antigen-binding polypeptides of the invention may be combined with an APC, a immunoresponsive cell, immunoresponsive cell population, T-cell or a T-cell population of the invention (discussed infra).

One or more modes of the invention may also be combined with conventional anti-cancer chemotherapy and/or radiation.

The present invention additionally provides, a polypeptide, nucleic acid, vector, exosome, antigen-binding polypeptide, composition, immunoresponsive cell, immunoresponsive cell population, T-cell, T-cell population, or antigen presenting cell of the present invention in combination with one or more further therapeutic agent. Preferably the further therapeutic agent is a therapeutic agent designed to enhance their immunogenicity or to improve the magnitude and/or breadth of the elicited immune response, for example selected from one or more of; an anti-cancer antibody, small molecule anti-cancer therapeutic, a checkpoint inhibitor, interleukin or interferon, alternatively the further therapeutic agent may be selected from one or more polypeptide, fusion protein, nucleic acid, vector, exosome, antigen-binding polypeptide, composition, immunoresponsive cell, immunoresponsive cell population, T-cell, T-cell population, or antigen presenting cell of the present invention and/or other antigenic polypeptides (or polynucleotides or vectors encoding them), preferably which cause an immune response to be raised against cancer, preferably any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

Accordingly the present invention provides, a polypeptide, nucleic acid, vector, exosome, antigen-binding polypeptide, composition, immunoresponsive cell, immunoresponsive cell population, T-cell, T-cell population, or antigen presenting cell of the present invention for use in raising an immune response against cancer or tumour or for the treatment of cancer or tumour wherein said polypeptide, nucleic acid, vector, exosome, antigen-binding polypeptide, composition, immunoresponsive cell, immunoresponsive cell population, T-cell, T-cell population, or antigen presenting cell is for use or used or is for administration or is administered in combination with one or more further therapeutic agent, optionally administered or for administration separately, sequentially or simultaneously therewith. According to the invention the cancer or tumour may be any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

### Diagnostics

In another aspect, the invention provides methods for using one or more of the polypeptides or nucleic acids of the invention to diagnose cancer, or to diagnose human subjects suitable for treatment by polypeptides, nucleic acids, vectors, antigen-binding polypeptides, adoptive cell therapies, exosomes or compositions of the invention.

Thus the invention provides a method of diagnosing that a human is suffering from cancer, comprising the steps of: determining if the cells of said cancer express a polypeptide sequence selected from any of SEQ ID NOs. 1 to 17 and 57 and variants and fragments, such as immunogenic fragments, thereof e.g. selected from the sequences comprising or consisting of the sequence selected from any one of SEQ ID NOs. 18 to 56 and 58 and a variant sequence thereof; or a nucleic acid encoding said polypeptide sequence, and diagnosing said human as suffering from cancer if said polypeptide or corresponding nucleic acid or. nucleic acid encoding said polypeptide is overexpressed in said cancer cells.

The invention provides a method of diagnosing that a human is suffering from cancer which is for example any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC, comprising the steps of: determining if the cells of said cancer express a polypeptide sequence selected from any one of any of SEQ ID NOs. 1 to 17 and 57 and variants and fragments, such as immunogenic fragments thereof, for example a sequence comprising or consisting of a sequence selected from any one of SEQ ID NOs. 18 to 56 and 58 and a variant sequence thereof; or a nucleic acid encoding said polypeptide sequence, and diagnosing said human as suffering from cancer which is for example any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC, if said polypeptide or corresponding nucleic acid or. nucleic acid encoding said polypeptide is overexpressed in said cancer cells.

As used herein, “overexpressed” in cancer cells means that the level of expression in cancer cells is higher than in normal cells.

The overexpression can be determined by reference to the level of the nucleic acid or polypeptide of the invention in a control human subject known not to have the cancer. Thus overexpression indicates that the nucleic acid or polypeptide of the invention is detected at a significantly higher level (e.g., a level which is any of between 5 and 500%, 10 and 250%, 20 and 100%, 30 and 50% 30%, 50%, 100% or 500% higher) in the test subject than in the control subject. In case the control human subject has an undetectable level of the nucleic acid or polypeptide of the invention, then the diagnosis can be arrived at by detecting the nucleic acid or polypeptide of the invention.

The invention also provides a method of treating a human suffering from cancer, comprising the steps of:
(a) determining if the cells of said cancer express a polypeptide sequence selected from any of SEQ ID NOs. 1 to 17 and 57 and variants and fragments, such as immunogenic fragments, thereof (for example a sequence comprising or consisting of the sequence selected from any one of SEQ ID Nos. 18 to 56 and 58 and a variant sequence thereof) or a nucleic acid encoding said polypeptide, optionally in a biological sample obtained from said human; and if so (b) administering to said human a corresponding polypeptide, nucleic acid, vector, composition, immunoresponsive cell, immunoresponsive cell population, T-cell population, T-cell, antigen presenting cell or antigen-binding polypeptide or combination of the invention.

There is also provided use of a polypeptide comprising a sequence selected from:
(a) the sequence of any of SEQ ID NOs. 1 to 17 and 57;
(b) a variant of the sequence of (a); and
   a fragment, such as an immunogenic fragment, of the sequences of (a) and (b), (for example a sequence comprising or consisting of the sequence selected from any one of SEQ ID NOs. 18 to 56 and 58 and a variant sequence thereof), isolated from the tumor of a human suffering from cancer, or use of a nucleic acid encoding said polypeptide, as a biomarker for the determination of whether said human would be suitable for treatment by a vaccine comprising a corresponding polypeptide, nucleic acid, vector, composition, immunoresponsive cell, immunoresponsive cell population, T-cell population, T-cell, antigen presenting cell or antigen-binding polypeptide or combination of the invention.

Suitably, the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

Suitably the polypeptide of the invention has a sequence selected from any of SEQ ID NOs. 1 to 17 and 57 or a fragment, such as an immunogenic fragment, thereof (for example a sequence comprising or consisting of the sequence selected from any one of SEQ ID NOs. 18 to 56 and 58 and a variant sequence thereof).

Suitably the nucleic acid of the invention has or comprises a sequence encoding an amino acid sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof.

Kits for detecting the presence of nucleic acids are well known. For example, kits comprising at least two oligonucleotides which hybridise to a polynucleotide may be used within a real-time PCR (RT-PCR) reaction to allow the detection and semi-quantification of specific nucleic acids. Such kits may allow the detection of PCR products by the generation of a fluorescent signal as a result of Forster Resonance Energy Transfer (FRET) (for example TaqMan® kits), or upon binding of double stranded DNA (for example, SYBR® Green kits). Some kits (for example, those containing TaqMan® probes that span the exons of the target DNA) allow the detection and quantification of mRNA, for example transcripts encoding nucleic acids of the invention. Assays using certain kits may be set up in a multiplex format to detect multiple nucleic acids simultaneously within a reaction. Kits for the detection of active DNA (namely DNA that carries specific epigenetic signatures indicative of expression) may also be used. Additional components that may be present within such kits include a diagnostic reagent or reporter to facilitate the detection of a nucleic acid of the invention.

Nucleic acids of the invention may also be detected via liquid biopsy, using a sample of blood from a patient. Such a procedure provides a non-invasive alternative to surgical biopsies. Plasma from such blood samples may be isolated and analysed for the presence of nucleic acids of the invention.

Polypeptides of the invention may be detected by means of antigen-specific antibodies in an ELISA type assay to detect polypeptides of the invention in homogenized preparations of patient tumor samples. Alternatively, polypeptides of the invention may be detected by means of immunohistochemical analyses, which identify the presence of the polypeptide antigens by using light microscopy to inspect sections of patient tumor samples that have been stained by using appropriately labeled antibody preparations. As a further alternative, polypeptides of the invention may be detected by means of immunohistochemical analyses, which identify the presence of the polypeptide antigens by using light microscopy to inspect sections of patient tumor samples that have been stained by using appropriately labeled antibody preparations.

Polypeptides of the invention may also be detected by determining whether they are capable of stimulating T-cells raised against the said polypeptide. The invention provides a method of treatment of cancer, for example any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC, in a human which comprises (i) detecting the presence of a nucleic acid or polypeptide according to the invention, optionally in a biological sample obtained from said human and (ii) administering to the subject a nucleic acid, polypeptide, vector, cell, antigen-binding polypeptide, immunoresponsive cell, immunoresponsive cell population, T-cell or T-cell population or composition or combination according to the invention (and preferably administering the same nucleic acid or polypeptide or fragment thereof that has been detected).

The invention provides a method of treatment of cancer, for example any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC, in a human which comprises administering to the subject a nucleic acid, polypeptide, vector, antigen-binding polypeptide, cell, T-cell or T-cell population or composition or combination according to the invention, in which subject the presence of a (and preferably the same) nucleic acid or polypeptide according to the invention has been detected, optionally in a biological sample obtained from said human.

In particular, the cancer to be diagnosed and if appropriate treated is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC).

Where a polypeptide of the invention of any of SEQ ID Nos. 1 to 17 and 57 or a fragment thereof, for example a sequence comprising or consisting of the sequence selected from any one of SEQ ID NOs. 18 to 56 and 58 and a variant sequence thereof, is detected then the cancer might be cancer, for example any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

### Specific Embodiments

In an embodiment, the CST antigen polypeptide comprises or consists of any of SEQ ID NOs. 1 to 17 and 57 and variant thereof and a fragment thereof, such as an immunogenic fragment. Exemplary fragments comprise or consist of a sequence comprising or consisting of the sequence selected from any one of SEQ ID NOs. 18 to 56 and 58 and a variant sequence thereof. Exemplary nucleic acids comprise those encoding a sequence selected from any of SEQ ID NOs. 1 to 17 and 57 and variants and fragments, such as immunogenic fragments, thereof, for example a sequence comprising or consisting of the sequence selected from any one of SEQ ID NOs. 18 to 56 and 58 and a variant sequence thereof. Corresponding nucleic acids (e.g., DNA or RNA), T-cells, T-cell populations, cytotoxic cells, antigen-binding polypeptides, antigen presenting cells and exosomes as described supra are provided. Said nucleic acids (e.g., DNA or RNA), T-cells, T-cell populations, antigen-binding polypeptides, antigen presenting cells and exosomes may be used in the treatment of cancer, especially any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC. Related methods of diagnosis are also provided.

Further embodiments of the invention are defined by the following clauses:
1. An isolated polypeptide comprising a sequence selected from any of:
(a) the sequence of any one of SEQ ID NOs. 1 to 17 and 57; and
(b) a variant of the sequences of (a); and
(c) a fragment of the sequences of (a) or (b); optionally wherein said fragment and/or variant is an immunogenic fragment and/or immunogenic variant.
2. An isolated polypeptide according to clause 1 comprising or consisting of the sequence selected from any one of SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC.
3. The isolated polypeptide according to clause 1 or 2, wherein the polypeptide has the ability to bind to or binds to an MHC molecule, preferable either MHC class 1 or class 2 molecule, and/or wherein said polypeptide, when bound to said MHC, is immunogenic and/or capable of being recognized by (binding to) CD4 and/or CD8 T cells.
4 The isolated polypeptide according to any one of clauses 1 to 3, wherein the polypeptide or fragment or variant has an overall length of between 7 to 30 amino acids, optionally between any of 8 and 9, 8 and 10, 8 and 11, 8 and 12, 8 and 13, 8 and 14, 8 and 15, 8 and 16, 8 and 17, 8 and 18, 8 and 19, or 8 and 20 contiguous amino acids.
5 The isolated polypeptide according to any one of clauses 1 to 4, wherein the polypeptide includes non-peptide bonds.
6 The isolated polypeptide according to any one of clauses 1 to 5, wherein the polypeptide, is part of a fusion protein.
7. The isolated polypeptide according to clause 6 wherein the polypeptide is fused to a second or further polypeptides selected from (i) one or more other polypeptides according to clause 1 to 5, (ii) one or more other polypeptides which are cancer associated antigens, optionally esophageal cancer associated antigens, (iii) one or more polypeptide sequences which are capable of enhancing an immune response (i.e. immunostimulant sequences) and (iv) one or more polypeptide sequences, e.g. comprising universal CD4 helper epitopes, which are capable of providing strong CD4+ help to increase CD8+ T-cell responses to antigen epitopes.
8. The isolated polypeptide according to clause 6 or 7 which is a fusion protein which comprises two or more, preferably any of two, three, four or five, six, seven, eight, nine, ten, eleven or twelve sequences selected from sequences selected from the polypeptides according to clause 1 to 5, optionally wherein said polypeptide sequences are all different.
9. An isolated nucleic acid encoding the polypeptide according to any one of clauses 1 to 8.
10. The nucleic acid according to clause 9 which is a DNA.
11. The nucleic acid according to clause 9 or 10 which is codon optimised for expression in a human host cell.
12. The nucleic acid according to clause 9 which is an RNA.
13. The nucleic acid according to any one of clauses 9 to 12 which is an artificial nucleic acid sequence.
14. A vector comprising the nucleic acid according to any one of clauses 9 to 13.
15. The vector according to clause 14 which comprises DNA encoding regulatory elements suitable for permitting transcription of a translationally active RNA molecule in a human host cell.
16. The vector according to clause 14 or clause 15 which is a viral vector.
17. The vector according to clause 16 which is an adenoviral vector, an adeno-associated virus (AAV), alphavirus, herpes virus, arena virus, measles virus, poxvirus, paramyxovirus, lentivirus and rhabdovirus vector.
18. A pharmaceutical composition comprising a polypeptide, nucleic acid or vector according to any one of clauses 1 to 17 together with a pharmaceutically acceptable carrier, preferably wherein said pharmaceutical composition is an immunogenic pharmaceutical composition.
19. A vaccine composition comprising a polypeptide, nucleic acid or vector according to any one of clauses 1 to 17 together with a pharmaceutically acceptable carrier.
20. The composition according to clause 18 or clause 19 which comprises one or more immunostimulants.
21. The composition according to clause 20 wherein the one or more immunostimulants are selected from aluminium salts, saponins, immunostimulatory oligonucleotides, oil-in-water emulsions, aminoalkyl glucosaminide 4-phosphates, lipopolysaccharides and derivatives thereof and other TLR4 ligands, TLR7 ligands, TLR8 ligands and TLR9 ligands, IL12, interferons, incomplete freund's adjuvant (IFA), aluminum-based adjuvants, TLR agonists, synthetic double-stranded RNAs (dsRNAs), glucopyranosyl lipid a (GLA), imidazoquinolines, CPG oligodeoxynucleotides (ODNs), cyclic dinucleotides (CDNs), manganese-based adjuvants; metabolic adjuvants, nanoparticle-based adjuvant, water-in-oil nanoemulsions, micro or nanoparticle adjuvants, CGAS-STING/STING agonists, cytokines, self-assembling peptides, virus-like particles (VLPs), inorganic nanoparticles, caged protein nanoparticles, nucleoside-unmodified mRNA, PRR ligands (pattern recognition receptor ligands), mRNA encoding functional proteins, component lipid-based adjuvants, peptide and glycolipid immunostimulants, TLR agonists.
22. The composition according to any one of clauses 18 to 21 which is a sterile composition suitable for parenteral administration.
23. A polypeptide, nucleic acid, vector or composition according to any one of clauses 1 to 22 for use in medicine.
24. A method of raising an immune response in a human which comprises administering to said human the polypeptide, nucleic acid, vector or composition according to any one of clauses 1 to 22.
25. The method according to clause 24 wherein the immune response is raised against a cancerous tumour expressing, and or presenting on its surface, a sequence selected from (a) SEQ ID NOs. 1 to 17 and 57 and variants and immunogenic fragments of any one thereof, (b) SEQ ID NOs. 18 to 56 and 58; and a variant sequence thereof, optionally wherein said variant sequence is immunogenic and/or maintains capacity to bind to MHC molecule(s) and/or induce T cells cross-reacting with said polypeptide and/or variant, optionally when bound to MHC. 26. A polypeptide, nucleic acid, vector or composition according to any one of clauses 1 to 22 for use in raising an immune response in a human.
27. The polypeptide, nucleic acid, vector or composition for use according to clause 26 wherein the immune response is raised against a cancerous tumour expressing a corresponding sequence selected from (a) SEQ ID NOs. 1 to 17 and 57 and immunogenic fragments or variants of any one thereof, (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC.
28. A method of treating a human patient suffering from cancer wherein the cells of the cancer express a sequence selected from (a) SEQ ID NOs. 1 to 17 and 57 and immunogenic fragments and variants of any one thereof, (b) SEQ ID NOs. 18 to 56 and 58 and a variant sequence thereof, optionally wherein said variant sequence thereof maintains capacity to bind to MHC molecule(s) and/or induce T cells cross-reacting with said variant, or of preventing a human from suffering from cancer which cancer would express a sequence selected from (a) SEQ ID NOs. 1 to 17 and 57 and immunogenic fragments and variants of any one thereof, (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC, which method comprises administering to said human a corresponding polypeptide, nucleic acid, vector or composition according to any one of clauses 1 to 22.
29. A polypeptide, nucleic acid, vector or composition according to any one of clauses 1 to 22 for use in treating or preventing cancer in a human, wherein the cells of the cancer express a corresponding sequence selected from (a) SEQ ID NOs. 1 to 17 and 57 and immunogenic fragments of any one thereof, (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC.
30. A polypeptide, nucleic acid, vector or composition according to any one of clauses 1-22 for use in the ex vivo stimulation and/or amplification of T-cells derived from a human suffering from cancer, for subsequent reintroduction of said stimulated and/or amplified T-cells into the said human for the treatment of the said cancer in the said human.
31. A method of treatment of cancer in a human, wherein the cells of the cancer express a sequence selected from (a) SEQ ID NOs. 1 to 17 and 57 and immunogenic fragments and variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC, which comprises taking from said human a population of white blood cells comprising at least T-cells optionally with antigen-presenting cells, stimulating and/or amplifying said T-cells in the presence of a corresponding polypeptide, nucleic acid, vector or composition according to any one of clauses 1 to 22, and reintroducing some or all of said white blood cells at least stimulated and/or amplified T-cells into the human.
32. A method or a polypeptide, nucleic acid, vector or composition for use according to any one of clauses 23 to 31 wherein the cancer is selected from any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.
33. A process for preparing a T-cell population which is cytotoxic for cancer cells which express a sequence selected from (a) SEQ ID NOs. 1 to 17 and 57 and immunogenic fragments and variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC; which comprises (i) obtaining T-cells optionally with antigen-presenting cells from a cancer patient and (ii) stimulating and amplifying the T-cell population ex vivo with a corresponding polypeptide, nucleic acid, vector or composition according to any one of clauses 1 to 22.
34. A T-cell population obtainable by the process of clause 33, preferably wherein said T-cell is immunoresponsive to said polypeptide, nucleic acid, vector or composition.
35. A T-cell which has been stimulated with a polypeptide, nucleic acid, vector or composition according to any one of clauses 1 to 22, preferably wherein said T-cell is T-cell is immunoresponsive to the polypeptide, nucleic acid, vector or composition.
36. An antigen presenting cell modified by ex vivo loading with the polypeptide, nucleic acid, vector or composition according to any one of clauses 1 to 22 or genetically engineered to express the polypeptide according to any one of clauses 1 to 8.
37. The antigen presenting cell of clause 36 which is a dendritic cell.
38. An exosome loaded with a polypeptide prepared from cells loaded with a polypeptide, nucleic acid, vector or composition according to any one of clauses 1 to 22 or genetically engineered to express the polypeptide according to any one of clauses 1 to 8.
39. A pharmaceutical composition comprising the T-cell population, the T-cell, antigen presenting cell or exosome according to any one of clauses 34 to 38 together with a pharmaceutically acceptable carrier.
40. A T-cell population, T-cell, antigen presenting cell, exosome or composition according to any one of clauses 34 to 39 for use in medicine.
41. A method of treating a human suffering from cancer wherein the cells of the cancer express a sequence selected from (a) SEQ ID NOs. 1-17 and 57 and immunogenic fragments and variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC, or of preventing a human from suffering from cancer wherein the cells of the cancer would express a sequence selected from (a) SEQ ID NOs. 1 to 17 and 57 and immunogenic fragments and variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC; which comprises administering to said human the T-cell population, the T-cell, antigen presenting cell, exosome or composition according to any one of clauses 34 to 39.
42. A T-cell population, T-cell, antigen presenting cell, exosome or composition according to any one of clauses 34 to 39 for use in treating or preventing cancer in a human, wherein the cells of the cancer express a corresponding sequence selected from (a) SEQ ID NOs. 1-17 and 57 and immunogenic fragments of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC.
43. A process, a method or a T-cell population, T-cell, antigen presenting cell, exosome or composition for use according to any one of clauses 33, 41 and 42 wherein the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.
44. An isolated antigen-binding polypeptide which is immunospecific for the polypeptide according to any one of clauses 1 to 8, optionally when said polypeptide is bound to an MHC molecule.
45. The antigen-binding polypeptide according to clause 44 which is a monoclonal antibody or a fragment thereof or T cell receptor or fragment thereof, optionally wherein either said fragment is an antigen binding fragment.
46. The antigen-binding polypeptide according to clause 44 or clause 45 which is coupled to a cytotoxic moiety.
47. A pharmaceutical composition comprising the antigen-binding polypeptide according to any one of clauses 44 to 46 together with a pharmaceutically acceptable carrier.
48. An antigen-binding polypeptide according to any one of clauses 44 to 46 or pharmaceutical composition according to clause 47, for use in medicine.
49. A method of treating a human suffering from cancer wherein the cells of the cancer express a sequence selected from (a) SEQ ID NOs. 1 to 17 and 57 and immunogenic fragments and variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC, or of preventing a human from suffering from cancer wherein the cells of the cancer would express a sequence selected from (a) SEQ ID NOs. 1 to 17 and 57 and immunogenic fragments and variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC, which comprises administering to said human the antigen-binding polypeptide or composition according to any one of clauses 44 to 46 and 47.
50. An antigen-binding polypeptide or a composition according to any one of clauses 44 to 46 and 47 for use in treating or preventing cancer in a human, wherein the cells of the cancer express a corresponding sequence selected from (a) SEQ ID NOs.1 to 17 and 57 and immunogenic fragments of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC.
51. A method, antigen-binding polypeptide or composition for use according to clause 49 or clause 50 wherein the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.
52. An isolated antigen-binding polypeptide which is immunospecific for and/or binds to an HLA-bound polypeptide that is or is part of the polypeptide according to any one of clauses 1 to 8.
53. The antigen-binding polypeptide according to clause 52 which is a T-cell receptor derived molecule or T-cell receptor or a fragment thereof, preferably an antigen binding fragment thereof or which is an antibody mimic of any thereof.
54. The antigen-binding polypeptide according to clause 52 or clause 53 which is coupled to another polypeptide that is capable of binding to an immunoresponsive cell or T-cell or cytotoxic cell or other immune components in a subject, preferably that is capable of binding to a T-cell, optionally wherein said polypeptide is an antibody derived molecule.
55. A cell that has been engineered to express the antigen-binding polypeptide of any one of clauses 52 to 54 on its surface, alternatively to secrete said antigen-binding polypeptide as a soluble polypeptide or molecule.
56. The cell according to clause 55 which is an immune cell (e.g. immunoresponsive cell) and/or cytotoxic cell and/or T-cell.
57. A pharmaceutical composition comprising the antigen binding protein of any of clauses 52 to 54 or a cell according to clause 55 or clause 56.
58. A cell according to clause 55 or clause 56, an antigen binding protein of any of clauses 52 to 54, or a pharmaceutical composition according to clause 57 for use in medicine.
59. A method of treating a human patient suffering from cancer wherein the cells of the cancer express a sequence selected from (a) SEQ ID NOs. 1 to 17 and 57 and immunogenic fragments and variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC, or of preventing a human from suffering from cancer which cancer would express a sequence selected from (a) SEQ ID NOs. 1 to 17 and 57 and immunogenic fragments and variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC, which method comprises administering to said an antigen binding polypeptide according to any of clauses 52 to 54 or a cell according to clause 55 or clause 56 or a pharmaceutical composition according to clause 57, optionally wherein said cell is a human cell.
60. An antigen binding polypeptide according to any of clauses 52 to 54 or a cell according to clause 55 or clause 56, or a pharmaceutical composition according to clause 57, for use in treating or preventing cancer in a human, wherein the cells of the cancer express a corresponding sequence selected from (a) SEQ ID NOs. 1 to 17 and 57 and immunogenic fragments of any one thereof (b) SEQ ID NOs. 18 to 56 and 58 and a variant sequence thereof optionally wherein said variant sequence is immunogenic and/or maintains capacity to bind to MHC molecule(s) and/or induce T cells cross-reacting with said polypeptide and/or variant, optionally when bound to MHC.
61. A method of diagnosing a human as suffering from cancer, comprising the steps of: determining if the cells of said cancer express a polypeptide sequence selected from (a) SEQ ID NOs. 1 to 17 and 57 and immunogenic fragments or variants of any one thereof (b) SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC, or a nucleic acid encoding said polypeptide sequence, and diagnosing said human as suffering from cancer if said polypeptide or encoding nucleic acid is overexpressed in said cancer cells.
62. A method of diagnosing that a human as suffering from cancer which is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC, comprising the steps of: determining if the cells of said cancer express the polypeptide sequence selected from any of SEQ ID NOs. 1 to 17 and 57 or 18 to 56 and 58, and fragments or immunogenic fragments and/or variants or immunogenic variants thereof; optionally wherein said variant sequence is immunogenic and/or maintains capacity to bind to MHC molecule(s) and/or induce T cells cross-reacting with said polypeptide and/or variant, optionally when bound to MHC, or a nucleic acid encoding said polypeptide sequence, and diagnosing said human as suffering from cancer which is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC if said polypeptide or corresponding nucleic acid is overexpressed in said cancer cells.
63. A method of diagnosing that a human as suffering from cancer which is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC, comprising the steps of: determining if the cells of said cancer express a polypeptide sequence selected from any of SEQ ID NOs. 1 to 17 and 57 or 18 to 56 and 58, and fragments or immunogenic fragments and/or variants or immunogenic variants thereof; optionally wherein said variant sequence is immunogenic and/or maintains capacity to bind to MHC molecule(s) and/or induce T cells cross-reacting with said polypeptide and/or variant, optionally when bound to MHC, or a nucleic acid encoding said polypeptide sequence, and diagnosing said human as suffering from cancer which is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC, if corresponding nucleic acid is overexpressed in said cancer cells.
64. A method of treating a human suffering from cancer, comprising the steps of:
(a) determining if the cells of said cancer express a polypeptide sequence selected from any of SEQ ID NOs. 1 to 17 and 57 or 18 to 56 and 58, and fragments or immunogenic fragments and/or variants or immunogenic variants thereof; optionally wherein said variant sequence is immunogenic and/or maintains capacity to bind to MHC molecule(s) and/or induce T cells cross-reacting with said polypeptide and/or variant, optionally when bound to MHC, or a nucleic acid encoding said polypeptide sequence; and if so:
(b) administering to said human a corresponding polypeptide, nucleic acid, vector, composition, T-cell population, T-cell, antigen presenting cell, exosome, antigen-binding polypeptide or cell according to any one of clauses 1 to 22, 34 to 39, 44 to 47, and 52 to 57.
65. Use of a polypeptide comprising a sequence selected from:
(a) the sequence of any one of SEQ ID NOs. 1-17 and 57; or
(b) a variant or immunogenic variant of the sequences of (a); and
(c) a fragment or immunogenic fragment of the sequences of (a) or (b), for example selected from any of SEQ ID NOs. 18 to 56 and 58 and a variant sequence thereof, optionally wherein said variant sequence is immunogenic and/or maintains capacity to bind to MHC molecule(s) and/or induce T cells cross-reacting with said polypeptide and/or variant, optionally when bound to MHC; isolated from the tumour of a human suffering from cancer, or use of a nucleic acid encoding said polypeptide, as a biomarker for the determination of whether said human would be suitable for treatment by a vaccine comprising a corresponding polypeptide, nucleic acid, vector, composition, T-cell population, T-cell, antigen presenting cell, exosome, antigen-binding polypeptide or cell according to any one of clauses 1 to 22, 34 to 39, 44 to 47, and 52 to 57.
66. The method or use according to any of, clause 59, clause 60, clause 61, clause 64 or clause 65 wherein the cancer is any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, particularly esophageal adenocarcinoma (EAC) or esophageal squamous cell carcinoma (ESSC).
67. A method or a polypeptide, nucleic acid, vector or composition for use according to any one of clauses 25 and 27 to 31, wherein the polypeptide comprises a sequence selected from:
(a) the sequence of any one of SEQ ID NOs. 1 to 17 and 57; and
(b) a variant or immunogenic variant of the sequences of (a); and
(c) a fragment or immunogenic fragment of the sequences of (a) or (b), optionally wherein the polypeptide comprises or consists of a sequence selected from any one of SEQ ID NOs. 18 to 56 and 58 and/or optionally wherein the nucleic acid comprises or consists of a sequence encoding a sequence of any one of SEQ ID NOs. 1 to 58; and wherein the cancer is selected from any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC
68. A method according to clause 49 or 59 or an antigen-binding polypeptide, cell or composition for use according to clause 50 or 60 wherein the polypeptide comprises a sequence selected from:
(a) the sequence of any one of SEQ ID NOs. 1 to 17 and 57; and
(b) a variant or immunogenic variant of the sequences of (a); and
(c) a fragment or immunogenic fragment of the sequences of (a) or (b), optionally wherein the polypeptide comprises or consists of a sequence selected from any one of SEQ ID NOs. 18 to 56 and 58 and/or optionally wherein the nucleic acid comprises or consists of a sequence encoding a sequence of any one of SEQ ID NOs. 1 to 58; and wherein the cancer is selected from any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.
69. A process, a method or a T-cell population, T-cell, antigen presenting cell, exosome or composition for use according to any one of clauses 33, 41 and 42 wherein the polypeptide comprises a sequence selected from:
(a) the sequence of any one of SEQ ID NOs. 1 to 17 and 57; and
(b) a variant or immunogenic variant of the sequences of (a); and
(c) a fragment or immunogenic fragment of the sequences of (a) or (b), optionally wherein the polypeptide comprises or consists of a sequence selected from any one of SEQ ID NOs. 18 to 56 and 58 and/or optionally wherein the nucleic acid comprises or consists of a sequence encoding a sequence of any one of SEQ ID NOs. 1 to 58, and wherein the cancer is selected from any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.
70. The method or use according to clause 64 or clause 65 wherein the polypeptide comprises a sequence selected from:
(a) the sequence of any one of SEQ ID NOs. 1 to 17 and 57; and
(b) a variant or immunogenic variant of the sequences of (a); and
(c) a fragment or immunogenic fragment of the sequences of (a) or (b), optionally wherein the polypeptide comprises or consists of a sequence selected from any one of SEQ ID NOs. 18 to 56 and 58 and/or optionally wherein the nucleic acid comprises or consists of a sequence encoding a sequence of any one of SEQ ID NOs. 1 to 58; and wherein the cancer is selected from any of ovarian cancer, gastrointestinal cancer, pancreatic cancer, prostate cancer, stromal cancer, osteosarcoma, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC), particularly esophageal cancer, or EAC or ESSC.

### EXAMPLES

### Example 1—Cancer-Specific Transcript Identification

The objective was to identify novel transcripts specific to human Esophageal Adenocarcinoma (EAC)

High quality EAC RNAseq data were obtained from The Cancer Genome Atlas Consortium (TCGA) and used to generate transcriptome assemblies via a reference-guided approach. Potentially cancer-specific transcripts were identified through differential expression analysis and their expression distributions (measured as TPM) examined across 33 TCGA-defined indications and 48 anatomical sites for which RNAseq data was available (from GTEx, The Genotype-Tissue Expression Consortium, 2015, Science, 348:648-60) (FIGS. 1-17 and 26). Putative ORFs were inferred from these transcripts from all three reading frames starting with a conventional start codon (ATG), encoding a peptide equal to or longer than eight amino acids, and terminating either with a stop codon or by reaching the end of the transcript.

### Example 2—Immunopeptidomic Analysis

Mass spectrometry (MS)-based immunopeptidomics analysis is a powerful technology that allows for the direct detection of specific peptides associated with HLA molecules (HLAp) and presented on the cell surface. The technique consists of affinity purification of the HLAp from biological samples such as cells or tissues by anti-HLA antibody capture. The isolated HLA molecules and bound peptides are then separated from each other and the eluted peptides are analyzed by nano-ultra performance liquid chromatography coupled to mass spectrometry (nUPLC-MS) (Freudenmann et al., 2018, Immunology 154 (3): 331-345). In the mass spectrometer, specific peptides of defined charge-to-mass ratio (m/z) are selected, isolated, fragmented, and then subjected to a second round of mass spectrometry (MS/MS) to reveal the m/z of the resulting fragment ions. The fragmentation spectra (MS/MS) can then be interrogated to precisely identify the amino acid sequence of the selected peptide that gave rise to the detected fragment ions.

MS/MS spectral interpretation and subsequent peptide sequence identification relies on the match between experimental data and theoretical spectra created from peptide sequences found in a reference database. Although it is possible to search MS data by using pre-defined lists corresponding to all open reading frames (ORFs) derived from the known transcriptome or even the entire genome (Nesvizhskii et al., 2014, Nat. Methods 11:1114-1125), interrogating these very large sequence databases leads to very high false discovery rates (FDR) that limit the identification of presented peptides. Further technical issues (e.g., mass of leucine=mass of isoleucine), and theoretical issues (e.g., peptide splicing (Liepe, et al., 2016, Science 354(6310): 354-358)) increase the limitations associated with use of very large databases, such as those produced from the known transcriptome or entire genome. Thus, in practice, it is exceptionally difficult to perform accurate immunopeptidomics analyses to identify novel antigens without reference to a well-defined set of potential polypeptide sequences (Li, et al., 2016, BMC Genomics 17 (Suppl 13): 1031).

The inventors procured frozen tumor tissue from 60 ovarian carcinomas (OV), 40 Breast cancer, 5 Renal, 33 Skin, 11 esophageal adeno carcinomas (EAC), 5 esophageal squamous cell carcinoma (ESSC), and 66 colorectal adenocarcinoma (COAD) patients. Samples between 0.2-1 g were homogenized, the lysate was centrifugate at high speed and the cleared lysate was mixed with protein A (ProA) beads covalently linked to an anti-human HLA class I monoclonal antibody (BB7 and/or W6/32). The mixture was incubated in the case of W6/32 single antibody incubation: overnight at 4° C. to improve HLA Class I molecule binding to antibody (Ternette et al., 2018 Proteomics 18, 1700465). The HLA Class I-bound peptides were eluted from the antibody by using 10% acetic acid, and the peptides were then separated from other high molecular mass components using reversed-phase column chromatography (Ternette et al., 2018 supra). The purified, eluted peptides were subjected to nUPLC-MS, and specific peptides of defined charge-to-mass ratio (m/z) were selected within the mass spectrometer, isolated, fragmented, and subjected to a second round of mass spectrometry (MS/MS) to reveal the m/z of the resulting fragment ions (Ternette et al., 2018 supra), producing an MS/MS dataset corresponding to the immunopeptidome for each of these tumor samples.

By applying detailed knowledge of immunopeptidomics evaluation, the inventors interrogated the spectra of the HLA-Class I dataset for the 60 ovarian carcinomas (OV), 40 Breast cancer, 5 Renal, 33 Skin, 11 esophageal adeno carcinomas (EAC), 5 esophageal squamous cell carcinoma (ESSC), and 66 colorectal adenocarcinoma (COAD) patients prepared by the inventors with the cancer-specific transcript-derived ORFs of Example 1. Since the majority of HLA class I-bound peptides found in cells are derived from constitutively expressed proteins, the simultaneous interrogation of these databases with the UniProt proteome helps to ensure that assignments of our ORF sequences to MS/MS spectra are correct. Two mass spectrometry methods were used to acquire MS/MS spectra, data dependent acquisition (DDA) and data independent acquisition (DIA). DDA MS/MS spectra are interrogated using PEAKS software, which, assigns a probability value (−10 lg P) to each assignment of spectra to quantify the assignment. DIA MS/MS spectra were interrogated using DIA-NN (Data Independent Acquisition by Neural Networks), (Nature Methods. 2020 January; 17 (1): 41-44) against a database of peptide sequences within our putative cancer-specific ORF sequences that are predicted to bind to common HLA alleles by NetMHCpan 4.0. Only peptide identification with calculated global Q value less than 1% were accepted, confirmed peptides and associated Q-values are shown in Table 2 below.

The results of these studies identified individual peptides that were associated with the HLA Class I molecules immunoprecipitated from tumor samples 60 ovarian carcinomas (OV), 40 Breast cancer, 5 Renal, 33 Skin, 11 esophageal adeno carcinomas (EAC), 5 esophageal squamous cell carcinoma (ESSC), and 66 colorectal adenocarcinoma (COAD) in the inventors' dataset, that corresponded to the amino acid sequence of cancer-specific transcript-derived ORFs, and did not correspond to polypeptide sequences present within the known human proteome (UniProt and/or masDB).

Further manual review of the peptide spectra assigned by the PEAKS and DIA-NN software to these ORF sequences confirmed the assignment of spectra to peptides that were mapped to 18 ORFs (EVA001 to EVA018, SEQ ID Nos. 1-17 and 57), which were encoded by single cancer-specific transcripts. The ORFs were thus defined as CST antigens (SEQ ID NOs. 1-17 and 57) as set out in Table 3.

The detection of peptides associated with the HLA Class I molecules confirms, that the 18 ORFs from which they were derived, were first translated in different tumor types, processed through the HLA Class I pathway and finally presented to the immune system in a complex with HLA Class I molecules. Table 3 shows the properties of the peptides (SEQ ID Nos. 18 to 56 and 58) found in the CST antigens (SEQ ID NO. 1-17 and 57) and their presentation in patient derived tumour tissue. FIGS. 1 to 17 and 26 show the identification of cancer specific transcripts specific to the esophageal adenocarcinoma (EAC) cancer type through the use of de novo assembly, the transcripts per million (TPM) were estimated for all transcripts and expression within several cancer types from TGCA (FIGS. 1 to 17 and FIG. 26, part B) was compared with expression across healthy tissue samples from the GTEx (FIGS. 1 to 17 and FIG. 26, part A).

Representative MS/MS spectra for peptides SEQ ID NOs. 18 to 56 and 58 were determined to provide a MS/MS peptide fragment profile from the tumors MS/MS datasets by using the PEAKS software thereby permitting a rendering of the spectrum indicating the positions of the linear peptide sequences that have been mapped to the fragment ions by PEAKS and DIA-NN to the peptides disclosed in Table 3, these spectra contained numerous fragments that precisely matched the sequences of the peptides (SEQ ID NOs. 18 to 56 and 58) that were discovered in these analyses.

The peptides detected in association with HLA Class I molecules from Table 3 were further assessed to determine their predicted strength of binding to HLA Class I type A, B and C supertypes by using the NetMHCpan 4.0 prediction software (http://www.cbs.dtu.dk/services/NetMHCpan/). The results of these prediction studies show that the peptides are predicted to bind to at least one of the haplotypes expressed by the patient derived tissues (see Table 3). The fact that the detected peptides were predicted to bind to the standard set of HLA types provides additional validation around their detection. Moreover, peptides discovered in tumor samples from the inventors' dataset were predicted by NetMHCpan 4.0 to bind to one of the HLA types which were detected in the patient samples.

Taken together, the peptide data shown in Table 2 and Table 3, and FIGS. 1 to 17 and 26, supply strong support for the translation, processing, and presentation of the corresponding CST antigens in ovarian cancer, gastrointestinal cancer, pancreatic cancer, prostate cancer, stromal cancer, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, esophageal cancer, esophageal adenocarcinoma (EAC) and esophageal squamous cell carcinoma (ESSC) patients, particularly esophageal cancer, or EAC or ESSC patients.

To further confirm the cancer-specificity of these CST antigens, the inventors processed normal tissue samples including (normal lung, normal kidney, normal liver, normal spleen, normal head/neck tissue, and normal breast tissue) and prepared for immunopeptidomic analysis. The inventors interrogated the spectra of the HLA-Class I dataset from these normal tissue samples, searching for all possible peptide sequences derived from the polypeptide sequences of CST antigens 1-18, alongside all the polypeptides found in the human proteome (UniProt) using the Peaks™ software (X). No peptides derived from CST antigens EVA001-EVA018 (SEQ ID Nos. 1-17 and 57) were detected in the set of normal tissue samples providing additional evidence that the CST have cancer-specific expression. To provide further certainty of the assignment of tumor tissue-derived MS spectra to the peptide sequences that we discovered, peptides with these discovered sequences were synthesized and subjected to nUPLC-MS2 using the same conditions applied to the tumor samples in the original study. Comparison of the spectra for synthetic and endogenous (i.e., tumor) peptides were made and m/z (mass to charge ratio) values of detected ion fragments were determined for each fragment peak in these MS/MS spectra. This analysis revealed a precise alignment of fragments (tiny differences in the experimentally determined m/z values between tumor- and synthetic peptide-derived fragment ions being well within the m/z tolerances of <0.05 Daltons), confirming the veracity of the assignment of each of the tumor tissue-derived spectra to the CST-encoded peptides (i.e. EVA001-EVA018 derived peptides).

In summary: the identification of immunopeptidomic peptides derived from the predicted ORFs, demonstrates that the CST transcripts are translated into polypeptides (SEQ ID NOs. 1 to 17 and 57; referred to as CST antigens EVA001 to EVA018) in tumor tissue. This is then processed by the immune surveillance apparatus of the cells, and component peptides are loaded onto HLA Class I molecules, enabling the cell to be targeted for cytolysis by T cells that recognize the resulting peptide/HLA Class I complexes. Thus, the identified CST antigens and fragments from these CST antigens are expected to be useful in a variety of therapeutic modalities for the treatment of esophageal cancer in patients whose tumors express these antigens.

TABLE 2
DIA MS/MS spectra Q-value and global Q-value Statistics for identified
peptides

| | | Peptide | | |
|---|---|---|---|---|
| ORF EVA | Peptide | SEQ ID | | |
| number | Sequence | No. | Q.Value | Global.Q.Value |
| EVA001 | KRYNRIMHDEL | 18 | 0.002663904 | 0.007688568 |
| EVA002 | KLLKLNMGSL | 19 | 0.002562788 | 0.007421494 |
| EVA003 | HPKTDAALL | 20 | 0.000167785 | 0.000347485 |
| EVA004 | FPSSPFRTV | 22 | 0.001206496 | 0.003467764 |
| EVA004 | FRTVPHLLL | 23 | 0.001521573751 | 0.004907622933 |
| EVA005 | SPSPLKSQL | 24 | 0.005007283296 | 0.008293348365 |
| EVA006 | LKKKTEIEL | 25 | 0.001327434 | 0.000399404 |
| EVA007 | AVSLTILAV | 26 | 0.000555494 | 0.001593807 |
| EVA008 | KRSSPVNSV | 27 | 0.002039152 | 0.005853823 |
| EVA008 | KRSSPVNSVLSK | 28 | 0.0007738607237 | 0.002474145731 |
| EVA009 | GLIHITLL | 29 | 0.000608921 | 0.001817958 |
| EVA009 | HPDGLIHITL | 30 | 0.001348718768 | 0.004336758517 |
| EVA010 | RPRPGKGSL | 31 | 0.003264802 | 0.009624445 |
| EVA011 | SPTLAPEIL | 32 | 0.003189604 | 0.009443883 |
| EVA012 | SRDHGVSL | 33 | 9.36E−05 | 9.36E−05 |
| EVA012 | VSLGPSPQMV | 34 | 0.00164390495 | 0.005321099889 |
| EVA013 | VDSSLSLL | 35 | 0.002166261 | 0.006237746 |
| EVA014 | MASRKKMI | 36 | 0.001218769 | 0.003518302 |
| EVA015 | SLIKQRPPRK | 37 | 0.000359712 | 0.001023664 |
| EVA015 | RSLIKQRPPR | 38 | 0.00017322 | 0.00037408 |
| EVA015 | SLIKQRPPR | 39 | 0.00753153814 | 0.0007308896165 |
| EVA015 | DPLPNRSLI | 40 | 0.002722323 | 0.009066211 |
| EVA015 | RSLIKQRPPRK | 41 | 0.002643948188 | 0.008751260117 |
| EVA016 | ILPPTPLRL | 42 | 0.001479122 | 0.001235228 |
| EVA016 | GAPTLSFVVA | 43 | 0.001208167 | 0.003824285464 |
| EVA016 | GSFILGSLM | 44 | 0.002697235 | 0.008996002376 |
| EVA016 | LMLPSDASKLR | 45 | 0.001936108456 | 0.006200040691 |
| EVA016 | LRSGAPTDTL | 46 | 0.002823861549 | 0.009411123581 |
| EVA016 | RFTEGKSTPGPFR | 47 | 0.001275278 | 0.0040635271 |
| EVA016 | ILPPTPLRL | 48 | 0.000120163 | 8.89E−05 |
| EVA016 | KLKSTFGAEI | 49 | 0.001402918017 | 0.0004725318868 |
| EVA016 | SFILGSLML | 50 | 0.000989298 | 0.003123116214 |
| EVA016 | GAPTLSFVVA | 51 | 0.001208167 | 0.003824285464 |
| EVA016 | GSFILGSLM | 52 | 0.002697235439 | 0.003824285464 |
| EVA016 | TSGPLKSPSR | 53 | 0.001102156 | 0.003482080298 |
| EVA017 | VQAEVGIRF | 54 | 0.002227172 | 0.006412842 |
| EVA015 | DPLPNRSLI | 55 | 0.002722323 | 0.009066211 |
| EVA008 | KRSSPVNSVLSK | 56 | 0.0007738607237 | 0.002474145731 |
| EVA018 | GPLGLQAHQV | 58 | 0.0021863621 | 0.00626477 |

TABLE 3
Identified CST (cancer specific transcript) antigens and derived
peptide antigen sequences indicating predicted HLA binding.

| | Cancer | | |
|---|---|---|---|
| | Specific | | |
| | Transcript | Predicted HLA Association | Encoded Cancer |
| SEQ ID | Antigen | Based on Patient Tissue | Specific Peptide |
| SEQ ID | EVA001 | C*07:02, A*02, C06:02, | SEQ ID NO: 18 |
| NO: 1 | | C07:01 | |
| SEQ ID | EVA002 | A*02:01 | SEQ ID NO: 19 |
| NO: 2 | | | |
| SEQ ID | EVA003 | B*07:02, C*04:01, B*07, | SEQ ID NO: 20, |
| NO: 3 | | B*35:03, B39:01 | SEQ ID NO: 21 |
| SEQ ID | EVA004 | B*07:02; C*07:02; B*07 | SEQ ID NO: 22 |
| NO: 4 | | C*06:02, C*12:03, B*51:01, | SEQ ID NO: 23 |
| | | C*05:01, C*06:02, C*03:03, | |
| | | C*01:02, C*08:01 | |
| SEQ ID | EVA005 | B*07:02, C*04:01 | SEQ ID NO: 24 |
| NO: 5 | | | |
| SEQ ID | EVA006 | C*06:02, B*08:01 | SEQ ID NO: 25 |
| NO: 6 | | | |
| SEQ ID | EVA007 | A*02:01 | SEQ ID NO: 26 |
| NO: 7 | | | |
| SEQ ID | EVA008 | C*07:02, A*03:01 | SEQ ID NO: 27, |
| NO: 8 | | | 28 and 56 |
| SEQ ID | EVA009 | A*02:01, C08:01 | SEQ ID NO: 29 |
| NO: 9 | | | SEQ ID NO: 30 |
| SEQ ID | EVA010 | B*07:02 | SEQ ID NO: 31 |
| NO: 10 | | | |
| SEQ ID | EVA011 | B*07:02 | SEQ ID NO: 32 |
| NO: 11 | | | |
| SEQ ID | EVA012 | B*39:01; C*04:01, | SEQ ID NO: 33 |
| NO: 12 | | B*39:01, A*02:01 | SEQ ID NO: 34 |
| SEQ ID | EVA013 | B*37:01, C*04:01 | SEQ ID NO: 35 |
| NO: 13 | | | |
| SEQ ID | EVA014 | B*08:01 | SEQ ID NO: 36 |
| NO: 14 | | | |
| SEQ ID | EVA015 | A*03:01, A*11:01, | SEQ ID NOs: |
| NO: 15 | | B*08:01, A*30:01, | 37-41 and 55 |
| | | A*31:01 | |
| SEQ ID | EVA016 | B*51:01; C*02:02; C*06:02, | SEQ ID |
| NO: 16 | | B*35:02; C*07:02, B*15:01; | Nos: 42-53 |
| | | C*03:03 | |
| SEQ ID | EVA017 | B*15:01; B*44:02; | SEQ ID NO: 54 |
| NO: 17 | | C*03:04; C*05:01 | |
| SEQ ID | EVA018 | B*07:02, A*11.01 | SEQ ID NO: 58 |
| NO: 57 | | | |

### Example 3—Transcript Expression by RT-qPCR in Tumour Vs Normal Tissue

Transcriptional expression of the antigen-encoding transcripts EVA001 to EVA018 (i.e. SEQ ID NO.1-17 and 57) was assessed across a panel of Esophageal Adenocarcinoma (EAC) tissue samples and cell lines by RT-qPCR.

Approximately 1000000 cells from tumour samples (human Esophageal Adenocarcinoma (EAC) tissue origin), was obtained for RNA extraction. RNA extraction was performed using Maxwell® RSC simplyRNA Cells Kit (Promega) according to manufacturer's instructions. RNA extractions were treated with DNAse enzymes to remove any contaminating genomic DNA. RNA was quantified and cDNA was synthesized from RNA samples with an RNA integrity number (RIN) above 6 and sufficient yield using iScript™ CDNA Synthesis Kit (Bio-Rad) according to manufacturer's instructions.

Real-time quantitative PCR (RT-qPCR) assays were performed for each candidate sequence using dye-labelled sequence-specific oligonucleotide probe (TaqMan hydrolysis probes) chemistry or SYBR Green dsDNA-binding dye chemistry. Amplification was carried out over a 75-200 bp region of each DA (DA=Dark Antigen, or CST antigen) candidate to avoid regions with long repeats of single bases and choose regions that have a GC content ranging between 50-60%.

Oligonucleotide primers in forward and reverse direction were synthesised for use in candidate DA (dark antigen) amplification and to cover the ORF coding region and the peptide sequences determined for each of SEQ ID NO.18 to SEQ ID NO.56 and 58, the assay nucleotide length was in each case around 130 bp (sequences not shown). The use of probes permits the detection of relevant transcript ORF of EVA001 to EVA018 using TaqMan chemistry.

The RT-qPCR assay procedure was carried out in two stages: (1) running a thermal gradient to optimise primer annealing temperature (TA) using a positive and negative cDNA sample for the DA expression designed based on the Cancer Cell Line Encyclopaedia (CCLE) RNASeq dataset; (2) running a standard curve using a serial dilution of plasmid DNA containing DA sequence with known copy number. PCR products of the thermal gradient RT-qPCR runs from (1) were kept at −20° C. until the best performing RT-qPCR assay was selected from (2), this is, the most sensitive, specific, and efficient assay. Upon selection, PCR products were purified, cloned into a backbone, and SANGER sequenced to confirm amplicon sequence.

The expression of the candidate DA was calculated relative to reference gene expression levels for genes PGK1 (Phosphoglycerate kinase 1) and TBP (TATA binding protein) which are expressed at a consistent level across EAC tissues and were therefore used as reference gene controls for the analysis. The expression of each EAC DA was calculated using the ACT method using the two reference genes (TBP and PGK1) as described below.

First, the average CT (Cq, or quantification cycle) of the reference genes is calculated:

$$CT ( ref ) = \frac{C T ( T B P ) + C T ( P G K 1 )}{2}$$

Second, the ΔCT method using the average of the reference genes is calculated:

$$Δ C T = {2}^{( C T ( r e f ) - C T ( D A ) )}$$

The results obtained reflect the relative expression of the DA to the reference genes.

### Example 4—Transcript Expression by RNAscope™ in Tumour Vs Normal Tissue

Transcript expression of DA EVA001-EVA018 (SEQ ID NO.1-17 and 57) was further validated experimentally across tissue microarrays (TMA) of esophageal adenocarcinoma (EAC), normal and multi-tumour tissues using RNA ISH (RNAscope™) using the BOND RX (Leica Biosystems) autostaining instrument. RNAscope™ enables detection of short RNA target sequences (mRNA or ncRNA)>300 nt. The methodology uses a probe designed for each CST sequence to hybridise to the region of the encoding candidate Dark Antigen transcript coding for the series of EVA001-EVA018, (SEQ ID NO.1-17 and 57), the probe in each case is designed to hybridise to a region including the sequence encoding the peptide sequence (i.e. SEQ ID NO: 18-56 and 58 in each respective case) and TMA quality control was performed to confirm the overall quality and integrity of RNA. A 20ZZ RNAscope probe was designed for each relevant EVA sequence, for example EVA001 transcript expression was detected by RNAscope probe EVA001-20zz-RNAscope which aligns to the region 1342-2769 of EVA001. EVA007 transcript expression was detected by RNAscope probe EVA007-20zz-RNAscope which aligns to the region 1623-2998 of EVA007. EVA006 transcript expression was detected by RNAscope probe EVA006-20zz-RNAscope which aligns to the region 774-1760 of EVA006. Probes for other EVA sequences were designed and manufactured accordingly. Dark Antigen (DA) staining of the tissues was then carried out and the expression of the esophageal adenocarcinoma (EAC) and esophageal squamous cell carcinoma (ESSC) candidate DA content is evaluated and reported for the tissues that were determined to have passed QC.

For the purposes of the quality control step and to evaluate RNA quality of the TMAs, treated slides of each TMA were stained with a negative (Bacillus subtilis dihydrodipicolinate reductase, DapB) and positive (Peptidylprolyl Isomerase B, PPIB) control probe. Some of the TMAs were additionally stained with a second positive control probe (ubiquitin C, UBC) to allow further QC of the RNA quality.

Once TMAs were stained, overall histological tissue integrity and sample RNA quality was evaluated. Tissue integrity was assessed by checking for presence of core, sufficient tissue regions of interest and adequate nuclear morphology based on a haematoxylin counterstain while RNA quality was evaluated following semi-quantitative scoring of tissue cores. Semi-quantitative scoring was used for QC and DA expression evaluation, and details of the score criteria for RNAscope can be found in Table 4 below, representative scoring of tissue is presented in FIG. 19.

For all TMAs, the core passed QC if PPIB score≥2 (relatively uniform throughout the tissue core) and DapB score<1 (No non-specific signal from DapB).

TABLE 4
RNA ISH semi-quantitative scoring criteria

| | Criteria |
|---|---|
| Score | RNAscope |
| 0 | No staining or <1 dot per 10 cells |
| 1 | 1 dot/cell |
| 2 | 2-3 dots/cell |
| 3 | 4-10 dots per cell and/or <10% dots |
| | are clusters |
| 4 | >10 dots per cell and/or <10% dots |
| | are clusters |

For the purposes of normal and tumour tissue screening the RNAscope probe was hybridised against each TMA in the study and whole slide scan images (at ×40 magnification) were used for evaluation.

As an example, FIG. 20 demonstrates the testing and detection reliability of the RNAscope tissue probe against tissue cores known by prior sequencing to express or not express either EVA001 transcript sequence (based on the CCLE dataset).

### Results and Discussion of Transcript Expression by RT-qPCR and RNAScope in Example 3 and Example 4

EVA001 (SEQ ID NO.1):

EVA001 (SEQ ID NO.1) demonstrates expression by RT-qPCR in several oesophageal adenocarcinoma (ESOAD) tissue samples (11/14) and tumour cell lines (8/8). Low to zero expression was observed in all oesophageal normal tissues adjacent to tumour (NAT). Very low to zero expression of EVA01 was observed across different normal tissues (FIG. 18). In most cases, when present, EVA001 is expressed at high levels relative to PGK1 and TBP reference genes. Assays included a positive control cell line Lung Carcinoma (NCI-H1299, indicated by “+”) which is known to express the EVA001 transcript and also included a negative control cell line Mammary Gland Adenocarcinoma (MCF7, indicated by “−”), known not to express EVA001 transcript

EVA001 transcript expression was assessed in normal tissues (FIG. 22) using RNAScope probe staining specific to the ORF region (probe EVA001-20zz). No positive signal (score≥1) was observed in any high-risk tissue (brain, heart, kidney, liver, lung), or any other normal tissues, apart from testis

RNAScope probe staining identified positive signal in 60 different tissues, across 9 different cancer indications for EVA001 (1/9 COAD cases, 4/9 H&N cases, 3/12 melanoma cases, 2/9 NSCLC cases, 1/10 ovarian ADC cases, 1/1 osteosarcoma and 2/9 bladder urothelial carcinoma cases showed a positive expression of EVA001, (FIG. 23). Of the 16 EAC tissues 7 were measured at level 1 or above highlighting that EVA001 has high prevalence in EAC tissues, i.e. oesophageal cancer (oesophageal adenocarcinoma) (FIG. 21). Additionally, EVA001 also showed moderate to high expression in tumour tissues of head and neck cancer (head and neck squamous cell carcinoma), melanoma, ovarian cancer (ovarian adenocarcinoma), lung cancer (non small cell lung cancer, NSCLC), bladder cancer (bladder ductal carcinoma), osteosarcoma.

EVA015 (SEQ ID NO.15):

EVA015 (SEQ ID NO.15) demonstrates expression by RT-qPCR in all oesophageal adenocarcinoma (ESOAD) tissue samples (14/14) and tumour cell lines (8/8). Low expression was observed in all oesophageal normal tissues adjacent to tumour (NAT). Low to zero expression of EVA015 was observed across different normal tissues (FIG. 32). With respect to cell lines low expression (1,000-5,000 copy number/10 ng of cDNA), moderate expression (5,000-10,000 copy number/10 ng of cDNA), high expression (>10,000 copy number/10 ng of cDNA). Positive control is indicated by “+” in the Figure.

EVA015 transcript expression was assessed in normal tissues (FIG. 33) using RNAScope probe staining specific to the ORF region (probe EVA015-20zz). No positive signal (score≥1) was observed in any high-risk tissue (brain, heart, kidney, liver, lung). Some positive signal (score≥1) was observed in 4/5 normal oesophagus, also in 3/3 testis, 3/3 lymph nodes, 3/3 thymus, 3/3 small intestines, 2/2 larynx, 1/3 bladders, 1/3 colons, 1/3 stomachs, 1/3 uterus. RNAScope probe staining identified positive signal in 60 different tissues, across 9 different cancer indications for EVA015 (12/12 COAD cases, 10/11 H&N cases, 10/11 melanoma cases, 8/10 NSCLC cases, 6/8 ovarian ADC cases, 6/8 pancreatic ADC cases, 4/8 breast carcinoma cases and 1/9 RCCC cases showed a positive expression of EVA015, FIG. 34. In addition to oesophageal cancer (oesophageal adenocarcinoma) where 100% of tissues showed moderate to high expression, EVA015 also showed moderate to high expression in tumour tissues of head and neck cancer (head and neck squamous cell carcinoma), melanoma, ovarian cancer (ovarian adenocarcinoma), lung cancer (non small cell lung cancer, NSCLC), colorectal cancer (colorectal adenocarcinoma COAD).

EVA004, Antigen 4, SEQ ID NO.4:

EVA004 (SEQ ID NO.4) demonstrates expression by RT-qPCR in all oesophageal adenocarcinoma tissue samples (10/10) and tumour cell lines (8/8) at a high to moderate level. Moderate expression was observed in all oesophageal normal tissues adjacent to tumour (NAT). Low to zero expression of EVA004 was observed across different normal tissues (FIG. 35), for example in Spleen (1/1), in Lung (2/2) and Brain (2/2). Positive control is indicated by “+” in the Figure.

EVA004 transcript expression was assessed in normal tissues (FIG. 36) using RNAScope probe staining specific to the ORF region (probe EVA004-20zz). No positive signal (score≥1) was observed in any high-risk tissue (brain, heart, kidney, liver, lung). Low positive signal (score≥1) was observed in 2/3 normal testis, 1/3 uterus.

RNAScope probe staining identified low level expression in oesophageal cancer (oesophageal adenocarcinoma) in 31.25% of tissues, EVA004 also showed low expression in tumour tissues of head and neck cancer (head and neck squamous cell carcinoma), melanoma, pancreatic cancer (pancreatic adenocarcinoma), lung cancer (non small cell lung cancer, NSCLC), colorectal cancer (colorectal adenocarcinoma COAD), FIG. 37.

EVA007 (SEQ ID NO.7) and EVA008 (SEQ ID NO.8):

EVA007 (SEQ ID NO.7) and EVA008 (SEQ ID NO.8) demonstrate expression by RT-qPCR in all oesophageal adenocarcinoma tissue samples (14/14) and tumour cell lines (8/8) at a high to moderate level. Four oesophageal NAT tissues showed moderate expression, normal tissues showed very low expression (FIG. 38/39), with spleen (1/1) moderate in both cases. Positive control is indicated by “+” in the Figure.

EVA007/EVA008 are transcribed from the same RNA transcript hence can be detected using the same probe in RNAScope analysis. EVA007/EVA008 transcript expression was assessed in normal tissues (FIG. 40) using RNAScope probe staining specific to the ORF region (probe EVA007008-20zz). No positive signal (score≥1) was observed in any high-risk tissue (brain, heart, kidney, liver, lung). Expression was seen in oesophagus (4/5), thymus (3/3), testis (3/3), and to a lesser extent in small intestine (3/3), lymph nodes (2/2), larynx (2/2), and low levels in in fallopian tubes, ovary (2/3), tonsil (1/3), cervix (1/2).

RNAScope probe staining, (FIG. 41), identified moderate to high level expression in oesophageal cancer (oesophageal adenocarcinoma) in 100% of tissues, EVA007 also showed low expression in tumour tissues of head and neck cancer (head and neck squamous cell carcinoma), melanoma, pancreatic cancer (pancreatic adenocarcinoma), lung cancer (non small cell lung cancer, NSCLC), colorectal cancer (colorectal adenocarcinoma COAD), breast cancer (breast ductal carcinoma) and bladder cancer (bladder ductal carcinoma).

EVA005 (SEQ ID NO.5):

EVA005 (SEQ ID NO.5) demonstrates expression by RT-qPCR in all oesophageal adenocarcinoma tissue samples (13/14) and tumour cell lines (8/8) at a high to moderate level. One oesophageal NAT tissue showed low expression, normal tissues showed very close to zero expression (FIG. 42). Positive control is indicated by “+” in the Figure.

EVA005 transcript expression was assessed in normal tissues (FIG. 43) using RNAScope probe staining specific to the ORF region (probe EVA005-20zz). No positive signal (score≥1) was observed in any high-risk tissue (brain, heart, kidney, liver, lung). Expression was seen at low level in oesophagus (2/5), colon (2/5), thymus (2/3), lymph nodes (1/2), larynx (1/2), and uterus (1/3) and high levels in, testis (3/3).

RNAScope probe staining, (FIG. 44), identified moderate to high level expression in oesophageal cancer (oesophageal adenocarcinoma) in 50% of tissues, EVA005 also showed moderate expression in tumour tissues of head and neck cancer (head and neck squamous cell carcinoma), melanoma, lung cancer (non small cell lung cancer, NSCLC), and to a lesser extent in colorectal cancer (colorectal adenocarcinoma COAD), pancreatic cancer (pancreatic adenocarcinoma), breast cancer (breast ductal carcinoma), bladder cancer (bladder ductal carcinoma), renal cancer (renal clear cell carcinoma) and ovarian cancer (ovarian adenocarcinoma).

EVA006 (SEQ ID NO.6):

EVA006 (SEQ ID NO.6) demonstrates expression by RT-qPCR in some oesophageal adenocarcinoma tissue samples (6/14) most strongly in late stage (III and IV) tumour tissues. There was no expression observed in oesophageal NAT tissue or normal tissues. No expression was seen in oesophageal tumour cell lines. (FIG. 45). Positive control is indicated by “+” in the Figure.

EVA006 transcript expression was assessed in normal tissues (FIG. 46) using RNAScope probe staining specific to the ORF region (probe EVA006-20zz). No positive signal (score≥1) was observed in any high-risk tissue (brain, heart, kidney, liver, lung) or any other normal tissue.

RNAScope probe staining, (FIG. 47), identified high level expression in oesophageal cancer (oesophageal adenocarcinoma) in 100% of tissues, EVA006 also showed high expression in tumour tissues of breast cancer (breast ductal carcinoma), bladder cancer (bladder ductal carcinoma), colorectal cancer (colorectal adenocarcinoma COAD), lung cancer (non small cell lung cancer, NSCLC) and pancreatic cancer (pancreatic adenocarcinoma), and moderate expression in head and neck cancer (head and neck squamous cell carcinoma) and ovarian cancer (ovarian adenocarcinoma).

It was noted that RNAScope probe staining signal of tumour tissue sections is only detected in the stromal regions of the tumour, which could also indicate a correlation of expression with a more advanced/aggressive disease state most notably for the more stromal cancers such as breast cancer/carcinoma and pancreatic cancer/adenocarcinoma, (as well as GIST cancers including colorectal and oesophageal) particularly where the cancer is more advanced stage as it was further noted that the majority of expression was seen in tumour tissue samples with cancer spread to nodes.

EVA013 (SEQ ID NO.13):

EVA013 (SEQ ID NO.13) demonstrates expression by RT-qPCR in all oesophageal adenocarcinoma tissue samples (13/13) and tumour cell lines (8/8) at a high to moderate level. All oesophageal NAT tissue showed some expression of EVA013 some at a moderate to high level. Normal tissues showed generally low expression with some moderate expression in spleen (FIG. 48). Positive control is indicated by “+” in the Figure.

EVA013 transcript expression was assessed in normal tissues (FIG. 49) using RNAScope probe staining specific to the ORF region (probe EVA014-20zz). No positive signal (score≥1) was observed in any high-risk tissue (brain, heart, kidney, liver, lung). Low expression was seen in oesophagus (1/5), colon (1/3), thymus (1/3), lymph nodes (1/2), moderate levels in larynx (1/1), and uterus (1/4) and moderate to high levels in, testis (5/5).

RNAScope probe staining, (FIG. 50), identified low level expression in oesophageal cancer (oesophageal adenocarcinoma) in 45% of tissues. Positive signal (score≥1) was observed in 11 different donors, across 5 different cancer indications. EVA014 showed low expression in tumour tissues of head and neck cancer (head and neck squamous cell carcinoma), melanoma, lung cancer (non small cell lung cancer, NSCLC); [to a lesser extent in breast cancer (breast ductal carcinoma), bladder cancer (bladder ductal carcinoma)].

EVA011 (SEQ ID NO.11):

EVA011 (SEQ ID NO.11) demonstrates expression by RT-qPCR in all oesophageal adenocarcinoma tissue samples (13/13) and tumour cell lines (8/8) at a high to moderate level. All oesophageal NAT tissue showed some expression of EVA011 some at a moderate to high level. Normal tissues showed generally low expression with some moderate expression in spleen (FIG. 51). Positive control is indicated by “+” in the Figure.

EVA011 transcript expression was assessed in normal tissues (FIG. 52) using RNAScope probe staining specific to the ORF region (probe EVA014-20zz). No positive signal (score≥1) was observed in any high-risk tissue (brain, heart, kidney, liver, lung). Low to moderate expression was seen in oesophagus (5/5), and low expression in colon (1/3), cervix (1/3), small intestine (2/3), larynx (1/2), skin (1/3) and stomach (1/3).

RNAScope probe staining, (FIG. 53), identified moderate to low level expression in oesophageal cancer (oesophageal adenocarcinoma) in 50% of tissues. Positive signal (score≥1) was observed in 17 different donors, across 10 different cancer indications. EVA011 showed high expression in pancreatic cancer (pancreatic adenocarcinoma) 5/6 tissues, low to moderate expression in tumour tissues of head and neck cancer (head and neck squamous cell carcinoma) 5/11 tissues, low expression in lung cancer (non small cell lung cancer, NSCLC) 3/10; and colorectal cancer (colorectal adenocarcinoma) 3/10 and [to a lesser extent in bladder cancer (bladder ductal carcinoma)].

### Observations on EVA001, EVA006 and EVA007:

Transcript expression for each of EVA001, EVA006 and EVA007 was evaluated by RNAscope™ across major healthy tissues, GI (gastrointestinal) tumour tissues (esophageal adenocarcinoma, EAC tissue samples) and across a number of generalised tumour tissue types including, melanoma, non small cell lung cancer, ovarian cancer, pancreatic cancer and breast cancer. Data presented in FIG. 27 confirms that there is no transcript expression for any of EVA001, EVA006 or EVA007 across major healthy tissues. Transcript staining in FIG. 28 confirms that both EVA001 (score between 3 and 4) and EVA007 (score between 1 and 2) are highly expressed in GI tumor tissue, transcript staining for EVA006 (score between 1 and 2) was notable as much more of the staining was observed in the stromal cells relative to the tumor cells indicating that the transcript targeting would be much more strongly directed towards the tumor stromal compartments. FIG. 29 confirms that each of the transcripts were strongly detected at high level across multiple other major solid tumor indications, EVA001 in melanoma and ovarian tumor tissue, EVA007 in breast and non small cell lung cancer tissue and particularly EVA006 which is robustly expressed at high prevalence in stromal rich cancers or tumors such as breast cancers (69% of tumor cells) and pancreatic cancer/adenocarcinoma (81% of tumor cells).

### Conclusions:

The transcript expression data determined by RT-qPCR and RNAScope show EVA001, EVA004, EVA005, EVA006, EVA007, EVA008, EVA011, EVA013, EVA015, Dark Antigen transcripts expression at a high level in EAC tissues as shown by RNA in situ hybridisation (RNA ISH). Negligible normal tissue expression was detected (no positive signal (score≥1) was observed in any high-risk tissue, e.g. brain, heart, kidney, liver, lung and no positive signal (score≥1) was observed in normal c), indicating that these antigens are cancer specific and validated therapeutic targets for their indicated cancer therapy, (see also Table 1 herein), for example in ovarian cancer, gastrointestinal cancer, pancreatic cancer, prostate cancer, stromal cancer, colon cancer, breast cancer, skin cancer, melanoma, head and neck cancer, sarcoma, rectal cancer, lung cancer (e.g. LUSC, LUAD, NSCLC), stomach cancer, cervical cancer, uterine cancer, osteosarcoma, oesophageal cancer, oesophageal adenocarcinoma (EAC) and oesophageal squamous cell carcinoma (ESSC), particularly oesophageal cancer, or EAC or ESSC. It is noted that EVA006 is strongly indicated as a therapeutic targets for stromal rich cancer therapy, for example breast, pancreatic, prostate and gastric cancers.

### Example 5—Immunogenicity of Peptide Derived from CST Antigens: The CST

antigens identified by the preceding Immunopeptidomics analysis and subsequently detected in esophageal adenocarcinoma (EAC) and esophageal squamous cell carcinoma (ESSC) were further tested for immunogenicity against donor PBMCs. The aim is to investigate the ability to identify the existence of potentially therapeutically relevant immune responses to the CST antigens in subjects or cancer patients.

A cytokine release assay can show that CST antigen-specific CD8 T-cells are present in the normal T-cell repertoire of healthy individuals. Due to the expression of CST antigens in naïve and thymic tissues, the T cells have not been deleted by central tolerance. This type of assay comprises multiple steps carried out over 7 weeks. Step 1: PBMC, CD14 Monocytes, CD19⁺ B cells and Naïve CD8 T-cells are isolated from the peripheral blood of normal blood donors (which is HLA typed); these cells are HLA Class I-typed to match the specific binding predictions of CST antigens being tested. In the present case the assays were carried out in multiple HLA donors matched to the relevant CST sequence (Table 3). The CD14 are cultured and matured into dendritic cells through specific cytokine cocktails over 9 days, B cells are expanded on an hCD40L-transduced NIH/3T3 cell line over 2 weeks. Step 2: Mature dendritic cells are pulsed with individual CST antigen peptide for 4 hours prior to being co-cultured with isolated Naïve CD8 T-cells for 9 days. Step 3: The CD8 T-cells undergo 3 restimulations 7 days apart with peptide pulsed expanded B cells. Step 4: The readout ELISpot assay is performed to detect IFNgamma released by activated CD8 T-cells. CD8 T-cells are cultured and activated with peptides in the Elispot plate coated with IFNgamma capture antibodies. Following overnight activation, the cells are washed from the plate and IFN gamma captured on the plate is detected with detection antibodies and a conjugated streptavidin-substrate reaction that forms a purple precipitation where IFNgamma was captured.

Data derived from such assays includes spot count, median spot size and median spot intensity. These are indicators of frequency of T-cells producing IFN gamma and amount of IFN gamma per cell. Comparisons of the responses to CST antigens and control antigens demonstrate that naïve subjects contain a robust repertoire of CST antigen-reactive T-cells that can be expanded by vaccination with CST antigen-based immunogenic formulations.

### Results of Immunogenicity Assays:

FIGS. 24 and 25 show significant CD8 T-cell responses from normal blood donors to the HLA restricted peptides from the CST antigen peptides (EVA001 derived peptide, SEQ ID NO:18 and EVA007 derived peptide SEQ ID NO:26). The data in FIGS. 30 and 31 indicates that for EVA006 derived peptide SEQ ID NO:25, there was a low but observable incidence of responsive T-cell immunogenicity in the T-cells derived from the single normal donor sample tested.

FIGS. 54 and 55 show CD8 T-cell responses from normal blood donors to the HLA restricted peptides from the CST antigen peptides: EVA015 derived peptides, SEQ ID NOs:37, 38, 39, EVA006 derived peptide SEQ ID NO:25, EVA005 derived peptide SEQ ID NO:24, EVA008 derived peptide SEQ ID NO:28.

FIG. 56 shows CD8 T-cell responses from normal blood donors to the HLA restricted peptides from the CST antigen peptides: EVA006 derived peptide SEQ ID NO: 25, EVA004 derived peptide SEQ ID NO:22.

FIG. 57 shows CD8 T-cell responses from normal blood donors to the HLA restricted peptides from the CST antigen peptides: EVA005 derived peptide SEQ ID NO: 24, EVA008 derived peptide SEQ ID NO:27 and EVA015 derived peptide, SEQ ID NOs:37,

FIG. 58 shows CD8 T-cell responses from normal blood donors to the HLA restricted peptides from the CST antigen peptides: EVA002 derived peptide SEQ ID NO: 19, EVA005 derived peptide SEQ ID NO:24 and EVA007 derived peptide, SEQ ID NO: 26, and EVA008 derived peptide, SEQ ID NOs:27.

In conclusion, Evidence for immunogenicity was observed for the following antigenic peptides: EVA001 peptide, SEQ ID NO:18; EVA002 peptide, SEQ ID NO:19; EVA004 derived peptide SEQ ID NO:22; EVA005 derived peptide SEQ ID NO:24; EVA006 derived peptide SEQ ID NO:25; EVA007 peptide SEQ ID NO:26; EVA008 derived peptides SEQ ID NO:27 and SEQ ID NO:28; EVA015 derived peptides, SEQ ID NOs:37, 38, 39. Compelling evidence for immunogenicity was observed in 1/25 individual C*07:02 donors for KRYNRIMHDEL, SEQ ID NO:18, peptide sequence derived from CST Antigen 1 from SEQ ID NO.1 EVA001 and in 1/16 individual A*02:01 donors for AVSLTILAV, SEQ ID NO: 26, peptide sequence derived from CST Antigen 7 SEQ ID NO.7 EVA007.

### Example 6: Antigen Specific TCR Isolation

PBMC and CD8 T-cells were isolated from the peripheral blood of HLA-matched healthy blood donors. The isolated CD8 population was then stained with a DNA barcoded MHC Dextramer reagent and flow sorted to identify peripheral CD8 T-cells recognising the pHLA (specific target peptide-HLA complex) of interest. Sorted cells were then taken for single cell sequencing and immune profiling using the 10x Chromium platform (10× Genomics). TCR sequences were subsequently identified with the use of Cell Ranger software (10× Genomics) which performs sample demultiplexing, barcode processing, single cell 3′ and 5′ gene counting, V (D) J transcript sequence assembly and annotation, and barcode analysis from single cell data. Data was further subjected to in-house computational analysis. This analysis was performed for the antigen peptides derived from EVA015 (SEQ ID NO:15), in particular peptide antigen SEQ ID NO:37 SLIKQPPRK using peripheral blood of 12 separate HLA-matched healthy blood donors, total of 17 T-cell clones were identified from each of which antigen specific TCRs were isolated, cloned and sequenced (Table 5).

Using the same procedure T-cell clones were identified and antigen specific TCRs were isolated, cloned and sequenced for (i) the antigen peptides derived from EVA001 (SEQ ID NO:1), in particular peptide antigen SEQ ID NO:18 KRYNRIMHDEL and (ii) for the antigen peptides derived from EVA007 (SEQ ID NO: 7), in particular peptide antigen SEQ ID NO:26, AVSLTILAV (see Table 5).

TABLE 5
Antigen-specific, single cell sorting and TCR isolation
for peptide antigen SEQ ID NO: 37 SLIKQPPRK from specifically
immunoreactive T-cells derived from peripheral blood
of HLA-matched healthy blood donors.

| | | | No. of antigen |
|---|---|---|---|
| | | | specific TCR |
| | | Number of | sequences |
| Donor | | T-cells | isolated |
| Identifier | EVA Sequence | sorted | from 10x |
| 1 | SEQ ID NO: 37 | 14184 | 2 |
| 2 | SEQ ID NO: 37 | 26533 | 4 |
| 3 | SEQ ID NO: 37 | 2927 | 0 |
| 3 | SEQ ID NO: 37 | 2917 | 2 |
| 4 | SEQ ID NO: 37 | 19460 | 1 |
| 5 | SEQ ID NO: 37 | 50320 | 10 |
| 6 | SEQ ID NO: 37 | 1865 | 0 |
| 5 | SEQ ID NO: 37 | 5425 | 0 |
| 7 | SEQ ID NO: 37 | 2011 | 0 |
| 8 | SEQ ID NO: 37 | 4134 | 0 |
| 9 | SEQ ID NO: 37 | 10762 | 5 |
| 10 | SEQ ID NO: 37 | 5167 | 0 |
| 11 | SEQ ID NO: 37 | 9593 | 3 |
| 12 | SEQ ID NO: 37 | 6380 | 0 |
| 13 | SEQ ID NO: 18 | 681 | 10 |
| 14 | SEQ ID NO: 18 | 135 | 4 |
| 15 | SEQ ID NO: 26 | 336 | 10 |

### In Conclusion:

Specifically immunoreactive T-cells for antigenic peptides derived from EVA001, EVA007 and EVA015, in particular antigenic peptides SEQ ID NO:18, SEQ ID NO:26 and SEQ ID NO:37 were isolated. In each of the T-cell clones the specifically immunoreactive TCRs recognizing each antigenic peptide were obtained, cloned and isolated and further subjected to sequencing of TCR chains.

1. An isolated polypeptide comprising the amino acid sequence of SEQ ID NO: 7 or 26.

2. An isolated nucleic acid encoding the polypeptide according to claim 1.

3. A vector comprising the nucleic acid according to claim 2.

4. The vector according to claim 3, wherein the vector is a viral vector.

5. A pharmaceutical composition comprising the polypeptide according to claim 1 and a pharmaceutically acceptable carrier.

6. The pharmaceutical composition according to claim 5, further comprising one or more immunostimulants.

7. The pharmaceutical composition according to claim 6, wherein the one or more immunostimulants are selected from aluminum salts, saponins, immunostimulatory oligonucleotides, oil-in-water emulsions, aminoalkyl glucosaminide 4-phosphates, lipopolysaccharides and derivatives thereof and other TLR4 ligands, TLR7 ligands, TLR8 ligands and TLR9 ligands, IL12, interferons, Incomplete Freund's adjuvant (IFA), aluminum-based adjuvants, TLR agonists, synthetic double-stranded RNAs (dsRNAs), glucopyranosyl lipid a (GLA), imidazoquinolines, CPG oligodeoxynucleotides (ODNs), cyclic dinucleotides (CDNs), manganese-based adjuvants; metabolic adjuvants, nanoparticle-based adjuvant, water-in-oil nanoemulsions, micro or nanoparticle adjuvants, CGAS-STING/STING agonists, cytokines, self-assembling peptides, virus-like particles (VLPs), inorganic nanoparticles, caged protein nanoparticles, nucleoside-unmodified mRNA, PRR ligands (pattern recognition receptor ligands), mRNA encoding functional proteins, component lipid-based adjuvants, peptide and glycolipid immunostimulants, and TLR agonists.

8. A method of eliciting an immune response in a human subject, the method comprising administering to the human subject the pharmaceutical composition according to claim 5.

9. The method according to claim 8, wherein the immune response is elicited against a tumor expressing a polypeptide comprising the amino acid sequence of SEQ ID NO: 26.

10. An isolated antigen-binding polypeptide which is immunospecific for the polypeptide according to claim 1.

11. The antigen-binding polypeptide according to claim 10, wherein the antigen-binding polypeptide is a monoclonal antibody or a fragment thereof or T cell receptor or fragment thereof.

12. The antigen-binding polypeptide according to claim 10, wherein the antigen-binding polypeptide is coupled to a cytotoxic moiety.

13. A pharmaceutical composition comprising the antigen-binding polypeptide according to any claim 11 and a pharmaceutically acceptable carrier.

14. A method of treating a cancer in a human subject in need thereof, wherein the cells of the cancer express the amino acid sequence of SEQ ID NO: 7, the method comprising: administering to the human subject the pharmaceutical composition of claim 13.

15. The method according to claim 14, wherein the cancer is selected from the group consisting of esophageal cancer, head and neck cancer, melanoma, lung cancer, colorectal cancer, pancreatic cancer, breast cancer, and bladder cancer.

16. A cell that has been engineered to express the antigen-binding polypeptide according to claim 10 on its surface.

17. The cell according to claim 16, wherein the cell is an immune cell.

18. A method of treating a cancer in a human subject, wherein the cells of the cancer express the amino acid sequence of SEQ ID NO: 7, the method comprising: administering to the human subject the cell of claim 16.

19. The method according to claim 18, wherein the cancer is selected from the group consisting of esophageal cancer, head and neck cancer, melanoma, lung cancer, colorectal cancer, pancreatic cancer, breast cancer, and bladder cancer.

20. A method of treating a cancer in a human subject, the method comprising administering to the human subject a pharmaceutical composition comprising an isolated polypeptide comprising the amino acid sequence of SEQ ID NO: 7 or 26.

21. The method according to claim 20, wherein the cancer is selected from the group consisting of esophageal cancer, head and neck cancer, melanoma, lung cancer, colorectal cancer, pancreatic cancer, breast cancer, and bladder cancer.

22. The method according to claim 21, wherein the cancer is esophageal cancer.


SEQUENCE LISTING (63 sequences)

SEQ ID NO: 1 - protein, 13 aa, Homo sapiens
MKRYNRIMHDELT

SEQ ID NO: 2 - protein, 36 aa, Homo sapiens
VVLPGTLCFMKLLKLNMGSLAPRSALTLETPAKYVR

SEQ ID NO: 3 - protein, 53 aa, Homo sapiens
LAANSVSVTSVPLWCLYSISLASSLMHPKTDAALLSPVPSQQTRGCFYPCLDF

SEQ ID NO: 4 - protein, 44 aa, Homo sapiens
LARETLLPASSAFPSSPFRTVPHLLLAQCCLKMQLLFANNRNTS

SEQ ID NO: 5 - protein, 53 aa, Homo sapiens
LSGMSLSGVMIKLYSPLFMTRYNSPSPLKSQLLVSLSWTSVIGRLLMSIFNLC

SEQ ID NO: 6 - protein, 23 aa, Homo sapiens
MLKTNNKKQTTKKKLKKKTEIEL

SEQ ID NO: 7 - protein, 18 aa, Homo sapiens
MLIAVSLTILAVLIFWIN

SEQ ID NO: 8 - protein, 40 aa, Homo sapiens
MQKVPDVLWNRKLRKKKRSFKRSSPVNSVLSKHWTRSASR

SEQ ID NO: 9 - protein, 19 aa, Homo sapiens
VEMPAIKWHPDGLIHITLL

SEQ ID NO: 10 - protein, 59 aa, Homo sapiens
MSQGRASPFTTSRPRPGKGSLWRRRFVREEGEAPTHLGLTRSFAKRLGGVPGATKLPPS

SEQ ID NO: 11 - protein, 22 aa, Homo sapiens
MDSPIPYHLSPTLAPEILNSTM

SEQ ID NO: 12 - protein, 57 aa, Homo sapiens
TFLELKLFSTEEDRADSRDHGVSLGPSPQMVHPLAEAPAHSLTSNLLEPAHHCQAHY

SEQ ID NO: 13 - protein, 34 aa, Homo sapiens
VQNSVDSSLSLLWRSSIPYLEMTLFMCLSLVMML

SEQ ID NO: 14 - protein, 12 aa, Homo sapiens
MASRKKMIGFVK

SEQ ID NO: 15 - protein, 56 aa, Homo sapiens
LFKMPQVKHQRRNLRDPLPNRSLIKQRPPRKWQSPTLASFQLGSRLLTTPPCPTRK

SEQ ID NO: 16 - protein, 247 aa, Homo sapiens
MPGRPPPTVGPLISPVAPRLPFTFGPSKLKSTFGAEIRGPFRFTSTPGPFSSPETCGPLALTCKSDPGVEMPNWGSFILGSLMLPSDASKLRSGAPTDTLGHLMSPETARSPCRLGPLSVTSGAPTLSFVVASIPGTFTPSPPTCILPPTPLRLSPGACTFMLGTDASRSPFKLGPFKFRSRSGPETFGIERFTEGKSTPGPFRETSGPSSLTSGPLKSPSREGSWAWTSAPPPSIFTPGMPILGME

SEQ ID NO: 17 - protein, 23 aa, Homo sapiens
VYNIKMKGFVLHCEVQAEVGIRF

SEQ ID NO: 18 - protein, 11 aa, Homo sapiens
KRYNRIMHDEL

SEQ ID NO: 19 - protein, 10 aa, Homo sapiens
KLLKLNMGSL

SEQ ID NO: 20 - protein, 9 aa, Homo sapiens
HPKTDAALL

SEQ ID NO: 21 - protein, 12 aa, Homo sapiens
SSLMHPKTDAAL

SEQ ID NO: 22 - protein, 9 aa, Homo sapiens
FPSSPFRTV

SEQ ID NO: 23 - protein, 9 aa, Homo sapiens
FRTVPHLLL

SEQ ID NO: 24 - protein, 9 aa, Homo sapiens
SPSPLKSQL

SEQ ID NO: 25 - protein, 9 aa, Homo sapiens
LKKKTEIEL

SEQ ID NO: 26 - protein, 9 aa, Homo sapiens
AVSLTILAV

SEQ ID NO: 27 - protein, 9 aa, Homo sapiens
KRSSPVNSV

SEQ ID NO: 28 - protein, 12 aa, Homo sapiens
KRSSPVNSVLSK

SEQ ID NO: 29 - protein, 8 aa, Homo sapiens
GLIHITLL

SEQ ID NO: 30 - protein, 10 aa, Homo sapiens
HPDGLIHITL

SEQ ID NO: 31 - protein, 9 aa, Homo sapiens
RPRPGKGSL

SEQ ID NO: 32 - protein, 9 aa, Homo sapiens
SPTLAPEIL

SEQ ID NO: 33 - protein, 8 aa, Homo sapiens
SRDHGVSL

SEQ ID NO: 34 - protein, 10 aa, Homo sapiens
VSLGPSPQMV

SEQ ID NO: 35 - protein, 8 aa, Homo sapiens
VDSSLSLL

SEQ ID NO: 36 - protein, 8 aa, Homo sapiens
MASRKKMI

SEQ ID NO: 37 - protein, 10 aa, Homo sapiens
SLIKQRPPRK

SEQ ID NO: 38 - protein, 10 aa, Homo sapiens
RSLIKQRPPR

SEQ ID NO: 39 - protein, 9 aa, Homo sapiens
SLIKQRPPR

SEQ ID NO: 40 - protein, 9 aa, Homo sapiens
DPLPNRSLI

SEQ ID NO: 41 - protein, 11 aa, Homo sapiens
RSLIKQRPPRK

SEQ ID NO: 42 - protein, 9 aa, Homo sapiens
ILPPTPLRL

SEQ ID NO: 43 - protein, 10 aa, Homo sapiens
GAPTLSFVVA

SEQ ID NO: 44 - protein, 9 aa, Homo sapiens
GSFILGSLM

SEQ ID NO: 45 - protein, 11 aa, Homo sapiens
LMLPSDASKLR

SEQ ID NO: 46 - protein, 10 aa, Homo sapiens
LRSGAPTDTL

SEQ ID NO: 47 - protein, 13 aa, Homo sapiens
RFTEGKSTPGPFR

SEQ ID NO: 48 - protein, 9 aa, Homo sapiens
ILPPTPLRL

SEQ ID NO: 49 - protein, 10 aa, Homo sapiens
KLKSTFGAEI

SEQ ID NO: 50 - protein, 9 aa, Homo sapiens
SFILGSLML

SEQ ID NO: 51 - protein, 10 aa, Homo sapiens
GAPTLSFVVA

SEQ ID NO: 52 - protein, 9 aa, Homo sapiens
GSFILGSLM

SEQ ID NO: 53 - protein, 10 aa, Homo sapiens
TSGPLKSPSR

SEQ ID NO: 54 - protein, 9 aa, Homo sapiens
VQAEVGIRF

SEQ ID NO: 55 - protein, 9 aa, Homo sapiens
DPLPNRSLI

SEQ ID NO: 56 - protein, 12 aa, Homo sapiens
KRSSPVNSVLSK

SEQ ID NO: 57 - protein, 40 aa, Homo sapiens [sequence omitted]

SEQ ID NO: 58 - protein, 10 aa, Homo sapiens
GPLGLQAHQV

SEQ ID NO: 59 - protein, 13 aa, synthetic construct
AKFVAAWTLKAAA

SEQ ID NO: 60 - protein, 13 aa, synthetic construct
PKYVKQNTLKLAT

SEQ ID NO: 61 - protein, 14 aa, synthetic construct
QYIKANSKFIGITE

SEQ ID NO: 62 - protein, 21 aa, synthetic construct
FNNFTVSFWLRVPKVSASHLE

SEQ ID NO: 63 - protein, 17 aa, synthetic construct
ISQAVHAAHAEINEAGR